(19) United States
(12) Patent Application Publication
(10) Pub. No.: US 2008/0196111 A1
(43) Pub. Date: Aug. 14, 2008

(54) Expression Vectors and Methods for Obtaining Nk Cell Specific Expression

(75) Inventors: Eric Vivier, Cassis (FR); Mathieu Blery, Marseille (FR); Thierry Walzer, Marseille (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Innate Pharma S.A., Marseille (FR)

(21) Appl. No.: 11/909,021

(22) PCT Filed: Mar. 17, 2006
(86) PCT No.: PCT/IB06/01208

§ 371 Date: Sep. 18, 2007

Related U.S. Application Data
(60) Provisional application No. 60/663,362, filed on Mar. 18, 2005.

(51) Int. Cl. A01K 67/027; C12N 15/87; C12N 5/06; C12Q 1/66

(52) U.S. Cl. 800/3; 435/455; 435/8; 800/13; 435/325

(57) ABSTRACT

The present invention relates to expression constructs and methods of specifically marking NK cells in nonhuman mammals. Specifically, methods are presented that allow the specific expression of foreign genes in the NK cells of a nonhuman mammal. Such methods are useful for the generation of animal models for disorders involving NK cells, and for the evaluation of genes or compounds with respect to their effects on NK cells.

### FIELD OF THE INVENTION

The present invention relates to expression constructs and methods of specifically marking NK cells in nonhuman mammals. Specifically, methods are presented that allow the specific expression of foreign genes in the NK cells of a nonhuman mammal. Such methods are useful for the generation of animal models for disorders involving NK cells, and for the evaluation of genes or compounds with respect to their effects on NK cells.

### BACKGROUND

Natural killer (NK) cells are a subpopulation of lymphocytes that are involved in non-conventional immunity. Characteristics and biological properties of human NK cells include the expression of surface antigens including CD56 and/or CD16, the absence of the alpha/beta or gamma/delta TCR complex on the cell surface; the ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

NK cells provide an efficient imrnmunosurveillance mechanism by which undesired cells such as tumor or virally-infected cells can be eliminated without prior sensitization. NK cells also produce cytokines, such as IFN-gamma, upon activation. NK cells are also thought to be important in initiating adaptive immune responses and in regulating autoimmune responses. Because of the importance of NK cells in warding off infection or cancer, and in preventing autoimmune disease, it is likely that alterations in the number or activity of NK cells can contribute to the onset or treatment of many diseases.

NK cell activity is regulated by a complex mechanism that involves both activating and inhibitory signals. Several distinct NK-specific receptors, called NCRs, have been identified that play an important role in the NK cell mediated recognition and killing of target cells, for example tumor cells or cells infected by microbes. These receptors, including NKp30, NKp46 and NKp44, are members of the Ig superfamily. Their cross-linking, induced by specific mAbs, leads to a strong NK cell activation resulting in increased intracellular Ca⁺⁺ levels, triggering of cytotoxicity, and lymphokine release. Importantly, mAb-mediated blocking of NKp30, NKp46, and/or NKp44 results in a blocking of NK cytotoxicity against certain target cells that would normally be lysed by the NK cells. These findings provide evidence for a central role of these receptors in natural cytotoxicity.

NK cells are negatively regulated by major histocompatibility complex (MHC) class I-specific inhibitory receptors (Kärre et al. (1986) Nature 319:675-8; Ohlen et al, (1989) Science 246:666-8). These specific receptors bind to polymorphic determinants of major histocompatibility complex (MHC) class I molecules or HLA and inhibit natural killer (NK) cell lysis. In humans, certain members of a family of receptors termed killer Ig-like receptors (KIRs) recognize groups of HLA class I alleles.

While some animal models exist in which NK cell activity or number is altered (see, e.g., JAX|NOTES No. 489, Spring 2003, from the Jackson Laboratory, and Chiesa S. et al, Mol Immunol. (2005) 42(4):477-484, the entire disclosures of both of which are herein incorporated by reference), such animal models generally involve mutations that either have a limited effect on NK cells, or affect cell types other than NK cells, thus limiting their utility for the specific study of NK cells under various conditions. Indeed, there is a great need in the art for new tools that would enable researchers to specifically manipulate NK cells in vivo, e.g., by specifically altering their activity, number, receptor profile, or other properties, or by allowing them to be marked in living animals, enabling their close and precise monitoring under various conditions. The present invention addresses these and other needs.

### SUMMARY OF THE INVENTION

The present invention provides methods and compositions for the expression of any nucleic acid of interest, for example including but not limited to any cDNA or genomic DNA sequence, and thus generally for driving the specific expression of any heterologous protein or RNA, in mammalian NK cells and precursor cells thereof (e.g. CD34+ precursor cells and/or stem cells). In certain embodiments, the methods are used to specifically mark NK cells of nonhuman mammals, e.g. for testing the impact of test compounds on NK cell activity or number. In other embodiments, RNAs or proteins with a potential ability to alter NK cell activity, number, or any other property can be specifically expressed in NK cells, e.g. to develop animal models for NK cell related disorders.

Accordingly, the present invention provides an expression construct comprising a mammalian NCR promoter operably linked to a heterologous sequence encoding a protein or functional RNA. In another aspect, the present invention provides NK cells comprising any of the herein-described expression constructs. In a preferred embodiment, the NCR is NKp46. In one example, the promoter comprises the 400 bp region immediately upstream of the start codon (ATG) of the NKp46 gene, in particular od SEQ ID No 54.

In one embodiment the heterologous coding sequence encodes a protein known to be expressed by NK cells, a protein specifically expressed by NK cells, or a protein known to be expressed by cells other than NK cells (and/or not known to be expressed by NK or NK-like cells in their natural environment). In one embodiment, the NCR is NKp46. In some embodiments, the NCR is from a first species (either human or non-human) and the heterologous coding sequence encodes a protein from a second species, which may also be either human or non-human, wherein said first and said second species are not the same. In preferred embodiments, both the NCR and the heterologous coding sequence are from the same species; preferably the NCR and heterologous coding sequence are of human origin.

In a preferred embodiment, the heterologous coding sequence encodes a protein known to be expressed by an NK cell. Preferably the heterologous coding sequence encodes an NK cell activating receptor or an NK cell inhibiting receptor. In a first embodiment, both the NCR and the heterologous coding sequence are of human origin. In a second preferred embodiment, at least the heterologous coding sequence is from a non-human mammal, preferably a mouse, a rat, or a non-human primate.

In another embodiment, the heterologous coding sequence encodes a marker protein. In another embodiment, the marker protein is a luciferase, a GFP or a beta-galactosidase, or a derivative or variant of one of these, or any other fluorescent or bioluminescent protein. In another embodiment, the heterologous coding sequence encodes a therapeutic protein. In another embodiment, the heterologous coding sequence encodes a functional RNA sequence. In another embodiment, the functional RNA is an RNAi sequence, ribozyme, or antisense sequence.

In another aspect, the present invention provides a method for expressing a protein or RNA in an NK cell of a mammal, the method comprising providing an expression construct comprising a nucleic acid sequence encoding the protein or functional RNA, operably linked to a promoter from a mammalian NCR, introducing the construct into a nonhuman mammal, wherein the RNA or protein is expressed within NK cells of the mammal. In a preferred embodiment, said method is a non-therapeutic method.

In one embodiment, a first and a second (and optionally a third, fourth, fifth, etc.) expression construct are provided and both introduced to a non-human animal, wherein the first and second constructs comprise a heterologous coding sequence encoding a first and second protein. In one embodiment, the first and second protein are not identical. In one embodiment, the first and second protein are proteins known to be expressed on an NK cell. In an exemplary embodiment, the first protein is a protein capable of acting in conjunction with the second protein—for example as a co-receptor to a second receptor.

In one embodiment, the NCR is from a human. In another embodiment, the nonhuman mammal is a mouse. In another embodiment, the NCR is NKp46. In another embodiment, the protein or functional RNA is not expressed in any cell type other than NK cells within the nonhuman mammal. In another embodiment, the protein is a marker protein. In another embodiment, the marker protein is a luciferase, GFP or beta-galactosidase, or any variant or derivative thereof. In another embodiment, the protein or functional RNA affects the activity or proliferation of NK cells within the mammal. In certain embodiments, the functional RNA is an antisense, polynucleotide ribozyme, or RNAi. In another embodiment, the protein is a mutant protein that is associated with a human disorder, and wherein the mutant protein is expressed in the nonhuman mammal to produce an animal model for said human disorder.

In another aspect, the present invention provides a method of specifically marking NK cells within a nonhuman mammal, the method comprising: providing an expression construct comprising a nucleic acid sequence encoding a detectable marker, operably linked to a promoter from a mammalian NCR, introducing the construct into cells of the nonhuman mammal, wherein the detectable marker is expressed within NK cells of the nonhuman mammal. In a preferred embodiment, said method is a non-therapeutic method.

In one embodiment, the nonhuman mammal is a mouse. In another embodiment, the NCR is from a human. In another embodiment, the NCR is NKp46. In another embodiment, the detectable marker is luciferase, GFP or beta-galactosidase. In another aspect, the present invention provides a transgenic nonhuman mammal produced using any of the herein described methods. In another aspect, the present invention provides NK cells isolated from a transgenic nonhuman mammal produced using any of the herein-described methods. In another embodiment, the invention provides transgenic nonhuman mammal comprising a cell comprising an expression construct comprising a nucleic acid sequence encoding the protein or RNA, operably linked to a promoter from a mammalian NCR.

In another aspect, the present invention provides a method for assessing the effect of a compound on NK cells within a nonhuman mammal, the method comprising: providing a nonhuman transgenic mammal produced using any of the herein-described methods; administering the compound to the mammal; and assessing the activity or number of NK cells in the mammal before and after the administration of the compound.

In another aspect, the present invention provides a method for altering NK cell number or activity in a nonhuman mammal, the method comprising: providing an expression construct comprising a nucleic acid sequence encoding a protein or functional RNA capable of modulating the proliferation, survival, or activity of NK cells, operably linked to a mammalian NCR promoter, and introducing the construct into a nonhuman mammal, wherein the protein or functional RNA is expressed within NK cells of the mammal.

In one embodiment, the marker protein is GFP, and the activity or number of marked NK cells is assessed by counting or isolating fluorescent NK cells within said mammal using FACS. In another embodiment, the test compound is a candidate modulator of NK cell activity, and a detection of an increase or decrease in NK cell activity in the presence of said compound indicates that the test compound is a modulator of NK cell activity.

In another embodiment, the invention provides a method for assessing the effect of a test compound on a nonhuman mammal, said method comprising: (a) providing a nonhuman transgenic mammal according to the invention or a nonhuman mammal to which has been administered a cell according to the invention, said mammal expressing a protein in an NK cell; (b) administering to said mammal a test compound capable of binding to or interacting with said protein; and (c) assessing the effect of said test compound on said animal. In a preferred embodiment, said method is a non-therapeutic method.

Kits comprising the expression constructs, cells, and/or nonhuman transgenic mammals are also provided, typically including instructions for their use, e.g. for use in practicing the herein-described methods. In a preferred embodiment, cells are non-human cells. Alternatively, cells can be human non-embryonic cells.

### BRIEF DESCRIPTION OF THE FIGURES

FIG. 1A shows the overall strategy used to isolate and clone the human NKp46 genomic sequence.

FIG. 1B indicates the general location of the primers used within the human NKp46 (NCR1) gene.

FIG. 2 shows the results of PCR performed using NKp46-specific primers on genomic DNA obtained from the tail of three potentially transgenic mice. One mouse (20.45) was positive for human NKp46.

FIG. 3 shows a FACS plot on blood taken from mice 20.44 (littermate control) and 20.45 (hNKp46 transgenic mouse).

FIG. 4 shows the pedigree chart of hNKp46 transgenic mouse colony. Transgenic animals are shaded in gray. FACS histrograms of NKp46 expression on CD3− NK1.1+ cells (spleen from mouse 2, blood from mice 5 and 8) are shown for indicated mice. Dark gray represent hNKp46 “high” mice, light gray represent hNKp46 “low” mice.

FIG. 5 shows expression of hNKp46 on CD3− NK1.1+ lymphocytes in different organs. Open histograms: transgenic mice. Gray histograms: control or littermate mice. Mice 1, 26 and 29 are transgenic. CD3-Alexa 700, NM1.1-PE-Cy7 was used on mice 1 and C57/B6 on a FACScanto. CD3-FITC, NK1.1-APC was used on mice 23, 26, 27 and 29 on a FACScanto.

FIG. 6A shows expression of NKp46 in the different cell subsets defined by CD3 and NK1.1 expression in lymphocytes of the spleen. Open histograms: transgenic mouse 29. Gray histograms: littermate control mouse 27. The gate on the histograms indicates the percentage of NKp46 positive cells for each CD3, NK1.1 subsets. FIG. 6B shows data of FIG. 6A but in liver. FIG. 6C shows expression of NKp46 transgene in NK and NKT cells based on a staining with a CD1-gal-Cer tetramers and NK1.1. NKT cells are defined as NK1.1+CD1 tetramer positive cells. NK cells are defined as negatives for the CD1 tetramer and NK1.1+. Staining is shown on splenic lymphocytes. FIG. 6D shows data of FIG. 6C but in liver.

FIG. 7 shows expression of hNKp46 in various subset of hematopoietic cells. Open histograms: NKp46 transgenic mouse 26. Gray histograms: littermate control mouse 23.

FIG. 8 shows NK receptors expression on CD3-NK1.1+ gated lymphocytes in the spleen. Open histograms: Transgenic mouse 29. Gray histograms: littermate control mouse 27.

FIG. 9 shows the NKp46 transgene is functional in LAK cells at day 6. FIG. 9A shows redirected killing assay: open symbols are transgenic mouse 26; black symbols are littermate control mouse 23; diamond symbol is Daudi only; square symbol is Daudi+anti-NKp30; triangle is Daudi+anti-NKp46. FIG. 9B is as FIG. 9A, but with: open symbols are transgenic mouse 29; black symbols are littermate control mouse 27.

FIG. 9C showns cytotoxicity assays on various tumor cell lines: open bars are transgenic mouse 29; black bars are littermate control mouse 27.

FIG. 9D is a FACS analysis of LAK cells at day 6 from mice 27 (control) and 29 (NKp46Tg)

FIG. 10 shows a comparison of NKp46 and NK1.1 expression levels in different cell types.

FIG. 11 shows transgenic constructs. For the TW4 to TW6 series, each construct can be created to drive the expression of different genes such as Bim, NKp30 or Cre.

FIG. 12 shows transgenic DNA constructs: IRES-gene-of-interest (“Gene X”) fusion sequences with NKp46 homology arms are generated by overlapping PCR. PCR products are then used to generate final transgenic constructs by homologous recombination in E. coli using lambda bacteriophage recombination machinery.

FIG. 13 shows an NK-specific expression vector.

### DETAILED DESCRIPTION OF THE INVENTION

Introduction

The present invention provides novel methods for expressing proteins and RNA sequences specifically in NK cells of mammals. Such methods, and the mammals and cells derived therefrom, are useful for, e.g., studying the role and/or therapeutic utility of modulations in NK cell activity or number in disorders such as tumors, infectious diseases, and autoimmune diseases. In addition, the methods can be used to specifically mark NK cells in nonhuman mammals, e.g. to test the effects of compounds or treatment methods on NK cells. The expression vectors and constructs described herein, cells and nonhuman transgenic mammals comprising them, and kits comprising any of the above, as well as compounds identified using the nonhuman mammals, are also encompassed.

Definitions

As used herein, the following terms have the meanings ascribed to them unless specified otherwise.

As used herein, “NK” cells refers to a sub-population of lymphocytes that is involved in non-conventional immunity. NK cells can be identified by virtue of certain characteristics and biological properties, such as the expression of specific surface antigens including CD56 and/or CD16 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art.

As used herein, the term “nonhuman mammal” refers to any member of the class Mammalia, including Prototheria, Metatheria, and all nonhuman members of the subclass Eutheria, including Insectivora (e.g., moles, shrews), Dermoptera (e.g., flying lemurs), Chiroptera (e.g., bats), Cetacea (e.g., whales), Carnivora (e.g., cats, bears, dogs, otters, seals, sea lions), Tubulidentata (e.g., aardvarks), Proboscidea (e.g., elephants), Hyracoidea (e.g., hyraxes), Primates other than humans (e.g., monkeys, lemurs, bushbabies, aye-ayes, apes), Xenarthra or Edentata (e.g., armadillos, anteaters, sloths), Pholidota (e.g., pangolins), Lagomorpha (e.g. rabbits, hares, pikas), Rodentia (e.g., mice, rats, squirrels, porcupines, beavers, voles, hamsters), Sirenia (e.g., manatees, dugongs), Perissodactyla (e.g., horses, donkeys, zebras, rhinoceroses, tapirs), Artiodactyla (e.g., pronghoms, deer, camels, gnus, goats, giraffes, hippopotami, pigs, peccaries, chevrotains, musk-deer, cows), Scandentia (e.g., tree shrews), and Macroscelidea (e.g., Elephant Shrews). Generally, a “mammal” refers to any animal classified as a mammal, including the above-listed animals and specifically including laboratory, domestic and farm animals, and zoo, sports, or pet animals, such as mouse, rat, rabbit, pig, sheep, goat, cattle and higher primates including humans.

NCR refers to a class of activating receptor proteins, and the genes expressing them, that are specifically expressed in NK cells. Examples of NCRs include NKp30, NKp44, and NKp46 (see, e.g., Lanier (2001) Nat Immunol 2:23-27, Pende et al. (1999) J Exp Med. 190:1505-1516, Cantoni et al. (1999) J Exp Med. 189:787-796, hSivori et al (1997) J. Exp. Med. 186:1129-1136, Pessino et al. (1998) J Exp Med. 188(5):953-60; Mandelboim et al. (2001) Nature 409:1055-1060, the entire disclosures of which are herein incorporated by reference). These receptors are members of the Ig superfamily, and their cross-linking, induced by specific mAbs, leads to a strong NK cell activation resulting in increased intracellular Ca⁺⁺ levels, triggering of cytotoxicity, and lymphokine release. As used herein, NCR can refer to such proteins, genes, or regulatory sequences such as promoters from any organism, including humans, as well as to sequences sharing 70%, 75%, 80%, 85%, 90%, 95%, 96%, 97%, 98%, 99%, or higher amino acid or nucleotide identity or similarity to any NCR from any organism, or to such genes or regulatory sequences which are capable of hybridizing to a gene or regulatory sequence of any NCR from any organism (or to a portion or fragment thereof), particularly when the sequences share one or more properties of a NCR, such as NK cell specific expression and ability to activate NK cells.

The percent identity between two sequences is a function of the number of identical positions shared by the sequences (i.e., % identity number of identical overlapping positions/total number of positions×100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences used. The percent identity between two sequences can be determined using techniques similar to those described above, with or without allowing gaps. In calculating percent identity, typically only exact matches are counted.

As used herein, the term “hybridizes to” describes conditions for hybridization and washing under which nucleotide sequences at least 30% (preferably, 35%, 40%, 45%, 50%, 55%, 60%, 65%, 70%, 75%, 80%, 85%, 90%, 95%, 98% or 99%) identical to each other typically remain hybridized to each other. For example stringent conditions are known to those skilled in the art and can be found in Current Protocols in Molecular Biology, John Wiley & Sons, N.Y. (1989). In one, non-limiting example stringent hybridization conditions are hybridization at 6× sodium chloride/sodium citrate (SSQ at about 45 C, followed by one or more washes in 0.1×SSC, 0.2% SDS at about 68° C. In a preferred, non-limiting example stringent hybridization conditions are hybridization in 6×SSC at about 45 C, followed by one or more washes in 0.2×SSC, 0.1% SDS at 50-65 C (i.e., one or more washes at 50 C, 55 C, 60 C or 65 C). It is understood that the nucleic acids of the invention do not include nucleic acid molecules that hybridize under these conditions solely to a nucleotide sequence consisting of only A or T nucleotides.

The term “expression vector” includes plasmids, cosmids or phages capable of synthesizing the subject NCR promoter-linked RNA or coding sequence carried by the vector. Preferred vectors are those capable of autonomous replication and/expression of nucleic acids to which they are linked. In the present specification, “plasmid” and “vector” are used interchangeably as the plasmid is the most commonly used form of vector.

The term “expression construct” refers to a combination of promoter and operably linked sequence encoding a protein or a functional RNA such as an RNAi sequence, antisense sequence, or ribozyme. An expression construct, whether integrated into a host genome or present on an extra-chromosomal element, has sufficient elements to permit the expression of the RNA or protein when in the proper cell type or under inductive conditions.

As used herein, the term “promoter” refers to non-transcribed DNA sequences upstream of the transcription start site of a gene. As used herein, promoters include both the core elements involved in the initiation of transcription and binding of RNA polymerase and transcription factors, etc., as well as more distant regulatory elements such as enhancers that regulate the specific temporal and spatial regulation of the gene (see, e.g., Molecular Biology of the Gene, 5^{th} edition, Benjamin Cummings/Cold Spring Harbor Laboratory Press). For example, in the case of the NCRs, the “promoter” includes all the sequences sufficient to drive NK cell specific expression in a mammal. “Promoters” can also include modified or isolated forms of the transcriptional regulatory elements of the gene. Such modified forms include rearrangements of the elements, deletions of some elements or extraneous sequences, and insertion of heterologous elements. The modular nature of transcriptional regulatory elements and the absence of position-dependence of the function of some regulatory elements such as enhancers make such modifications possible. Numerous techniques are available for dissecting the regulatory elements of genes to determine their location and function. Such information can be used to direct modification of the elements, if desired. In the case of NCRs, the suitability of modified promoters can be readily assessed, e.g., by detecting the ability of the promoter to drive NK cell specific expression.

An “enhancer” is a type of regulatory element that can increase the efficiency of transcription, regardless of the distance or orientation of the enhancer relative to the start site of transcription.

The term “tissue-specific” or “cell specific,” with respect to a promoter or gene expression, refers to a nucleotide sequence that regulates expression of a selected DNA sequence in specific cells or tissues, such as NK cells. Ideally, tissue specific promoters do not drive any expression in any other tissues, although some expression outside of the tissue is allowed within the scope of the definition, so long that expression is not ubiquitous throughout the organism.

A nucleic acid is said to be “operably linked” when it is placed into a functional relationship with another nucleic acid sequence. In particular, for the purposes of the present invention, a promoter or enhancer is operably linked to a coding sequence if it drives the transcription of the sequence, e.g. in NK cells. Generally, “operably linked” means that the DNA sequences being linked are contiguous. However, enhancers do not have to be contiguous. Linking is accomplished by ligation at convenient restriction sites. If such sites do not exist, the synthetic oligonucleotide adaptors or linkers are used in accordance with conventional practice.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, akyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acids can be either single stranded or double stranded.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “functional RNA” refers to any RNA sequence that has biological activity by itself, i.e. without being translated into protein. Examples of functional RNAs include antisense RNA sequences or RNAi (e.g. which are capable of downregulating the expression of complementary genes), and ribozymes, which are capable of cleaving nucleotide sequences having a specific sequence. See, e.g., Breaker (2004) Nature. 432(7019):838-45; Scanlon (2004) Curr Pharm Biotechnol. 5(5):415-20, the entire disclosures of which are herein incorporated by reference.

The term “transfection” refers to the introduction of a nucleic acid, e.g., an expression vector, into a recipient cell by nucleic acid-mediated gene transfer.

“Transformation”, as used herein, refers to a process in which a cell's genotype is changed as a result of the cellular uptake of exogenous DNA or RNA, and, for example, the transformed cell expresses an RNA (functional RNA or mRNA) operably linked to a heterologous NCR promoter.

As used herein, the term “transgene” refers to a nucleic acid sequence which is partly or entirely heterologous, i.e., foreign, to the transgenic animal or cell into which it is introduced, or, is homologous to or identical to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can be operably linked to one or more transcriptional regulatory sequences and any other nucleic acid, such as introns, that may be necessary for optimal expression of a selected nucleic acid.

The term “transgenic” is used herein as an adjective to describe the property, for example, of an animal or a construct, of harboring a transgene. For instance, as used herein, a “transgenic organism” is any animal, preferably a non-human mammal, in which one or more of the cells of the animal contain heterologous nucleic acid introduced by way of human intervention, such as by trangenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express specific proteins or RNAs in the NK cells. The terms “founder line” and “founder animal” refer to those animals that are the mature product of the embryos to which the transgene was added, i.e., those animals that grew from the embryos into which DNA was inserted, and that were implanted into one or more surrogate hosts. The present invention covers such animals as well as any descendents or progeny carrying the herein-described transgene or expression construct.

As used herein, the expressions “cell,” “cell line,” and “cell culture” are used interchangeably and all such designations include progeny. For the purposes of the present invention, such cells can be derived from a transgenic mammal, or produced directly by transformation of cells with one of the herein-described expression constructs or vectors. The words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as obtained in the originally transformed cell or animal are included. In a particular embodiment, the cell is a non-human cell. In a further embodiment, the cell can be a human non-embryonic cell.

Within the context of this invention, the “activity” of NK cells refers to any biological activity of NK cells, such as their capacity to lyse target cells. For instance, an “active” NK cell is able to kill cells that express an NK activating receptor-ligand and fails to express “self” MHC/HLA antigens (KIR-incompatible cells). Examples of suitable target cells for use in redirected killing assays are P815 and K562 cells, but any of a number of cell types can be used and are well known in the art (see, e.g., Sivori et al. (1997) J. Exp. Med. 186: 1129-1136; Vitale et al. (1998) J. Exp. Med. 187: 2065-2072; Pessino et al. (1998) J. Exp. Med. 188: 953-960; Neri et al. (2001) Clin. Diag. Lab. Immun. 8:1131-1135). “Active” or “activated” (or, conversely, “inactive”) cells can also be identified by any other property or activity known in the art as associated with NK activity, such as cytokine (e.g. IFN-γ and TNF-α) production or increases in free intracellular calcium levels.

As used herein, the term “activating NK receptor” refers to any molecule on the surface of NK cells that, when stimulated, causes a measurable increase in any property or activity known in the art as associated with NK activity, such as cytokine (for example IFN-γ and TNF-α) production, increases in intracellular free calcium levels, the ability to target cells in a redirected killing assay as described, e.g. elsewhere in the present specification, or the ability to stimulate NK cell proliferation. The term “activating NK receptor” includes but is not limited to activating forms or KIR proteins (for example KIR2DS proteins), NCR proteins such as NKp30, NKp44 and NKp46, and NKG2D, IL-2R, IL-12R, IL-15R, IL-18R and IL-21R. Methods of determining whether an NK cell is active or proliferating or not are described in more detail below and are well known to those of skill in the art.

As used herein, the term “inhibiting” or “inhibitory” NK receptor” refers to any molecule on the surface of NK cells that, when stimulated, causes a measurable decrease in any property or activity known in the art as associated with NK activity, such as cytokine (e.g. IFN-γ and TNF-α) production, increases in intracellular free calcium levels, or the ability to lyse target cells in a redirected killing assay as described, e.g. elsewhere in the present specification. Examples of such receptors include KIR2DL1, KIR2DL2/3, KIR2DL4, KIR2DL5A, KIR2DL5B, KIR3DL1, KIR3DL2, KIR3DL3, LILRB1, NKG2A, NKG2C NKG2E and LILRB5. Methods of determining whether an NK cell is active or not are described in more detail below and are well known to those of skill in the art.

The term “receptor” denotes a cell-associated protein that binds to a bioactive molecule termed a “ligand.” This interaction mediates the effect of the ligand on the cell. Receptors can be membrane bound, cytosolic or nuclear; monomeric or multimeric. Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

The terms “isolated” “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The term “biological sample” as used herein includes but is not limited to a biological fluid (for example serum, lymph, blood), cell sample or tissue sample (for example bone marrow).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

A “label” or a “detectable moiety” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide. Preferred labels for the purposes of the present invention include labels that can be detected in living animals, particularly fluorescent labels such as GFP and its derivatives (see, e.g., Trugnan et al. (2004) Med Sci (Paris) 20(11):1027-34; Megason et al. (2003) Mech Dev. 120(11):1407-20; the entire disclosures of which are herein incorporated by reference).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

Expression Vectors Useful for NK Cell Specific Expression in Mammals

The present invention provides and involves the use of expression constructs comprising a promoter from a mammalian NCR, operably linked to a heterologous sequence that codes for a polypeptide or for a functional RNA (e.g. antisense, ribozyme, or RNAi). Preferred is a mammalian NCR (Lanier (2001) Nat Immunol 2:23-27) selected from the group consisting of NKp30 (see, e.g., Pende et al. (1999) J Exp Med. 190:1505-1516), NKp44 (see, e.g., Cantoni et al. (1999) J Exp Med. 189:787-796), and NKp46 (also called NCR1, see, e.g. Sivori et al (1997) J. Exp. Med. 186:1129-1136, Pessino et al. (1998) J Exp Med. 188(5):953-60; Mandelboim et al. (2001) Nature 409:1055-1060). Particularly preferred is NKp46. Also preferred is an NCR from a human, although any promoter from any NCR from any mammal can be used. Sequences, genetic and motif information, homology, and other information, including information about homologs in other organisms about such genes are readily available and can be found, e.g., at Entrez Gene database (Human NKp30 Gene ID: 259197; NKp44 Gene ID: 9436; NKp46 Gene ID: 9437, see also Nucleotide NT_{—}011109 providing the Chromosome 19 contig including the human NKp46 gene, and GeneCard GC19P060109 at the Weizmann Institute of Science).

In a preferred embodiment, the promoter is identical to or shares at least 70%, 75%, 80%, 95%, 90%, 95%, 96%, 97%, 98%, 99%, or more nucleotide sequence identity with the human NKp46 promoter present within SEQ ID NO: 1 or 54, or any fragment or derivative thereof that is capable of driving NK cell specific expression. Preferably the human NKp46 sequence comprises the 400 bp portion upstream of the start codon (ATG) of the NKp46 gene in SEQ ID NO 1 or 54.

Any promoter from any mammalian NCR can be used, as can any part of the promoter, so long as it contains sufficient elements to drive NK cell specific expression. Other elements, e.g. enhancers located downstream of the coding sequence of the gene, can also be used. For example, the Examples section describes the use of a construct that includes 8 kb upstream of the start site of the human NKp46 gene, the coding sequence of the gene, and 8.6 kb downstream of the stop codon (see, e.g., SEQ ID NO:1 or 54). These sequences from the human NKp46 gene were sufficient to drive NK cell specific expression in mice. Accordingly, for use in the present invention, the same upstream and downstream sequences from the human NKp46 gene could be used, or, preferably, only the upstream 8 kb sequence. Also, a portion of the upstream sequence could be used, e.g. the 6 kb, 4 kb, 2 kb, 1 kb or 400 bp upstream of the start site. Also, individual fragments (corresponding to discrete functional domains, such as enhancers, that are capable of driving NK cell specific expression) from within the 8 kb region, and possibly the downstream region, can be used either alone or in combination. Such functional elements can be readily identified using standard methods. One of skill in the art could readily examine an NCR promoter (e.g. by deleting portions of the entire promoter, or isolating individual fragments from within the promoter, and testing them in transcription assays in vitro or in vivo) from any mammal to identify elements (or combinations of elements) that can be used in the present invention. It will be appreciated that derivatives, homologs, and fragments of any NCR promoters or promoter elements can be used, so long as they are capable of driving NK cell specific expression in mammals.

Construction of transgenes, expression constructs, and the like can be accomplished using any suitable genetic engineering techniques, including, inter alia, restriction endonuclease digestion, ligation, transformation, plasmid purification, and DNA sequencing. Such techniques are well known in the art and are described, e.g., in Sambrook et al., Molecular Cloning: A Laboratory Manual (Cold Spring Harbor Laboratory, N.Y., (1989)).

The NCR-derived promoter can be operably linked to any sequence that one might want to express specifically in NK cells, for example a cDNA or a genomic DNA sequence. Several non-limiting examples are further provided below.

The expression vectors used in the present invention can also contain one or more other elements useful for optimizing expression of the transgene in the host animal. For example, the construct may include transcription termination elements, intronic sequences, and polyadenylation signals. In still other embodiments, the transgene construct may include additional elements that facilitate its manipulation in cells (e.g., in bacterial cells) prior to insertion in the intended recipient cell. For instance, the vector may include origin of replication elements for amplification in prokaryotic cells, selectable markers for isolating cells (either bacterial cells or mammalian cells such as ES cells). Selectable marker genes often encode proteins necessary for the survival and/or growth of transfected cells under selective culture conditions and include, for example, genes that i) confer resistance to antibiotics or other toxins, e.g., ampicillin, tetracycline or kanamycin for prokaryotic host cells, and neomycin, hygromycin or methotrexate for mammalian cells; or (ii) complement auxotrophic deficiencies of the cell.

It will be appreciated that the expression vectors described herein are useful both for producing transgenic nonhuman mammals and also for directly transforming NK cells, e.g., ex vivo, or in vitro. Such in vitro or ex vivo transformation can be carried out using any standard method, including by electroporation, microinjection, and calcium phosphate treatment. Method for obtaining NK cells from human and non-human mammals for transforming with the expression vectors are known; exemplary protocols for obtaining mouse and human NK cells are provided in the Section titled “Examples”.

Markers. In one set of embodiments, the sequence codes for a marker protein, such as luciferase, RefFP, GFP or beta-galactosidase (see, e.g., Trugnan et al. (2004) Med Sci (Paris) 20(11):1027-34; Megason et al. (2003) Mech Dev. 120(11):1407-20; the entire disclosures of which are herein incorporated by reference), which would therefore allow the specific identification and monitoring of NK cells in culture, in an animal to which NK cells are administered or infused, or in a transgenic animal. Such cells and animals are useful for, e.g., monitoring the effects of test compounds on NK cells (for example, to screen for compounds capable of modulating NK cell activity or number, for investigating the function of a receptor when expressed on an NK cell, or for testing the effect on NK cells of any candidate therapeutic compound for any human disease or condition).

Sequences affecting NK cell activity. The coding sequence can also encode a protein expected to have a modulating effect on the activity or number of NK cells, e.g. a protein that enhances or inhibits proliferation, growth, or apoptosis of cells, or of NK cell activity (e.g. proteins affecting the expression or activity of NK cell receptors or signal transduction molecules). These can be useful, for example, in order to develop animal models for disorders involving increased or decreased NK cell activity or number. Also, in certain embodiments, a gene will be introduced into NK cells to test its effect on NK cell activity, number, or any other property. Such genes could be candidate modulators of NK cell activity or number, for example, or, alternatively, could be a mutated version of a gene normally expressed in NK cells. In one embodiment, a version of a gene that is normally expressed in NK cells and that contains a mutation associated with a human disorder is introduced into a nonhuman mammal in order to generate an animal model for the human disorder. Generally, however, it will be appreciated that any coding sequence that affects any property of NK cells can be used.

In a preferred example, the coding sequence encodes a receptor. Examples include but are not limited to receptors known to be expressed on B cells, T cells, dendritic cells, NK.T cells, gamma-delta T cells, NK cells, etc. Preferably the receptor is a human receptor, a non-human primate receptor (for example a Rhesus monkey (Macacus mulatta), African green monkey (Chlorocebus aethiops), Marmoset (Callithrix jacchus), Saïmiri (Saimiri sciureus), cynomolgus (Macacus fascicularis), and Baboon (Papio hamadryas), or a rodent receptor (for example, mouse, rat, hamster, etc.). In another preferred embodiment, the coding sequence encodes an NK cell receptor, preferably a receptor known to be expressed by NK cells. The methods of the invention will be useful to permit the expression of such proteins specifically in NK cells, and/or in the desired fashion in terms of time and level (some NK cell receptors are otherwise expressed at low levels, or only upon induction) and/or in the desired host species or cell type or subtype, which may or may not be different from the host species or cell type or subtype in which the protein is known to be normally expressed.

NK cell receptors. A number of preferred receptors that the coding sequence may encode are provided as follows by way of example. NK receptors include but are not limited to Ig superfamily receptors and CLTR superfamily receptors. Examples of Ig superfamily receptors are CD2, CD16, CD69, DNAX accessory molecule-1 (DNAM-1), 2B4, NK1.1; killer immunoglobulin (Ig)-like inhibitory receptors (KIRs); ILTs/LIRs; and NCRs such as NKp44, NKp46, and NKp30. Examples of CLTR superfamily receptors are NKRP-1, CD69; CD94/NKG2A, CD94/NKG2C and CD94/NKG2E heterodimers, NKG2D homodimer, and in mice, Ly49 molecules. Another example is the lectin-like NK cell receptor LLT1. Included also generally are inhibitory or activatory NK receptor comprises one or a plurality of immunoreceptor tyrosine-based inhibitory motif (ITIM). ITIMs are generally found on inhibitory receptors, but in some cases also on activating NK receptors and pathways. In one aspect, an ITIM has the consensus sequence psi-x-Y-x-x-psi, where psi represents amino acids with nonpolar side chains. In another embodiment, the NK receptor has an ITIM having the consensus sequence V/I/L/SxYxxL/V/I/S. In yet another embodiment, the NK cell receptor has an ITIM that has the restricted consensus sequence V/I/xYxxL/V or I/V/LxYxxL/V. In other preferred aspects, an inhibitory or activatory NK receptor is capable of interacting with an HLA/MHC molecule, preferably an MHC-I molecule. Also envisioned is the expression of NKRP1A, Nkrp1f and Nkrp1d proteins, and 4Ig-B7-H3 (co-pending provisional U.S. patent application No. 60/598,727 by Moretta et al, and Steinberger et al, (2004) J. Immunol. 172:2352-2359, the disclosures of which are incorporated herein by reference in their entireties).

In one embodiment, the coding sequence encodes a KIR protein. KIR proteins can have forms that are either activatory or inhibitory. Structure and function of KIR receptors is further described in Carrington and Norman (The KIR Gene Cluster, May 3, 2003, available at: world wide website ncbi.nlm.nih.gov/books); Farag et al., Expert Opin. Biol. Ther., 3(2):237-250 (2003); and Biassoni et al., J. Cell. Mol. Med., 7(4):376-387 (2003), the disclosures of which are incorporated herein by reference. Irrespective of the number of Ig subunits, the cytoplasmic domain of KIR are either long (designated “L”) or short (“S”). KIR with long cytoplasmic domains are inhibitory by virtue of the immunoreceptor tyrosine-based inhibition motifs (ITIMs) present in their cytoplasmic domains. Short-tailed KIR transmit activating signals through their interaction with the adaptor molecule, DAP-12 (DNAX activation protein of 12 kD; this molecule is also known as killer cell activating receptor-associated protein or KARAP), which contains immunoreceptor tyrosine-based activation motifs (ITAMs). ITIMs and ITAMs are characteristic of several immunologically important receptors, such as CD5, CD22 and FcgammaRII. KIRs also may be referred to by various other names such as CD158e1, CD158k, CD158z, p58 KIR CD158e1 (p70), CD244, etc.). Any allele or other variant of a KIR may be encoded. Examples include KIR2DL1, −2 and −3, KIR3DL1, KIR3DL2, NKG2A and NKG2C. In other aspects, the KIR is a KIR selected from Table 1 below, the disclosures of the records corresponding to the listed Genbank Accession numbers being incorporated here by reference in their entireties.

Nucleic acids and/or polynucleotides corresponding to the accession numbers listed in Table 1 are disclosed herein beginning at about page 46. Where only a polypeptide sequence is disclosed by a given accession number, any polynucleotide sequence that would encode the polypeptide of the accession number, based upon the redundancy of the genetic code, is contemplated for use in the subject invention.

TABLE 1
Additional human KIR and other NK cell receptors.

| Name | Accession No. |
|---|---|
| KIR2DL4 | X97229 |
| KIR2DL5A | AF217485 |
| KIR2DL5B | AF217486 |
| KIR2DS1 | X89892 |
| KIR2DS2 | L76667 |
| KIR2DS3 | L76670 |
| KIR2DS4 | L76671 |
| KIR2DS5 | L76672 |
| KIR2DP1 | AF204908 |
| KIR3DL1 | L41269 |
| KIR3DL2 | L41270 |
| KIR3DL3 | AF352324 |
| KIR3DS1 | L76661 |
| KIR3DP1 | AF204919 |
| NKG2A | AAB17133 |
| NKG2C | CAA04922 |
| NKG2D | CAA04925 |
| NKG2E | CAA04923 |
| NKG2F | CAA04924 |
| NKp30 | NP_667341 |
| NKp44 | CAB39168 |
| NKp46 | NP_004820 |
| KIR2DL1 | NP_055033 |
| KIR2DL2 | NP_055034 |
| KIR2DL3 | NP_056952 |
| KIR3DL2 | NP_006728 |
| LILRA1 | NP_006854 |
| LILRA2 | AAH27916 |
| LILR3 | AAM18035 |
| LILR3 | AAM18036 |
| LILR3 | AAM18037 |
| LILR3 | AAM18038 |
| LILR3 | AAM18039 |
| LILR3 | AAM18040 |
| LILR3 | AAM18041 |
| LILR3 | AAM18042 |
| LILR4 | NP_036408 |
| LILR5 | NP_077293 |
| ILT8 | AAD02204 |
| ILT10 | AAC99762 |
| ILT11 | AAK52451 |
| ILT11 | AAF73849 |
| TREM-1 | NP_061113 |
| TREM-2 | NP_061838 |
| TREM-LIKE 4 | NP_937796 |
| CD16 | AAH17865 |
| KIRDL1 | NM_014218 |
| KIRDL2 | NM_014219 |
| KIR2DL3 | AAH50730 |
| KIR2DL3 | AAH32422 |
| KIR2DL3 | BC050730 |
| MAFA | AF097358 |
| MAFA | AAC34731 |

In a preferred example, the coding sequence encodes a receptor selected from the group consisting of KIR2DL1, KIR2DL2 or KIR2DL3 (Accession numbers NP_{—}055033 (amino acid) and NM_{—}014218 (mRNA) for KIR2DL1; NP_{—}055034 and NM_{—}014219 for KIR2DL2; and AAH50730/AAH32422 and BC050730 for KIR2DL3; see also PCT patent application no. WO 2005/003172; the disclosure of all of which references are incorporated here by reference). KIR receptors having two Ig domains (KIR2D) identify HLA-C allotypes: KIR2DL2 (formerly designated p58.1) or the closely related gene product KIR2DL3 recognizes an epitope shared by group 2 HLA-C allotypes (Cw1, 3, 7, and 8), whereas KIR2DL1 (p58.2) recognizes an epitope shared by the reciprocal group 1 HLA-C allotypes (Cw2, 4, 5, and 6). The recognition by KIR2DL1 is dictated by the presence of a Lys residue at position 80 of HLA-C alleles. KIR2DL2 and KIR2DL3 recognition is dictated by the presence of a Asn residue at position 80. Importantly the great majority of HLA-C alleles have either an Asn or a Lys residue at position 80. One KIR with three Ig domains, KIR3DL1 (p70), recognizes an epitope shared by HLA-Bw4 alleles. Finally, a homodimer of molecules with three Ig domains KIR3DL2 (p140) recognizes HLA-A3 and -A11. Preferably the coding sequence encoding a KIR encodes at least the portion of the KIR required for recognition of a HLA ligand.

In other embodiments, the coding sequence encodes a receptor having an immunoreceptor tyrosine-based inhibitory motif (ITIM) in its cytoplasmic tail. The ITIM motif enables the inhibitory receptor to interact with and activate SHP-1 and SHP-2 phosphatases. In another aspect the coding sequence encodes an activating receptor of a NK cell which associates noncovalently with dimers of accessory chain(s), preferably a chain selected from the group consisting of TCR-ξ, FcεRI-γ, DAP12 and DAP10, or an activating receptor of a NK cell which contain the immunoreceptor tyrosine-based activation motif or a YxxM motif for positive signaling. The immunoreceptor tyrosine-based switch motif (ITSM; T/SxYxxV/I) has been shown to exist in several human immunoreceptors, including 2B4, signaling lymphocyte activation molecule (SLAM), CD84, and Ly-9 (Sayos, et al, 2001 Blood 97:3867, Howie et al, 2002 Blood 99:957). Preferably the NK cell receptor is 2B4 (CD244) or NTBA (PCT/EP02/07945 filed 17 Jul. 2002 by Biassoni et al).

In another embodiment of the present invention, the coding sequence encodes an activatory NK receptor. Any activating receptor may be suitable for use in the methods, e.g. NKp30 (see, e.g., PCT WO 01/36630, the disclosure of which is herein incorporated by reference in its entirety), NKp44 (see, e.g., Vitale et al. (1998) J. Exp. Med. 187:2065-2072, the disclosure of which is herein incorporated by reference in its entirety), NKp46 (see, e.g., Sivori et al. (1997) J. Exp. Med. 186:1129-1136; Pessino et al. (1998) J. Exp. Med. 188:953-960; the disclosures of which are herein incorporated by reference in their entireties), NKG2D (see, e.g., OMIM 602893), IL-2R, IL-12R, IL-15R, IL-18R, IL-21R, or an activatory KIR receptor, for example KIR2DS1 (CD158h, accession no. X89892), KIR2DS2 (CD158j, accession no. L76667), KIR2DS3 (nkat7 accession no. L76670), KIR2DS4 (CD158i, accession no. L76671) and KIR2DS5 (CD158g, accession no. L76672). (Carrington and Norman, The KIR Gene Cluster, May 3, 2003, available at: World Wide Web site ncbi.nlm.nih.gov/books), or any other receptor present on a substantial fraction of NK cells, and whose activation leads to the activation or proliferation of the cell. In other examples, the coding sequence encodes an activatory receptor is selected from the group consisting of CD2, BY55 (CD160), NKRP-1 (CD161), FcgammaRIIIa (CD16), CD69, DNAM-1 (also CD226 or DNAX accessory molecule-1), CD31 and pen-5 (CD162R) comprised of a glycoprotein pair called PEN5α and PEN5β (U.S. Pat. No. 6,194,549). In another preferred embodiment the coding sequence encodes a receptor of the CLTR family including for example NKRP-1, CD69, NKG2C and NKG2E. In another embodiment the coding sequence encodes a receptor of the integrin protein superfamily including but not limited to LFA-1 or VLA-4.

Activatory receptors can either directly transduce activating signals or can act in connection with adaptor molecules or other receptors. In some aspects, the coding sequence encodes an adaptor molecule or other receptor.

In another embodiment, the sequence encodes an activatory leukocyte immunoglobulin-like receptor (LILR, or immunoglobulin-like transcript (ILT), a leukocyte inhibitory receptor (LIR) or a macrophage inhibitory receptor (MIR)) found on NK cells, preferably a receptor selected from the group consisting of LILRA1 (LIR-6), LILRA2 (ILT1, LIR-7), LILRA3 (ILT6, LIR-4), LILRA4 (ILT7), LILR5B (LIR-8, ILT8, ILT9, ILT10, ILT11).

The LILRs are also known as ILT (immunoglobulin like transcript), LIR (leukocyte inhibitory receptor), MIR (macrophage inhibitory receptor) and HM transcripts. LILR is the more recently derived and HUGO-endorsed nomenclature (World Wide Web site gent.ucl.ac.uk/nomenclature/genefamily/lilr.html). These receptors are described, including references and their expression in Carrington and Norman, (The KIR Gene Cluster, May 3, 2003, available at: World Wide Web site ncbi.nlm.tih.gov/books), see in particular Table 1 of Carrington and Norman. LILRs are known to interact with HLA class I molecules, are expressed by a range of immunologically active cells, including NK, and have the potential to regulate the immune response through inhibition or activation of cytolytic activity. LILRs have either two or four extracellular Ig domains and a long or short cytoplasmic tail. Long cytoplasmic tails contain up to four immunoreceptor tyrosine-based inhibitory molecules (ITIM) and therefore have the capacity to inhibit cellular activity. LILR with short cytoplasmic domains can associate with molecules containing ITAMs and contribute to cell activation. LILRA3 (ILT6) is unique in that it does not possess a cytoplasmic domain, and may be secreted.

In another embodiment, the compound, preferably an antibody or a fragment thereof, blocks and/or neutralizes the inhibitory signal of mast cell function-associated antigen (MAFA), a type II membranal glycoprotein (Accession nos. AF097358 and AAC34731). MAFA comprises a C-type lectin domain and an immunoreceptor tyrosine-based inhibitory motif (ITIM), located in the extracellular and intracellular domains of MAFA, respectively. Human and mouse homologues of MAFA (first identified on rat mucosal-type mast cells) are expressed only or also by NK and T-cells, although they are thought to play different roles on these cells. MAFA clustering by its specific antibody mAb G63 has been previously shown to cause dose-dependent inhibition of the secretory response of these cells to the FcepsilonRI stimulus. For a review see Abramson et al, Mol Immunol. 2002 September;38(16-18):1307-13. MAFA is recognized by monoclonal antibody G63 described in Abramson et al, 2004 (Immunol Lett. 92(1-2):179-84); MAFA is also known as KLRG1 in mouse, the mouse receptor being recognized by antibody 2F1 described in Raulet et al, 2000 (Eur. J. Immunol. 30(3):920-930).

In yet other embodiments, the methods for expressing a protein or RNA may comprise providing two or more coding sequences (e.g. at least a first and a second coding sequence) each operably linked to a NCR. These coding sequences may be operably linked to the same or to a different NCR, to an identical, similar NCR or different NCR, and/or may be on the same or a different expression construct. In one example the first protein is a CD94 protein and the second protein is an NKG2 protein (e.g. to express CD94/NKG2A, CD94/NKG2C and CD94/NKG2E heterodimers). In another exemplary embodiment, the first protein is a receptor protein (for example an ITIM containing protein such as a Ig superfamily or CLTR superfamily protein) and the second protein is an signal transduction protein such as DAP12. Other proteins that can be expressed according to the invention are TREM receptors (e.g. TREM-1, TREM-2; TREM-4). (Bouchon et al. J. Immunol. 164 (10), 4991-4995 (2000); Colonna, Nat Rev Immunol. 2003 June;3(6):445-53

Creating insertions/deletions in NK cells' DNA. The invention can also be used to modify the expression of a selected target gene in an NK cell, for example to insert or delete a coding sequence. These methods can be particularly useful in creating a deficiency for a target gene in an NK cell. For example, DNA constructs allowing homologous recombination such as the Cre-LoxP system can be used in connection with the invention to selectively insert a nucleotide sequence at a desired location in the genome of an NK cell. Preferably, a coding sequence encoding a Cre polypeptide is operably linked to an NCR, such that Cre is expressed in an NK cell.

The Cre-loxP system used in combination with a homologous recombination technique has been first described by Gu et al. (Cell. (1993) 73(6):1155-64); and Gu et al. (Science (1994) 265(5168):103-6), which disclosures are hereby incorporated by reference in their entireties. These DNA constructs make use of the site specific recombination system of the PI phage. The PI phage possesses a recombinase called Cre which interacts specifically with a 34 base pairs loxP site. The loxP site is composed of two palindromic sequences of 13 bp separated by a 8 hp conserved sequence (Hoess et al., (1986) Nucleic Acids Res. 14:2287), which disclosure is hereby incorporated by reference in its entirety. The recombination by the Cre enzyme between two loxP sites having an identical orientation leads to the deletion of the DNA fragment. Briefly, a nucleotide sequence of interest to be inserted in a targeted location of the genome harbors at least two loxP sites in the same orientation and located at the respective ends of a nucleotide sequence to be excised from the recombinant genome. The excision event requires the presence of the recombinase (Cre) enzyme within the nucleus of the recombinant cell host. The recombinase enzyme may be brought at the desired time either by transfecting the cell host with a vector comprising the Cre coding sequence operably linked to a promoter functional in the recombinant cell host (in this case preferably a promoter from an NCR), which promoter being optionally inducible, said vector being introduced in the recombinant cell host, such as described by Gu et al (1993) and Sauer et al ((1988) Proc. Natl. Acad. Sci. U.S.A. 85:5166), which disclosures are hereby incorporated by reference in their entireties; introducing in the genome of the cell host a polynucleotide comprising the Cre coding sequence operably linked to a promoter functional in the recombinant cell host, which promoter is optionally inducible, and said polynucleotide being inserted in the genome of the cell host either by a random insertion event or an homologous recombination event, such as described by Gu et al (1994). The invention therefore encompasses an NK cell comprising a deletion in a gene of interest.

Models of selective NK deficiency. The invention can also be used to selectively create deficiencies in NK cells. This can be useful both for in vitro and in vivo uses. Generally, a sequence capable, when expressed, of inhibiting an activity of an NK cell or preferably leading directly or indirectly to the death of an NK cell can be used according to the methods of the invention (e.g. such sequence is operably linked to a promoter from an NCR). These methods can then be used for example to provide a non-human animal devoid of its NK cells (e.g. a model of NK cell deficiency). A number of suitable sequences leading to death of NK cells are available, for example. In one example, the coding sequence operably linked to a promoter from an NCR encodes a pro-apoptotic protein. A number of proteins are known to facilitate the physiological process of cell death, and any one of these appropriate for expression by an NK cell can be used. For example, the pro-apoptotic protein may be a member of the Bcl-2 family or a protein that binds to it such as the BH3-motif-containing protein called Bim (O'Connor et al. (1998) The EMBO J. 17(2): 384-395, the disclosure of which is incorporated herein by reference). In another example, the coding sequence encodes a receptor rendering the host cell susceptible to treatment with an exogenous molecule. For example, the coding sequence can encode a protein capable of acting as a diphtheria toxin (DT) receptor (see for example Mitamura et al. J. Biol. Chem. (1995) 270(3): 1015-1019) the disclosure of which is incorporated herein by reference). Receptors able to act as DT receptors are not normally expressed by mouse cells, but can function when expressed in mouse cells and lead to cell death. This can be: used in accordance with the present invention to render an NK cell susceptible to the DT toxin. The cell is then brought into contact with DT at the desired moment, following which DT receptor-expressing NK cells are killed.

Functional RMAs. In addition to coding sequences, it is also possible, for identical purposes as those described above, to use NCR promoters to specifically express functional RNA sequences (see, e.g., Breaker (2004) Nature. 432(7019):838-45; Scanlon (2004) Curr. Pharm. Biotechnol. 5(5):415-20, the entire disclosures of which are herein incorporated by reference) in NK cells. For example, ribozymes (see, e.g., Schubert et al. (2004) Curr Drug Targets. 5(8): 667-81; Citti et al. (2005) Curr. Gene Ther. 5(1):11-24, the entire disclosures of which are herein incorporated by reference), antisense RNAs (Tamm (2005) Methods Mol Med. 106:113-34; Phillips (2005) Methods Mol Med. 106:3-10; the entire disclosures of which are herein incorporated by reference), or RNAi (see, e.g., Bantounas et al. (2004) J Mol Endocrinol. 2004 December;33(3):545-57; Campbell et al. (2005) Curr Issues Mol Biol. 7(1):1-6; Nesterova et al. (2004) Curr Drug Targets. 5(8):683-9, Manoharan (Curr Opin Chem Biol. 2004 December;8(6):570-9; Gilmore et al. (2004) J Drug Target. 12(6):315-40; the entire disclosures of which are herein incorporated by reference in their entirety) can be expressed.

Making Transgenic Mammals

Any non-human mammal can be made transgenic using the methods of the present invention, including, inter alia: farm animals such as pigs, goats, sheep, cows, horses, and rabbits; rodents such as rats, guinea pigs, and mice; and non-human primates such as baboons, monkeys, and chimpanzees. Transgenic mice are particularly preferred. The transgenic animals of the present invention all include within a plurality of their cells a transgene of the present invention, which transgene leads to the expression of an encoded protein or functional RNA specifically in NK cells. Various aspects of transgenic animal technology are well known in the art, and are described in detail in literature, such as Hogan et al., Manipulating the Mouse Embryo (Cold Spring Harbor Laboratory, Cold Spring Harbor, N.Y., (1986)), Wall et al., 1992, J. Cell Biochem June: 49(2), 113-20; WO 91/08216 or U.S. Pat. No. 4,736,866, the disclosures of which are hereby incorporated by reference. Animals suitable for transgenic experiments can be obtained from standard commercial sources such as Taconic (Germantown, N.Y.).

It will be appreciated that the present invention covers animals made using the herein-described methods, as well as any descendents of such animals that contain the herein-described expression constructs. Also, transgene-containing cells and cell lines derived from transgenic mammals produced using the present invention, or any cells comprising an expression construct of the present invention, are also within the scope of the invention.

It will also be appreciated that in certain, clinical circumstances, the present inventions can also be used to introduce marker or therapeutic genes or RNAs into human NK cells, e.g. to mark the cells to enable monitoring of NK cells in vivo in a patient during a therapeutic regimen that directly or indirectly affects NK cells, or to specifically alter the activity, number, or another property of NK cells in a patient. For example, in one embodiment, the NK cells could be altered, in vivo or ex vivo, to express a gene that enhances their activity so as to help the patient more effectively defend against tumors or infection. Accordingly, the present invention comprises pharmaceutical compositions comprising a compound identified or validated using any of the herein-described methods, transgenic animals, or transgenic cells, and a pharmaceutically-acceptable carrier.

For making transgenic nonhuman mammals, introduction of the transgene into the embryo may be accomplished by any means, a large number of which are known in the art, so long as it is not destructive to the cell, nuclear membrane or other existing cellular or genetic structures. Such techniques include, but are not limited to, pronuclear microinjection (see, e.g., U.S. Pat. No. 4,873,191, the entire disclosure of which is herein incorporated by reference), retrovirus mediated gene transfer into germ lines (Van der Putten et al. (1985) PNAS 82:6148, the entire disclosure of which is herein incorporated by reference), gene targeting into embryonic stem cells (Thompson et al., (1989) Cell 56:313, the entire disclosure of which is herein incorporated by reference), electroporation of embryos (Lo (1983) Mol. Cell. Biol. 3:1803, the entire disclosure of which is herein incorporated by reference), and transformation of somatic cells, such as cumulus or mammary cells in vitro followed by nuclear transplantation (Wilmut et al. (1987) Nature 385:810-813; and Wakayama et al. (1998) Nature 394:369-374, the entire disclosure of which is herein incorporated by reference). For example, fetal fibroblasts can be genetically modified such that they have integrated an expression construct of the present invention and then fused with enucleated oocytes. After activation of the oocytes, the eggs are cultured to the blastocyst stage. See, for example, Cibelli et al. (1998) Science 280:1256-1258.

In one embodiment, the transgene construct is introduced into a single stage embryo. Generally, the female animals are super-ovulated by hormone treatment, mated and fertilized eggs are recovered. For example, in case of mice, females six weeks of age are induced to super-ovulate with a 5 IU injection (0.1 ml, i.p.) of pregnant mare serum gonadotropin (PMSG; Sigma), followed 48 hours later by a 5 IU injection (0.1 ml, i.p.) of human chorionic gonadotropin (hCG; Sigma). Females are then mated immediately and examined for copulation plugs. Embryos are recovered and placed in Dulbecco's phosphate buffered saline with 0.5% bovine serum albumin (BSA; Sigma), with surrounding cumulus cells removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO2, 95% air until injection.

Fertilized embryos can be incubated in suitable media until the pronuclei appear, at which time the transgene is introduced. In some species such as mice, the male pronucleus is preferred. Alternatively, the exogenous genetic material could be added to the nucleus of the sperm after it has been induced to undergo decondensation.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 10-20 copies of the transgene construct, in order to insure that one copy is functional. Each transgene construct to be inserted into the cell is generally in the linear form since the frequency of recombination is higher with linear molecules of DNA as compared to circular molecules. Linearization is easily accomplished, e.g., by digesting the DNA with a suitable restriction endonuclease.

Following introduction of the transgene, the embryo may be incubated in vitro for varying amounts of time, or reimplanted into the surrogate host, or both. One common method is to incubate the embryos in vitro for about 1-7 days, depending on the species, and then reimplant them into the surrogate host.

The zygote is the best target for introducing the transgene construct by microinjection method. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442, the entire disclosure of which is herein incorporated by reference). As a consequence, all cells of the transgenic animal will carry the incorporated transgene. This will in general also be reflected in the efficient transmission of the transgene to offspring of the founder since 50% of the germ cells will harbor the transgene.

Retroviral infection can also be used to introduce transgenes into a non-human mammal. The developing non-human embryo can be cultured in vitro to the blastocyst stage. During this time, the blastomeres can be targets for retroviral infection (Jaenich (1976) PNAS 73:1260-1264, the entire disclosure of which is herein incorporated by reference). Efficient infection of the blastomeres is obtained by enzymatic treatment to remove the zona pellucida (Manipulating the Mouse Embryo, Hogan eds., Cold Spring Harbor Laboratory Press, Cold Spring Harbor, (1986)). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can also be injected into the blastocoele (Jahner et al. (1982) Nature 298: 623-628). Most of the founders will be mosaic for the transgene since incorporation occurs only in a subset of the cells which formed the transgenic animal. Further, the founder may contain various retroviral insertions of the transgene at different positions in the genome which generally will segregate in the offspring. In addition, it is also possible to introduce transgenes into the germ line by intrauterine retroviral infection of the mid-gestation embryo (Jahner et al. (1982) supra).

Insertion of the transgene construct into the ES cells can also be accomplished using electroporation, in which the ES cells and the transgene construct DNA are exposed to an electric pulse using an electroporation machine and following the manufacturer's guidelines for use. After electroporation, the ES cells are typically allowed to recover under suitable incubation conditions. The cells are then screened for the presence of the transgene.

Pseudopregnant, foster or surrogate mothers are prepared for the purpose of implanting embryos, which have been modified by introducing the transgene. Such foster mothers are typically prepared by mating with vasectomized males of the same species. The stage of the pseudopregnant foster mother is important for successful implantation, and it is species dependent. For mice, this stage is about 2-3 days pseudopregnant. Recipient females are mated at the same time as donor females. Although the following description relates to mice, it can be adapted for any other non-human mammal by those skilled in the art. At the time of embryo transfer, the recipient females are anesthetized with an intra-peritoneal injection of 0.015 ml of 2.5% avertin per gram of body weight. The oviducts are exposed by a single midline dorsal incision. An incision is then made through the body wall directly over the oviduct. The ovarian bursa is then torn with watchmaker's forceps. Embryos to be transferred are placed in DPBS (Dulbecco's phosphate buffered saline) and in the tip of a transfer pipet (about 10 to 12 embryos). The pipette tip is inserted into the infundibulum and the embryos transferred. After the transfer, the incision is closed by two sutures. The number of embryos implanted into a particular host will vary by species, but will usually be comparable to the number of off spring the species naturally produces.

Transgenic offspring of the surrogate host may be screened for the presence and/or expression of the transgene by any suitable method. Offspring that are born to the foster mother may be screened initially for mosaic coat color where a coat color selection strategy has been employed. Alternatively, or additionally, screening can be accomplished by Southern blot or PCR of DNA prepared from tail tissue, using a probe that is complementary to at least a portion of the transgene. It is also desirable to examine NK cells specifically for the expression (and to confirm the absence of expression in other tissues) of the sequence operably linked to the NCR promoter, e.g., by Western blot analysis or immunohistochemistry using an antibody against the protein encoded by the transgene, or by testing for the RNA expression of the transgene using Northern analysis or RT-PCR.

Alternative or additional methods for evaluating the presence of the transgene, particularly within NK cells, include, without limitation, suitable biochemical assays such as enzyme and/or immunological assays, histological stains for particular marker or enzyme activities, flow cytometric analysis, assays measuring NK cell activity, and the like.

While a single copy of the transgene is sufficient to obtain NK cell specific expression in a mammal, in many cases transgenic mammals obtained using the present methods will be mated with other mammals of the same species, either to obtain an increased number of transgenic mammals, or to obtain mammals containing more than one copy of the construct. Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods. Typically, crossing and backcrossing is accomplished by mating siblings or a parental strain with an offspring, depending on the goal of each particular step in the breeding process.

Testing Compounds and Marking Cells using Transgenic Mammals

Transgenic animals expressing functional RNAs or polypeptides specifically in the NK cells are useful for a large number of purposes. Accordingly, in addition to providing expression constructs, transgenic animals, cells derived therefrom, cells comprising the expression constructs, and methods of making any of the same, the present invention provides methods of using transgenic animals, e.g. to evaluate the effect of test compounds or genes on NK cells. Compounds or genes that are identified or validated using the present methods are also encompassed.

In one set of embodiments, transgenic mammals are used to evaluate the effect of a test compound on NK cells. For example, transgenic animals can be made that express a visible marker such as GFP specifically in the NK cells. Test compounds can then be administered to such animals, and the NK cells can be easily detected and/or isolated (e.g. by FACS sorting) in order to easily monitor the effects of the test compound over time. The test compound can be, e.g., a candidate compound for modulating NK cell activity, or, alternatively, a candidate drug with another desired effect but for which it would be useful to measure its effect on NK cells as a way of identifying possible side effects.

In one embodiment, accordingly, the present invention provides a method for evaluating a test compound, the method comprising i) providing a nonhuman mammal comprising an expression construct comprising an NCR promoter operably linked to a nucleotide sequence encoding a detectable marker, ii) administering the test compound to the mammal, and iii) comparing the activity or number of detectably labeled cells in the mammal in the presence or absence of the compound. Similarly, such methods (using animals or cells) can be performed with multiple compounds tested in parallel or in series, e.g. as a method to screen for compounds that modulate the activity or number of NK cells in vivo or in vitro.

In such embodiments, the test compound can be a candidate therapeutic agent for treating a disorder that directly involves NK cells, such as an infection, tumor, or autoimmune disorder. The test compound may, e.g., be designed to enhance or diminish NK cell activity or number, so as to better treat the condition, and the specific marking of the NK cells facilitates their monitoring in live animals in the presence or absence of the test compound. In one embodiment, the test compound is designed to affect NK cell activity or number, and is directed to an NK cell receptor such as a KIR, NCR, or NKG2 receptor.

In another embodiment, the test compound is a candidate drug for treating a condition that does not directly involve NK cells, and the method is used to detect possible side effects of the drug. In such embodiments, the detection of an effect of the compound on the number or activity of NK cells indicates that side effects are present.

In any of these embodiments, the test compounds can include, but are not limited to, proteins, peptides, peptidomimetic drugs, small molecule drugs, chemicals and nucleic acid based agents. Often, a plurality of compounds, or combinations thereof, are run in parallel with different concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Test compounds can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The monitoring of a test compound on marked NK cells can be carried out in any of a variety of ways in live animals. In a preferred embodiment, following the administration of a test compound a blood sample is periodically obtained and examined with respect to the number of NK cells present using, e.g., FACS sorting based on the expression of GFP. Blood samples can also be used to isolate NK cells (also, e.g. using FACS by virtue of GFP expression), e.g. to allow testing of their activity using standard assays or to assess the cells concerning other physical or biological properties.

In another set of embodiments, the present methods can be used to express proteins or RNA sequences specifically in NK cells. In numerous such embodiments, the expressed RNA sequences or proteins could be designed to alter the proliferation, growth, activity, or another property of NK cells. Such applications are useful, e.g. for developing animal models for studying disorders that involve an increase or decrease in NK cell number or activity. In another embodiment, the methods can be used to introduce a human disease-causing mutation in a gene expressed in NK cells into nonhuman mammals to develop an animal model for the disease. The methods can also be used to test possible therapeutic or prophylactic strategies for treating or preventing conditions such as cancer, infections, or autoimmune disorders, e.g., by altering NK cell activity, number, or another NK cell property.

In one preferred example, the present methods are used to express a protein in an NK cell of a transgenic, non-human mammal, and the cell in brought into contact with a compound that binds or otherwise acts on said protein. This bringing into contact can be carried out by administering the compound to the animal. This method can be particularly useful in assessing the effect of the compound (efficacy, safety, etc.) on an animal whose NK cells express the protein. Any suitable parameter or effect can be monitored, a number of which are described herein, including for example physiological changes or well-being of the animal as an indication of safety, or activity of an NK cell from said animal as an indication of efficacy. Moreover, since it is often desired to assess the effect of a compound designed to bind or otherwise act on a human protein, the methods of the invention are particularly advantageous by permitting a human protein to be expressed by an NK cell of a non-human animal. In one example, the protein expressed in an NK cell of the non-human animal is a protein known to be expressed by human NK cells, such as a KIR protein, an NKG2 protein or an NCR protein such as a human KIR2DL1, 2 or 3, NKp30, NKp44, NKp46, NKG2A, NKG2D, IL-2R, IL12R, IL-15R, IL18R or IL21R protein, and the NK cell is brought into contact with a compound which binds or otherwise acts on said human protein. In a preferred embodiment, the compound is an antibody or fragment thereof which binds the respective protein expressed by the NK cell, or an interleukin (e.g. IL2, IL12, IL15, IL18, IL21).

In a specific example, a mouse is made transgenic to express in its NK cells a protein known to be expressed by human NK cells (for example a human KIR2DL1, 2 or 3, NKp30, NKp44, NKp46, NKG2A, NKG2D protein or another protein described herein); an interleukin or an antibody or fragment capable of binding to said human NK cell protein is administered to said mouse. The mouse is observed for any physiological, physical or behavioral changes, including but not limited to activation of NK cells and number of NK cells, reactivity of NK cells toward a target cell, or decreases in numbers of a target cell (e.g. tumor or infected cells). An observation for example that NK cells in said mouse are activated indicates that the interleukin or antibody or fragment can be useful as a medicament; likewise, an observation that NK cells of said mouse are reactive toward a target cells (in the case of tumor or infected cells) indicates that the interleukin or antibody or fragment can be useful as a medicament. The observation that the mouse has an adverse reaction to said interleukin or antibody or fragment indicates that the interleukin or antibody may have toxicity toward a subject to whom it is to be administered.

In another example, the protein expressed by the NK cell of a nonhuman animal using the methods of the invention is a nonhuman protein, including but not limited to a protein known to be expressed by an NK cell, and including but not limited to a protein expressed by the species of said non-human animal. The invention thereby provides reliable and well defined expression of a protein known to be expressed naturally at least in some situations by said NK cells of said animal. In one specific example a transgenic mouse expresses a mouse or other rodent NK protein such as a Ly49 protein and is contacted with a antibody which binds said mouse or other rodent Ly49 protein.

It will be appreciated that these methods can also be practiced using the transformed NK cells as described herein. For example, NK cells transformed according to the methods of the invention can be administered to a nonhuman mammal. In one preferred example, human NK cells are transformed according to the invention and made to express a human protein and are infused to a nonhuman mammal such as an immunodeficient (SCID) mouse.

As discussed above, to generally alter NK cell activity or number, the encoded protein or RNA could be any of a wide variety of genes such as cell cycle, cell growth, or apoptosis related genes, whose expression could enhance or diminish the proliferation or survival of NK cells. Alternatively, the protein or RNA could alter the activity of the NK cells, e.g. by affecting the level or activity of one or more proteins involved NK cell activation, including receptors, signal transduction molecules (e.g., ZAP70, Syk, LAT, SLP76, Shc, Grb2, phospholipase C-gamma enzymes, phosphatidyl-inositol 3-kinases, KARAP/DAP12, CD3zeta), and transcription factors. For example, the protein or RNA could target receptors such as NCRs or KIR receptors, second messengers, or other molecules involved in NK cell activation (see, e.g. Zompi et al. (2005) Immunol Lett. 97(1):31-9; Vivier et al. (2004) Science. 306(5701):1517-9; Hanna et al. (2004) J Immunol. 173(11):6547-63, the entire disclosures of which are herein incorporated by reference). In certain embodiments, only specific elements of the NK cell activation pathway, e.g., specific KIR or NCR receptors, or particular signal transduction pathway elements, are targeted in order to test the precise role of such receptors or signal transduction pathway elements in a disorder or aspect of NK cell physiology.

Transgenic NK cells expressing a heterologous protein or RNA can be evaluated using any of a number of standard assays. For example, NK cells can be evaluated with respect to (i) natural cytotoxicity towards MHC class I negative targets, tumor cells, virally-infected cells, or allogeneic cells (such as PS15, K562 cells, or appropriate tumor cells as disclosed in Sivori et al. (1997) J. Exp. Med. 186: 1129-1136; Vitale et al. (1998) J. Exp. Med. 187: 2065-2072; Pessino et al. (1998) J. Exp. Med. 188: 953-960; Neri et al. (2001) Clin. Diag. Lab. Immun. 8:1131-1135); Pende et al. (1999) J. Exp. Med. 190: 1505-1516),(ii) cytotoxicity towards antibody-coated target cells, (iii) increases in intracytoplasmic Ca2+ concentration as described, e.g., in Sivori et al. (1997) J. Exp. Med. 186:1129-1136, (iv) induction of tyrosine phosphorylation of intracytoplasmic adaptor/effector molecules such as ZAP70, Syk, LAT, SLP76, Shc, Grb2, phospholipase C-gamma enzymes, phosphatidyl-inositol 3-kinases, (v) phosphorylation of receptor-associated transducing chains KARAP/DAP12 or CD3zeta or FcR gamma, (vi) cytokine secretion such as interferon gamma, tumor necrosis factors, IL5, IL10, chemokines (such as MIP-1alpha), TGFbeta, (vii) up- or down-regulation of NK cell surface molecules, such as CD69 and PEN5 respectively.

NK cell activity can also be addressed using a cytokine-release assay, wherein NK cells are incubated with an antibody or ligand to stimulate the NK cells' cytokine production (for example IFN-γ and TNF-α production). In an exemplary protocol, IFN-γ production from PBMC is assessed by cell surface and intracytoplasmic staining and analysis by flow cytometry after 4 days in culture. Briefly, Brefeldin A (Sigma Aldrich) is added at a final concentration of 5 μg/ml for the last 4 hours of culture. The cells are then incubated with anti-CD3 and anti-CD56 mAb prior to permeabilization (IntraPrep™; Beckman Coulter) and staining with PE-anti-IFN-γ or PE-IgG1 (Pharmingen). GM-CSF and IFN-γ production from polyclonal activated NK cells are measured in supernatants using ELISA (GM-CSF: DuoSet Elisa, R&D Systems, Minneapolis, Minn.; IFN-γ: OptE1A set, Pharmingen).

NK cell modulating compounds identified or screened using the herein-described transgenic animals can be assessed in general ways to assess their impact on the overall health and physiological well being of an animal. Safety can be assessed in any of a large variety of ways. For example, the overall toxicity of the compounds can be assessed, by determining the median lethal dose (LD50), typically expressed as milligram per kilogram (mg/kg), in which the value 50 refers to the percentage death among the animals under study. In addition to determining the LD50, safety can also be assessed by monitoring an animals for any detectable response to the administration, including behavioral, physical, or physiological changes as evidenced by heart rate, blood pressure, etc., as well as blood and other laboratory based tests used to examine markers indicative of organ function, such as creatine or BUN for renal function, prothrombin, bilirubin, albumin, or bone marrow various enzymes to determine hepatic function, or others (see, e.g., The Merck Manual of Diagnosis and Therapy, 17^{th} edition, herein incorporated by reference). In one example, effect on is assessed to determine whether NK cell activation has a detrimental effect on precursor cells, for example assessing bone marrow reconstitution. (Koh et. al. (2002) Biol Blood Marrow Transplant. 8(1):17-25)

Test compounds used in the herein-described screening methods, or therapeutic compounds that have been identified or validated using the herein-described methods, can be formulated and administered to mammals, including humans in the case of the therapeutic compounds, using standard methods that are well known to practitioners in the art. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. Such compositions are prepared according to techniques well-known in the art of pharmaceutical formulation.

The compositions of this invention may be administered topically, especially when the target of treatment includes areas or organs readily accessible by topical application, including diseases of the eye, the skin, the joints, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions may be formulated in an ointment such as petrolatum.

The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In another embodiment, compounds can be modified to improve their bioavailability, half life in vivo, etc. For example, compounds can be pegylated, using any of the number of forms of polyethylene glycol and methods of attachment known in the art (see, e.g., Lee et al. (2003) Bioconjug Chem. 14(3): 546-53; Harris et al. (2003) Nat Rev Drug Discov. 2(3):214-21; Deckert et al. (2000) Int J Cancer. 87(3):382-90).

The dose and schedule of administration will be determined by various factors, including the activity of the particular test compound or therapeutic compound, the condition and body weight of the subject. The size of the dose also will be determined by the existence, nature, and extent of any adverse side-effects that may accompany administration in a particular subject. In determining the effective amount of the compound to be administered in a particular patient, a physician may evaluate circulating plasma levels of the compound, compound toxicities, and the production of anti-compound antibodies. In general, the dose equivalent of a compound is from about 1 ng/kg to 10 mg/kg for a typical subject. Administration can be accomplished via single or divided doses. Frequency of administration can also vary, e.g. from 5, 4, 3, 2, or 1 time a day, or 1 time every 2, 3, 4, 5, 10, 15, 20, or more days.

### EXAMPLES

Further aspects and advantages of this invention are disclosed in the following experimental section, which should be regarded as illustrative and not limiting the scope of this application.

### Example I

### Generation of Transgenic Vector

PAC 900MS containing the human genomic sequence for NKp46 and neighboring genes was used to make a transgenic vector using the RecE/T cloning approach (Genebridges GmbH, Dresden, Germany). The protocol was followed according to manufacturers' instructions using the following recombination primers (from Sigma genosys, purification by SDS-PAGE method):

MBL46 − 8kb (SEQ ID NO: 2):
TCAGAAGCCTACAGGACCATCACAGTCTAGGTAACTCTCACAGTGGAAGG
TAAGCCCGTCCCCGTCGACTGAAGACGAAAGGGCCTCGTC
MBR46 + 8.6KB (SEQ ID NO: 3):
ATGGGATCATTCAGTGCTGAAGCCACTCAACCTCCAGGTTTGGGTTAGTA
AAAAGAACTTTGTGTCGACGCGCTAGCGGAGTGTATACTGGC

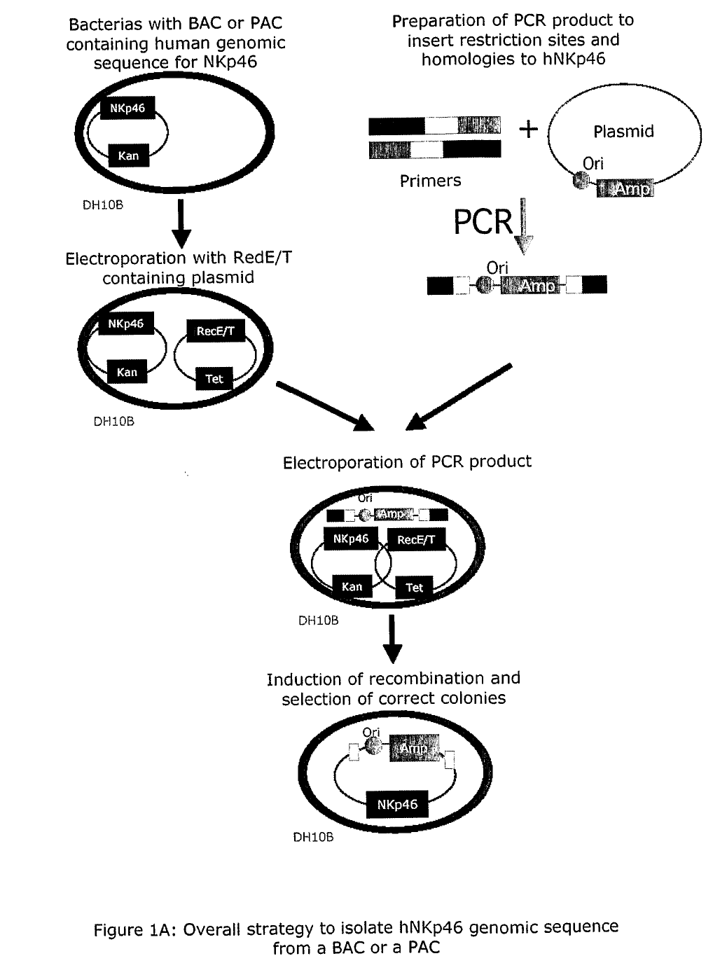

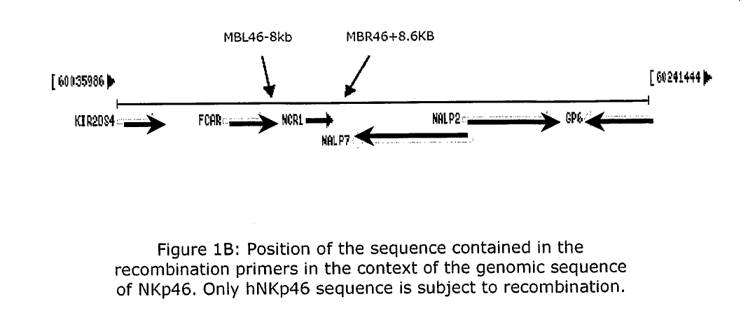

These primers have homology to the pACYC177 vector (21 bp for MBL46-8 kb and 23 bp for MBR46+8.6 KB) followed by a SalI restriction site (6 bp) and human genomic sequence (63 bp corresponding to a sequence 8 kb before the ATG of hNKp46 for MBL46-8 kb and, to a sequence 8.6 kb after the stop codon of hNKp46 for MBR46+8.6 KB). The overall strategy used to generate the transgenic vector is shown in FIG. 1A, and the general location of the primers within the NKp46 genomic region is shown in FIG. 1B.

### Example II

### Inserting hNKp46 Sequence Homologies in pACYC177 Vector

Primers MBL46-8 kb and MBR46+8.6 KB were used to amplify a 2.1 kb DNA fragment containing the origin of replication and the Ampicillin resistance gene of the pACYC177 vector. PCR was performed using 0.5 Taq Platinum high fidelity DNA polymerase (Invitrogen), 1 μl of each primer at 25 μM, 2 μl of 5 mM dNTPs, 5 μl of 10× Platinum buffer, 5 μl of 50 mM MgSO4, 2 μl (10 ng) of HindIII-digested pACYC177, and 33.5 μl ultrapure water. Tubes were heated for 5 min at 94° C., then 30 PCR cycles were performed (45 sec 94° C., 45 sec 62° C., 3 min 68° C.) followed by a 7 min final extension step at 68° C. 5 μl of PCR product was run on an agarose gel to check for the presence of a 2.1 kb band. The remaining 45 μl of the PCR product was precipitated by adding 5 μl 3M Na acetate and 150 μl 100% ethanol. The tube was kept overnight at −20° C. and spun down for 20 minutes at 13,000 rpm at 4° C. in an Eppendorf centrifuge. The pellet was washed with 150 μl 70% ethanol and spun down a second time. The pellet was dried for 10 min at 37° C. and then resuspended in 10 mM Tris-HCl pH 8 at 300 ng/μl.

### Example III

### Transformation of Bacteria Containing PAC 900M8 with Red/ET Recombination Protein Expression Plasmid

Bacteria containing PAC 900M8 were transformed by electroporation according to Genebridges' protocol with pSC101-BAD-gbaAtet plasmid containing Red/ET recombination protein genes. Individual colonies obtained were grown at 30° C. with ampicillin and tetracyclin, and their DNA isolated to check for the presence of the PAC and pSC101-BAD-gbaAtet. Positive colonies were kept for subsequent steps.

### Example IV

### NKp46 Subcloning from PAC 900M8 by RedE/T

Protocol was followed according to manufacturer's instructions. Obtained bacterial colonies were screen by PCR using 0.25 μl Taq DNA polymerase (Invitrogen), 2.5 μl 50 mM MgCl2, 2.5 μl 10× PCR buffer, 1 μl 5 mM dNTPs, 1 μl 10 μM

ScreenFwdpACYC177 primer (AAGTGCCACCTGACGTCTAAGA—SEQ ID NO: 4), 1 μl 10 μM ScreenRvsNKp46-Skb primer (CCATCTGTGCGGGACCCCACTG—SEQ ID NO: 5) and 16.75 μl of ultrapure water. The expected product is 308 bp if recombination occurs. Positive colonies were grown and their DNA checked by restriction enzyme digestion to check the restriction map of the recombined plasmid. Once the restriction map was confirmed, the purified plasmid was electroporated into DH5α Electromax bacteria from Invitrogen following manufacturer's instructions. Bacteria were grown and their DNA was isolated and rechecked by restriction mapping. Recombination regions between pACYC177 plasmid and NKp46 genomic sequence were sequenced. The plasmid obtained has a length of 25,492 bp, shown as SEQ ID NO: 1.

### Example V

### Injection of Transgenic DNA into C57/B6 Mice

Following CIML transgenic facility protocols, the plasmid was prepared with a DNA maxiprep (Qiagen) and then digested with SalI. The digestion product was run on a TAE gel and the 23,444 bp band corresponding to the human hNKp46 genomic sequence was purified using a QIAEX II kit from Qiagen. Further purification was done with an Elutip-d column according to manufacturer's instructions. The final DNA was resuspended in sterile injection buffer (10 mM Tris pH7.4, 1 mM EDTA in water for injection) and the concentration was adjusted to 1 ng/μl.

Injection into fertilized eggs at the pronuclear stage was done by the staff of the CIML transgenic facility.

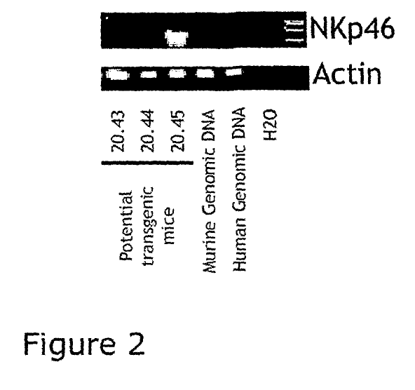

The injected mice were first screened by PCR on genomic DNA taken from the tail. The primers used were MBhNKp46Ex5 (TGCAAGGCTGGTGTTCTCAATGTCG—SEQ ID NO: 6) from exon 5 of human NKp46 and MBhNKp46Ex4 (ACCCACCCTCTCGGTTCATCCTGGA—SEQ ID NO: 7) from exon 4 of the human NKp46 sequence. One mouse (20.45) was positive for human NKp46, as shown in FIG. 2.

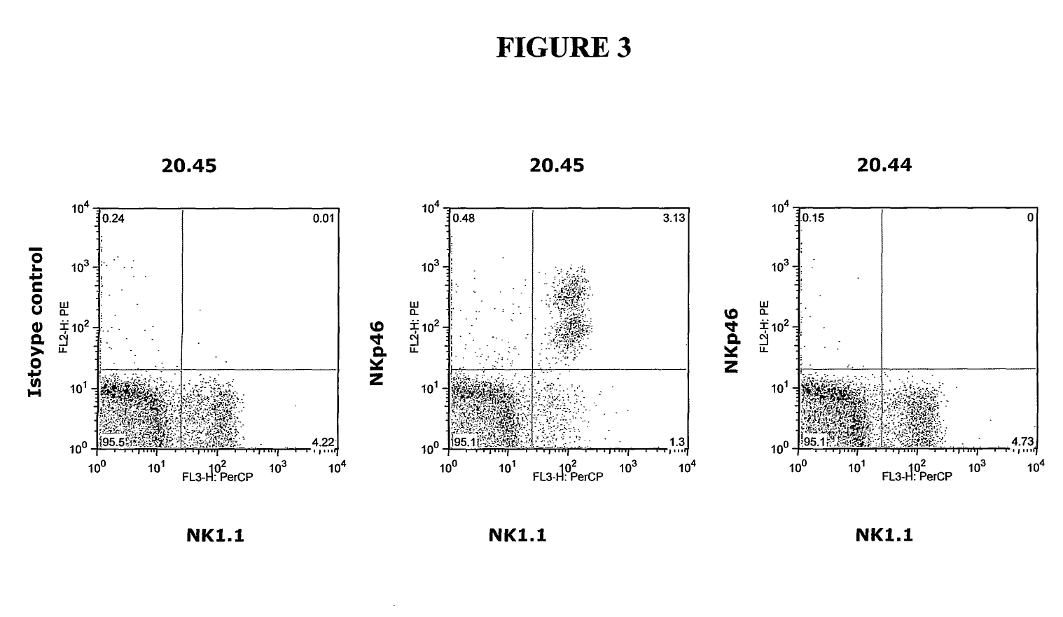

Mouse 20.45 and its littermates was then checked for expression of human NKp46 on blood cells. Expression is restricted to NK1.1+ lymphocytes (FIG. 3).

### Example VI

### Analysis of huNKp46 Transgenic Mice

Materials and Methods:

Cell Preparation:

Mice were sacrificed and the different organs were then harvested in complete medium (RPMI 5% FCS, 10 mM Hepes, 1 mM Na Pyruvate, 100 U/ml Penicillin, 100 μg/ml Streptomycin, 50 μM 2-ME and 2 mM L-glutamnine). For the peritoneal cavity, 10 ml of PBS 1 mM EDTA was injected intraperitoneally in dead mice to flush the cavity. The liquid containing the peritoneal cells was then harvested with a syringe. Liver and Lungs were perfused with PBS 1 mM EDTA to remove blood before harvesting the organ. Cells from spleen, lymph nodes, peritoneal cavity and bone marrow were mashed through a cell strainer and cells suspensions were subjected to Tris Ammonium Chloride red blood cell lysis when necessary, then washed in PBS 2% FCS 1 mM EDTA and counted. Liver and Lungs were homogenised and 1 ml of medium containing 200 U/ml collagenase was added to the cells and incubated for 45 minutes at 37C. Cells were then washed three times in medium. Liver cells were resuspended in 5 ml 80% Percoll in PBS. 5 ml 40% Percoll in PBS were added on top. Lungs cells were resupended in 5 ml of 67.5% Ficoll in PBS. 5 ml of 37.5% Ficoll in PBS were added on top. Tubes were centrifuged for 20 min at 2200 rpm at RT. The rings of cells were harvested and washed in medium. Cells were resuspended in PBS 2% FCS, 1 mM EDTA.

Flow Cytometry:

1 million cells were used per staining whenever possible. Stainings were done in PBS 2% FCS, 1 mM EDTA. Cells were first incubated with 2.4G2 hybridoma supernatant to block Fc receptors except for cells which would be stained for CD16 afterwards. Antibodies were incubated for at least 15 minutes on ice in 50 μl. Two washes were performed between each antibody layer. Events were acquired on a FACS Canto or a FACS Calibur (BD) using either Cellquest or Diva softwares. Analysis of the data was performed with Flowjo software.

LAK:

30 millions splenocytes were put in culture in complete medium supplemented with 4000 U/ml recombinant human IL-2 for 3 days. Non adherents cells were transferred to a new flask and new medium was added complemented with 50 μM 2-ME. Cells were cultured for another 3 days and expression of CD3 and NK1.1 was checked by FACS. LAK cells were immediately used for chromium release assay.

Chromium Release Assay:

1 million indicated target cells were incubated with 100 μl of Chromium-51 (Na₂⁵¹CrO₄, 1 mCi/ml, Perkin Elmer) for 1 hour at 37C. Cells were then washed twice with complete medium and counted. Targets cells were distributed in 96 well plate in duplicates or triplicates with antibodies when indicated. 3000 NK1.1+, NKp46+ LAK cells were then added to the target cells in a final volume of 200 μl. Spontaneous ⁵¹Cr release was obtained from target cells alone. Maximum ⁵¹Cr release was obtained by adding 100 μl 2N HCl to 100 μl of targets cells. Cells were incubated for 4 hours at 37C. Plates were centrifuged for 2 minutes at 600 rpm and 25 μl of culture supernatant were added to 150 μl of Optiphase SuperMix scintillator (Wallac) in a 96 well plate adapted for microbeta counter (Wallac). Cpm for each well were counted and specific lysis was calculated using the formula (((cpm−Min)/(Max−Min))×100) where cpm is the cpm counted in a well, Min is the average of the cpm counted in the spontaneous release triplicate for each target cells, Max is the average of the cpm counted in the maximum release triplicate for each target cell.

Luciferase Assays:

A 400 bp NKp46 promoter was amplified from pACYC177-NKp46 and inserted into the KpNI and XhoI sites of pGL3 reporter vector (Promega). 4 μg of the pGL3-NKp46 or pGL3-promoter (SV40 promoter, Promega) were cotransfected with 1 μg pRLTK control vector (Promega) into NKL, Jurkat or K562 cells (2 millions each) using amaxa nucleofection reagents. 48 h after transfection, Firefly (pGL3) and Renilla (pRLTK) luminescences were measured using dual luciferase assay reagents (Promega) and a Mithras luminescence reader.

Results and Discussion:

1. Analysis of Blood samples and genomic DNA from NKp46Tg mice reveal multiple insertions of the transgene and complex regulation of the transgene expression in cells.

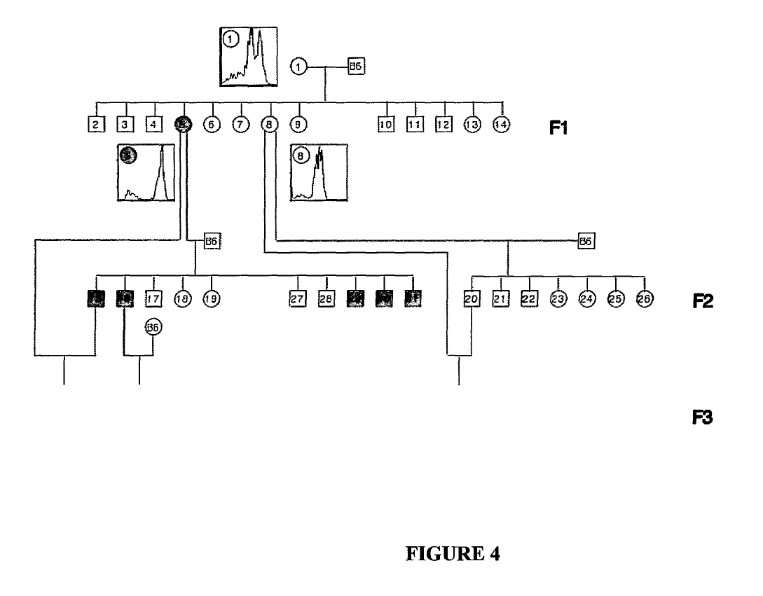

A second peak of fluorescence was observed in NK cells from the first transgenic mouse. This observation implies that any given NK cell express either a high level of huNKp46 or a low level of huNKp46. In the offspring of this mouse, two phenotypes were observed. NK cells of mouse 5 expressed only one major peak of fluorescence with a high MFI. NK cells from mouse 8 also expressed one major peak of fluorescence but with a lower MFI (FIG. 4). Additionally, quantitative PCR from genomic DNA from different mice revealed that mouse 1 has twice the amount of NKp46 tg compared to offspring (table 2). There may thus be two sites of huNKp46 transgene insertion in the genome of mouse 1. These two transgenes behave as independent genes and segregate in the offspring (FIG. 4). It is believed that one transgene may drive low expression of huNKp46 and the other drives high expression of huNKp46. As shown in FIG. 4, mice were bred so as to obtain a colony of huNKp46 with a “high” level of expression and a colony of huNKp46 with a “low” level of expression. Moreover, these observations raise interesting questions about the regulation of huNKp46 expression. Indeed, if both transgenes were expressed in all NK cells, one would expect a broad peak with intermediate intensity of fluorescence. On the contrary, in mouse 1, about 50% of the NK cells expressed low levels of huNKp46 and 50% expressed high levels. This phenomenon might result from the fact that the expression of one transgene in a NK cell blocks the expression of the other. However further breeding and further crossing will be necessary to confirm these observations but this transgenic model may provide an interesting tool to study the regulation of NKp46 gene expression. These observations are also relevant to human as differences in the expression levels of NKp46 are observed in NK cells from different donors (Pende, D. et al. Eur. J. Immunol. 31, 1076-86. (2001).

TABLE 2
Results from Quantitative PCR done to quantify the amount of huNKp46
transgene in genomic DNA from differents mice and comparison to
data obtained from screening the mice with regular PCR.

| | Regular PCR | Normalized values for |
|---|---|---|
| Mouse | on genomic DNA | NKp46 DNA |
| 1 | NKp46 Tg | 54 |
| 20 | NKp46 Tg | 19 |
| 21 | WT | Neg |
| 22 | NKp46 Tg | 21 |
| 23 | WT | Neg |
| 24 | WT | Neg |
| 25 | NKp46 Tg | 25 |
| 26 | NKp46 Tg | 23 |

2. NKp46 transgene is not causing any detectable phenotype.

NKp46 transgenic mice do not display particular phenotypes. Cellularity in tested organs was within normal values. Breeding is normal.

3. NKp46 transgene is expressed on all NK cells.

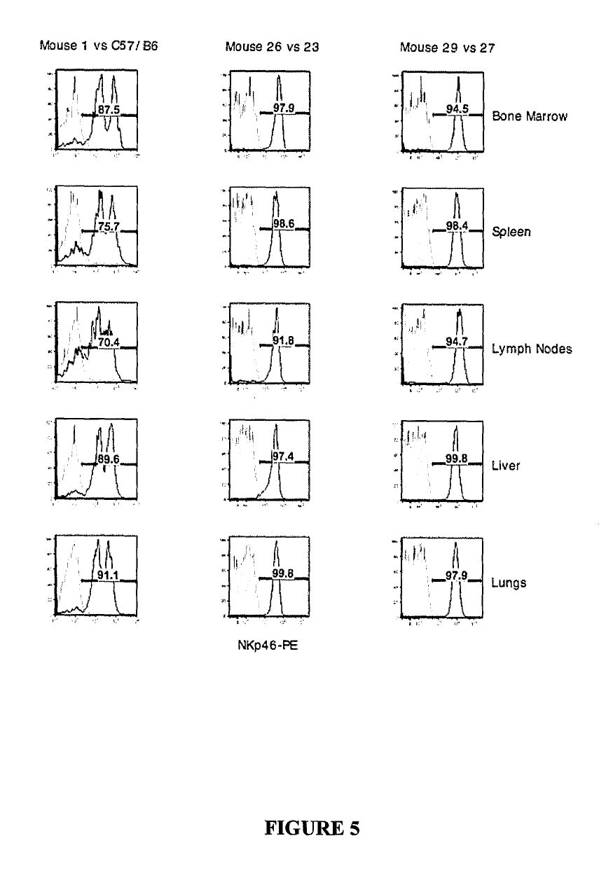

Expression of NKp46 transgene was assessed on CD3-NK1.1+ cells by FACS on cell suspensions obtained from differents organs. As shown in FIG. 5, almost all CD3-NK1.1+ cells express the transgene in mice 26 and 29 in all organs tested. In mouse 1, percentages of positive cells are lower but the combination of antibodies used to discriminate NK cells may not be optimal due to the fluorochromes used (FIG. 5).

4. A minor subset of CD3+ NK1.1+ cells also express NKp46 Tg.

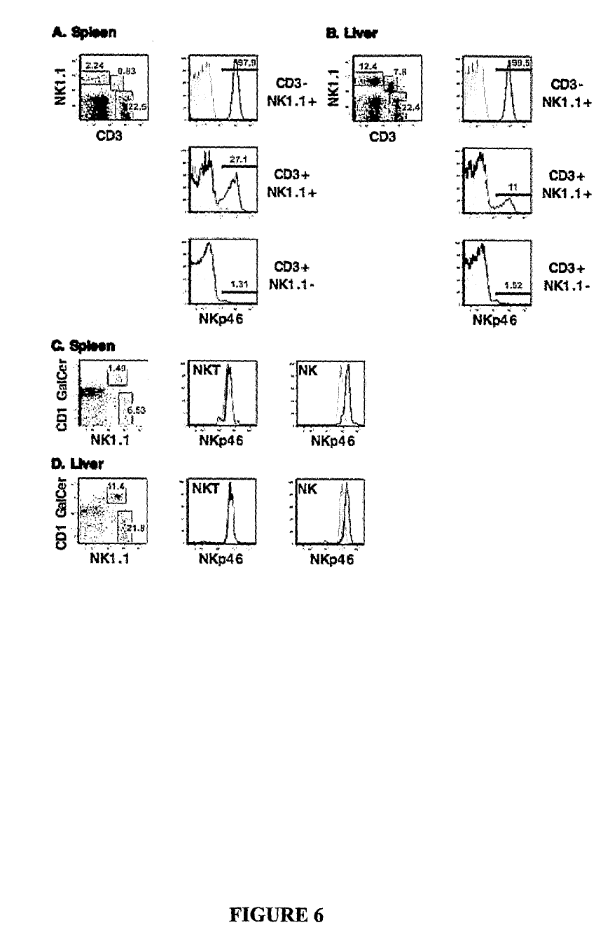

A minor percentage of CD3+ NK1.1+ cells express NKp46. This percentage varies depending upon the organ tested (FIGS. 6A, 6B). These cells are usually NKT cells. However, in the liver where NKT cells are very abundant, the percentage of NKp46+ cells in the CD3+ NK1.1+ cells drops to 11% compared to 27% in the spleen (FIGS. 6A, 6B). To address the expression of NKp46 on NKT cells, lymphocytes from the liver and the spleen were stained with a CD1 tetramer coupled to galactosylceramide. Conventional NKT cells are stained with the tetramer and NK1.1 and are negative for NKp46 (FIGS. 6C and 6D). Moreover, CD3+ NK1.1+ CD4+ NKT cells are also negative for NKp46. Therefore conventional CD1d-dependent NKT cells do not express NKp46 transgene.

5. All other hemapoietic cells do not express NKp46 transgene

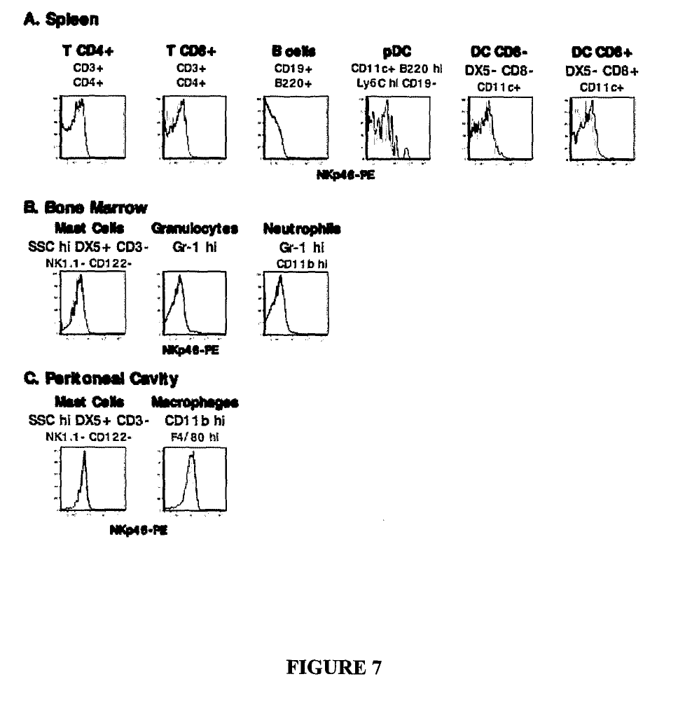

Surface expression of huNKp46 transgene was detected only on NK cells and on a subset of CD3+ NK1.1+ cells as discussed above but expression was not detected on others hematopoietic cells (FIG. 7).

6. Expression of NKp46 transgene does not seem to interfere with expression of other NK receptors.

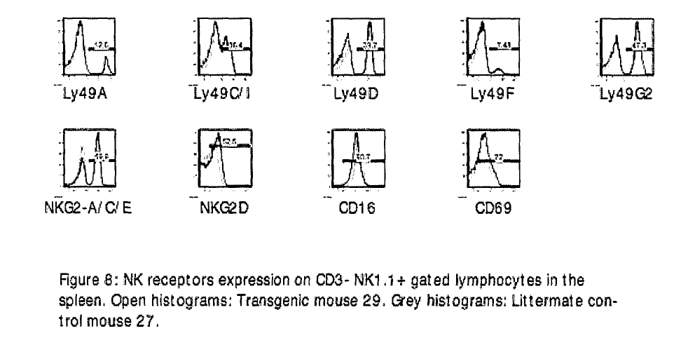

Surface expression of different NK receptors was assessed by FACS on CD3− NK1.1+ gated lymphocytes in the spleen (FIG. 8). There is no particular skewing of the phenotype of NK cells toward expression of a specific pattern of surface receptors. Therefore expression of the NKp46 transgene does not interfere with the normal expression of the others NK surface receptors tested.

7. Human NKp46 transgene is functional and can trigger cytotoxicity by murine NK cells.

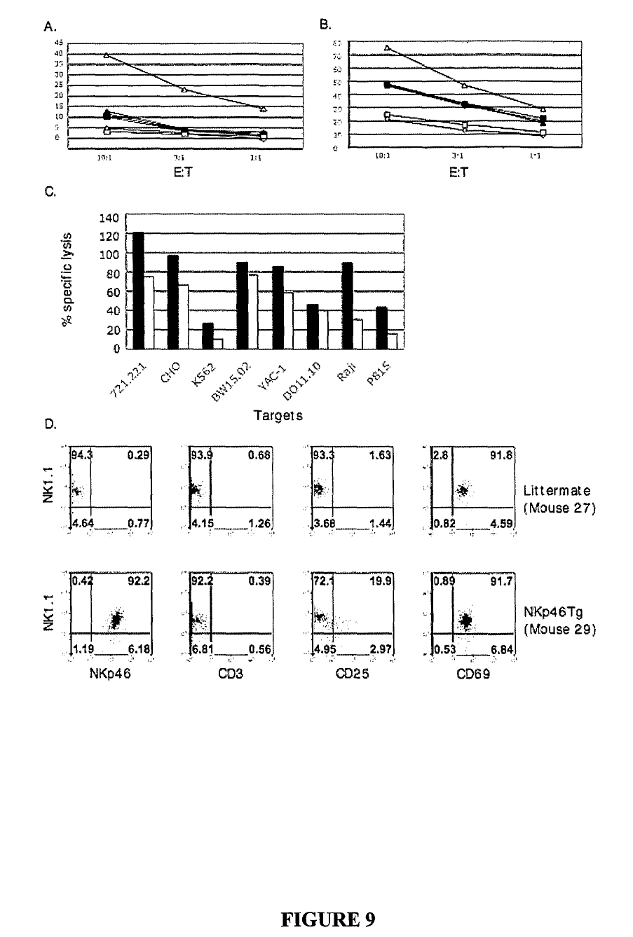

To test the functional capacity of murine NK cells to use the transgenic NKp46 receptor to kill target cells, LAK cells were derived. LAK cells (equivalent to 3000 NK1.1+ NKp46+ cells) were put in contact with various target in standard cytotoxicity assay or standard redirected killing assay measured in 4 h by ⁵¹Cr release (FIG. 9).

In a redirected killing assay, LAK cells from NKp46 transgenic mice were capable of killing Daudi cells coated with Bab281 through their Fc Receptors (FIGS. 9A, 9B). LAK cells from control mice were not capable of killing the Daudi cells under the same conditions. Against various tumor target cell lines, LAK cells from transgenic NKp46 mice are likewise able to kill tumor cells, albeit somewhat less efficiently than control mice, except for BW15.02 and DO11.10 where they were as efficient as their wild-type counterparts (FIG. 9C). BW15.02 is known to express a ligand activating human NK cells through NKp46 (Pessino, A. et al. J. Exp. Med. 188, 953-960 (1998)). FACS analysis of the transgenic LAK revealed that CD25 was upregulated as NK1.1 was downregulated indicating that in the transgenic mice the LAK cells are overactivated. This observation may hint for the presence of an endogenous ligand in the mouse for huNKp46. Therefore NKp46 transgenic LAK cells may have been “exhausted” during the 6 days culture and may have lost some of their killing potential.

In summary, NKp46 transgenic murine NK cells can use the transgene as an activating receptor indicating that the human NKp46 molecule can associate specifically with murine molecules to drive the cytotoxic cellular machinery.

8. A minimal 400 bp NKp46 promoter sequence can be identified and may drive specific expression in murine NK cells in vivo.

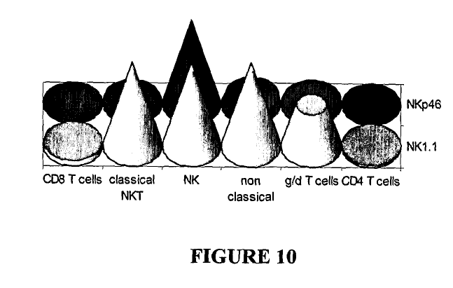

IFN-γ secretion and perform-dependent cytotoxic mechanisms are crucial arms of the immune response against pathogens and in the immunosurveillance of cancer. NK cells, NKT cells, γ/δ T cells and cytotoxic CD8 T cells are capable of exerting these functions. These various cell types differ in the receptors leading to their activation but share the expression of a large set of genes associated with the cytotoxic function. For example, NK1.1, classically used to identify NK cells in C57BL/6 mice, is expressed at various levels by several subsets of cytotoxic lymphocytes. Similarly, antibodies against asialo-GM1, commonly used to deplete NK cells, also deplete various cytotoxic lymphocytes. This phenomenon clearly limits our ability to specifically study the role of NK cells in the immune system and calls for the identification of more specific NK cell markers/tools. As previously described, huNkp46 expression is restricted to NK cells in huNKp46 tg mice (FIG. 10, in comparison with NK1.1). In particular, NKp46 is not expressed in NKT cells. Thus, the regulatory regions contained in the 26 kb Nkp46 sequence are sufficient to drive specific NK cell expression.

This 26 kb NKp46 construction can be used to generate mouse genetic models for the study of NK cell function. As a 26 kb construction is not convenient to manipulate we first sought to determine whether a minimal NKp46 promoter could be identified. Several bioinformatic tools (Pipmaker, Nix, genomatix) were used to identify a 400 bp region directly upstream of the initation codon with a high degree of conservation between rat and human, a putative TATA-box and several putative transcription factor biding sites among which conserved c-ETS and RUNX/AML sites (RUNX3 and ETS1 are required for NK cell development). This 400 bp sequence was amplified and used as a promoter in a reporter assay (pGL3 reporter vector containing the luciferase gene). Different human cell lines were transfected with the reporter construct and luciferase expression was measured 48 h after transfection. The 400 bp NKp46 promoter drives expression of the luciferase in the NKL cell line and at a low level in the Jurkat cell line but not in the K562 erythroid cell line. In comparison, a SV40 promoter drives expression of the reporter gene in all cell lines tested. These results indicate a relative NK cell specificity of this minimal promoter.

### Example VI

### Generation of a Mouse Model of Inducible NK Cell Deletion In Vivo

Methods for dissection of the role of NK cells in vivo, methods which were previously hampered by two major problems: the absence of laboratory animal/mouse models of selective NK cell deficiency and second, the difficulty to track these cells in situ by immunohistochemistry.

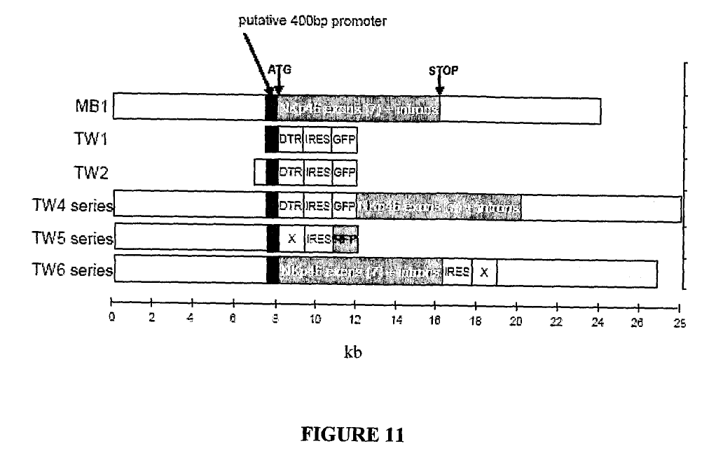

Transgenic mice can be generated whose NK cells specifically express the diphteria toxin receptor and the EGFP genes separated by an internal ribosome entry site (IRES) sequence. In these mice, EGFP expression facilitates NK cell tracking in vivo. Diphteria toxin is a bacterial toxin with two subunits. The first subunit DTA, binds to E2F elongation factors thereby inhibiting translation and triggering cell death. The DTB subunit binds to the surface receptor HBEGF and allows DT internalization. Because of a polymorphism in the extracellular sequence, DTB binds to human HBEGF but not to mouse HBEGF. This property has been successfully used to create several mouse models of cell ablation, where a particular cell type expressing human HBEGF can be killed by injection of small amounts of diphteria toxin. This depletion is transient as diphteria toxin has a short half-life in vivo. Based on results described in the Examples herein, a transgenic construct named TW1 (see FIG. 11 for a summary of all transgenic constructs) was made using the 400 bp NKp46 promoter followed by the Diphteria Toxin receptor (DT-R), an IRES (Internal Ribosome Entry Site) and the EGFP. This transgene was injected in fertilized eggs at pronuclear stage from C57/B10xDBA2 mice. 8 transgenic mice were obtained but only one had detectable EGFP expression in PBMC. This EGFP expression is restricted to NK cells. Thus, the 400 bp NKp46 promoter construction seems to retain the specificity of the full-length promoter. However, it appears to lack enhancer region(s), as only one mouse out of 8 expressed the transgene. This enhancer region seems not to be present in the 1 kb region upstream the ATG initiation codon, as a 1 kb NKp46 promoter also failed to drive expression of the same DTR/IRES/EGFP cassette (construct TW2, FIG. 11).

### Example VII

### Specific Expression of Genes in Murine NK Cells

To generate a mouse model of permanent (as opposed to transient, in Example VI) depletion of NK cells, the proapoptotic Bcl2 member Bim is expressed in NK cells. Bim is known to induce lymphocyte death when over-expressed (Hildeman, D et al Curr Opin Immunol 14, 354-9. (2002)). Thus the expression of Bim can induce NK cell death. Next, human NKp30 is expressed in NK cells. As there is no mouse ortholog of NKp30, this transgenic model should bring valuable information in the function of this receptor. Moreover, this provides the first pre-clinical model for the study of NKp30. Next, the bacterial Cre recombinase gene is expressed in NK cells. By crossing the Cre transgenic mice obtained with available mice containing “floxed” genes, the role of these various genes in NK cell compartment can be examined. Lox sites on each side of the floxed genes are recognized by the Cre recombinase only in NK cells and the gene are removed from the genome of NK cells.

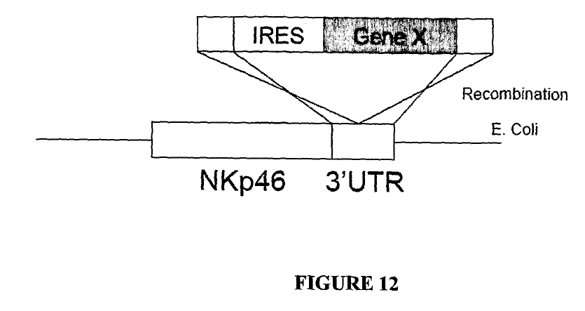

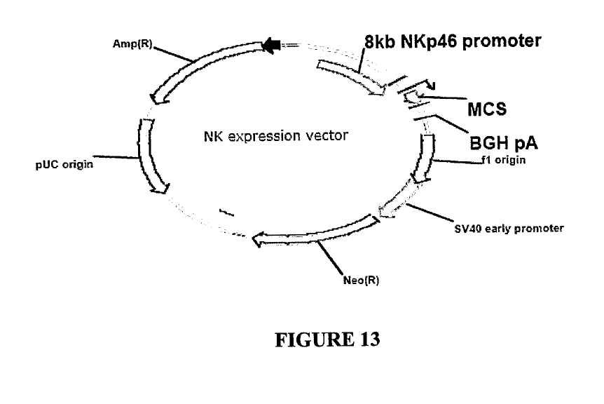

Based on the aforementioned transgenesis attempts, optimal efficient expression of a transgene in NK cells are sought using the transgenic TW4 series (FIG. 11). Two other alternative strategies can also be used to express genes specifically in NK cells. One is to insert an IRES sequence followed by the cDNA sequence of various transgenes in the 3′UTR region of the NKp46 gene, downstream the STOP codon. (FIG. 12 and see also FIG. 11, TW6 series). This classical knock-in strategy has been shown to drive co-expression of both genes on each side of the IRES. Insertion of IRES-gene X downstream NKp46 stop codon will be performed by homologous recombination in E. Coli. DNA constructs will then be injected into ovocytes for the generation of transgenic mice. A second strategy is to generate an NK-specific expression vector where the 8 kb upstream the NKp46 initiation codon will be used as promoter, and where cDNA sequences will be inserted into a multiple cloning site (MCS) located between the NKp46 promoter and a BGH polyA signal (FIG. 13 and FIG. 11, TW5 series). Such a convenient vector can be tested first in transient transfection assays using various cell lines before validation for transgenesis experiments.

### Example VIII

### Purification of Peripheral Blood Lymphoocytes (PBL) and Generation of Polyclonal or Clonal NK Cell Populations

Peripheral blood lymphocytes (PBL) are derived from healthy donors by Ficoll-Hipaque gradients and depletion of plastic-adherent cells. In order to obtain enriched NK cells PBL are incubated with anti-CD3 (JT3A), anti-CD4 (HP2.6) and anti-HLA-DR (D1.12) mAbs (30 min at 4 degrees C.) followed by goat anti-mouse coated Dynabeads (Dynal, Oslo, Norway) (30 min at 4 degrees C.) and imniunomagnetic depletion (Pende et al. (1998) Eur. J. Immunol. 28:2384-2394; Sivori et al. (1997) J. Exp. Med. 186: 1129-1136; Vitale et al. (1998) J. Exp. Med. 187:2065-2072). CD3⁻4⁻DR⁻ cells are used in cytolytic assays or cultured on irradiated feeder cells in the presence of 100 U/ml r1L-2 (Proleukin, Chiron Corp., Emeryville, USA) and 1.5 ng/ml PHA (Gibco Ltd, Paisley, Scotland) in order to obtain polyclonal NK cell populations or, after limiting dilution), NK cell clones (Moretta (1985) Eur. J. Immunol. 151:148-155).

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1-39. (canceled)

40. A method for expressing a protein or functional RNA in an NK cell of a nonhuman mammal, said method comprising:
a) providing an expression construct comprising a promoter from an NCR of a mammal, operably linked to a nucleic acid encoding said protein or functional RNA; and
b) introducing said construct into a nonhuman mammal, wherein said protein or functional RNA is expressed within NK cells of said nonhuman mammal.

41. The method of claim 40, wherein said NCR is a human NCR.

42. The method of claim 40, wherein said nonhuman mammal is a mouse.

43. The method of claim 41, wherein said NCR is NKp46.

44. The method of claim 40, wherein said protein or functional RNA is not expressed in any cell types other than NK cells within said nonhuman mammal.

45. The method of claim 44, wherein said protein is a marker protein.

46. The method of claim 45, further comprising the detection of said marker protein to identify marked NK cells and wherein said marker protein is a GFP, a luciferase or a beta-galactosidase.

47. A composition of matter comprising:
a) a nonhuman transgenic mammal comprising a mammalian NCR promoter operably linked to a nucleic acid sequence encoding a heterologous protein or functional RNA;
b) an isolated NK cell comprising a mammalian NCR promoter operably linked to a nucleic acid sequence encoding a heterologous protein or functional RNA;
c) an expression construct comprising a mammalian NCR promoter operably linked to a nucleic acid sequence encoding a heterologous protein or functional RNA;
d) a nonhuman transgenic mammal comprising a mammalian NKp46 promoter operably linked to a nucleic acid sequence encoding a heterologous protein or functional RNA;
e) an isolated NK cell comprising a mammalian NKp46 promoter operably linked to a nucleic acid sequence encoding a heterologous protein or functional RNA; or
f) an expression construct comprising a mammalian NKp46 promoter operably linked to a nucleic acid sequence encoding a heterologous protein or functional RNA.

48. The composition of matter of claim 47, wherein said NCR is NKp46.

49. The composition of matter of claim 47, wherein said composition of matter is an expression construct and said expression construct further comprises a gene encoding a heterologous protein.

50. The composition of matter of claim 49, wherein the heterologous protein is luciferase, RedFP, GFP or beta-galactosidase.

51. The composition of matter of claim 47, wherein said composition of matter is an expression construct and said expression construct further comprises a gene encoding a heterologous protein and said heterologous protein or functional RNA affects the activity or proliferation of NK cells.

52. The composition of matter of claim 47, wherein said composition of matter is an expression construct and said expression construct further comprises a gene encoding a heterologous protein and said heterologous protein comprises an immunoreceptor tyrosine-based inhibitory motif (ITIM), an immunoreceptor tyrosine-based activation motif (ITAM), a killer Ig-like inhibitor receptor, NKp30, NKp44, a protein of the CLTR superfamily, diphtheria toxin receptor, Bim, Bcl-2 or Cre.

53. The composition of matter of claim 47, wherein said composition of matter is an expression construct and said expression construct further comprises a gene encoding a functional RNA and said functional RNA is an RNAi, antisense, or ribozyme.

54. A method for assessing the effect of a test compound on a nonhuman mammal, said method comprising:
a) providing: i) a nonhuman transgenic mammal comprising a mammalian NCR promoter operably linked to a nucleic acid sequence encoding a heterologous protein or functional RNA; or ii) a nonhuman mammal to which a NK cell comprising a mammalian NCR promoter operably linked to a nucleic acid sequence encoding a heterologous protein or functional RNA has been administered;
b) administering to said mammal a test compound capable of binding to or interacting with said protein; and
c) assessing the effect of said test compound on said animal.

55. The method of claim 54, wherein assessing the effect of said test compound on said animal comprises assessing a behavioral, physical or physiological change.

56. The method of claim 55, wherein assessing a physiological change comprises assessing a change in activity of an NK cell of said mammal.

57. The method of claim 54, wherein said assessing the effect of said test compound on said animal comprises assessing the activity or number of marked NK cells in said mammal in the presence or absence of said compound.

58. The method of claim 57, wherein said marked cells contain a GFP marker protein and wherein the activity or number of marked NK cells is assessed by counting or isolating fluorescent NK cells within said mammal using FACS.

59. The method of claim 54, wherein the test compound is a candidate modulator of NK cell activity, and a detection of an increase or decrease in NK cell activity in the presence of said compound indicates that the test compound is a modulator of NK cell activity.


SEQUENCE LISTING (54 sequences)

SEQ ID NO: 1 - DNA, 25492 bp, Homo sapiens
  misc_feature (8129)..(8131): Start codon ATG
ggggacgggcttaccttccactgtgagagttacctagactgtgatggtcctgtaggcttctgaggcgatcgggatcgggcagtgtcagtcttcagctgctaagccgagaagatctgggaaggagtcagagagccttgggccagagttctagctgctctgggagtggctgccaggtgagttgaacagtccgattttcagtggggtcccgcacagatgggatgcggcttaggaggaatcccaggctgtggacattccttggcccagtggccagatttccagtacttgtagcaagctcctgggggaagaggttctggaggaacccctggcagctgcggttcaggcgtttggagttctcgtgtgctggagatgtggctggggtttgtctcatctggatactggagtggaggcaaggaattgcaactcagaaatatgttgctatttggctgcctctactctattactgtacaccttgaaggcgaggttaattaagtcttgttgtggggtttgagggacagaatttaatttttggagctttatttaatgttgggagcagatttggtaataaaatgtatattgagaataagacggccttttgacttagggtctagggctgtaaagcgtctcagggttgctgccaaatgagccatgaactgggctgtgtttttaaatttgatgaaaaagagcctaaacactatctgatttgggagaggtcagataaagaaaaaggagcattaaccttgactatgcctttagcttcagccacctttttaagaggaaattgctgggcagttgggggagggctagtcatggaatggaactgtaagctggaccgggtgtgaggaggggaggtgataaaaggattatagggtggaggagcggaggctgaggaagaattgggacccagctcggcctggcgaggaggggagatgtcagatgggtctgtagaaaaggaagattagaaagactcagcgatgcttggggttgggactgacgggacaggcgggagggaaagaaggaagatttgggacgagttgcactgggcatagagactagggagggaccgatgtgtaaaagaatgcctggatgtcaggcacctcagaccatttgcccattttacaacaagaattatttagatcttgtaggatggaaaaattgaaagtgccgttttctggctatttggaaccactgtcaagtttgtattggggtcaagcagcattgcagaagaaaataaggcatttaggttttaggtcaggtgtgagttgaagaggttttaggtttttaagaacacaggctaagggagaagaaggaggaatggagggtggaaggttgcccatactgaaggaggcaagcacagagaaaagagagagtagagacatggagggaaggggttcaggggttcttaccttccagaaaagcgggaaaggggtcagggcacagaagtaagggattggggtgcagagacaagaggtcggggtgtggaaataagggatcggggtgcagagataagacgtcagggcacagaaataagggatcgggggattcttgccccctagaaaagcggtacttgccactaagggtgaaggagaaggggttggggggttcttgcccccccagaaaagcagagaaggggtagagacacagagaaggagttgggggttcttgcccccccagaaaagcagtacttgccactaagggtgaaggaccaaggcaggcatccccatgtggtcagacacctctgaaacgtgggtgaataatcagagaggtgtccctgcgtgattaaacaccaagggaaggctgccttcccgagtccatgaccggcgctggagttttgggtccacgaataaagcgcgtctcctgtctctaccagaaaaggaaaggaactgaaattaagagaagggagagattgaagagtggaaaggagaaagtggttgagggatagtgagagaggttggagaagagagtaaaaagaggctgcttactggatttaaaattggtgagatgttccttgggctggttggtctgaggacgagaggtcgtaggtggatctttctcatggagcaaagagcaggaggacaggggattgatctcctaaggaagatcccctgattcgagttatggcaccaaatttcactcacgtccgtgtgaagagaccaccaaacaggatttgtgtgagcaacaaggctgtttatttcacctgggtgcaggcgggctgagtccagaaagagagtcagcaaagggagataggagtgcggccgttttataggatttgggtaggtaaaggaaaattacagtcaaaagggggttgttctctggcgggcaggagtggggttcacaaggtgctcagtaggggagcttttgagccgggatgagccaggagaaggaatttcataagataatgtcatcacttaaggcaagaacaggccattttcatttctttcgtggtggaatgtcatcagttaaggcaggaaccggccatctggatgtgtacatacaggccacagggggatatgatggcttagcttgggctcagaggcctgacagtctggatcacctgacctggtgatccgcacacctcggcctcccaaagtgctgggattacaggcatgacccactgcacctggccttagaaaacttcttaaatattaaaatgtatgttatgtgtattttgccacaatttttgaaaagtaccttctggtgtttagagacagaagatgagtggttgcctagggccgggagagtgaggggatcgtggtgatgggcagctggtcggcatggggttctgaagggcagtgatgacaacattctaaaattagactgtgttgacggttgcaccaactccgtgaataccacaaaatttaaaccattgaattatgcacttttaatgggtaattgtatggcatgtaaattatatctcaataaagttatatttttaaataccaaaaaaaggccgggtgcggtggctcacgcctgtaatcccagcactttgggaggccgagaagggcggatcacgaggtcaggagatggagaccatcctggctaacatggtgaaaccccatctctactttgaaaaaaaaaaaaaaaaaaaaaagattacccggacgtggtggtgggcacctgtagtcctagctactcaggaggctgaggcaggagaatggcataaactcgggaggcagagcttgcagtgagtcgagattgcgccactcaggaggctgaggcaagagaatggcataaaccccggaggcagagcttgcagcgagccgagattgcgccactgcactccagcatgggtgacagagcaagagtccatctcaaaaaaaaaaaaaaaaagattagtaatatcctctgtgtcacttaccacttaagtgattgaatcacgacttgaaattcatcatctcaaacatggcttagagtctgtagaggggggacagtcccaggaatgctggtgtgggcttaaggctgaattaaatagatccagatggctcacacctgtaatcccaataccttgggaggccgaggcaggtgggaggctgaggcaggcggatcactggagctcctggagcgaagaaaggatgctagtggaaaaactggtgaaatcagaataaagtctatagttttattttttaaaggaggctgggcgtggtggctcatgcctctaatcccagcactttgggaggctgaggcaggtggatcagttgagttcaggagttcgaaaccagcctggccaacttgacgaaaccccatctctactagaaatacaaaaattagctgggcgtggttgtgggtgcctctaatcccagctactcaggaagctgaggcaggagaattgcttgaacccaggaggcggaggttgcagtgagctgagatcacaccattgcactccagcctgggctacagagcaagattccatctccaaataagagagacatgacaattaaataaattgtgtaatcttggattaaatcctaaaccaaatatatgtcactggtaaaacaagtggtgaaatttgaataaagtggatagatcagacaatagtgtcatatcagtgctatttcttgaccttgaacattaataacagaatgtccttggttttgggaaatataacctgaagtgattagaggtttagggcatcatatgcaaattagacacactttcttcggggagagagggagagggagagaggctgaatgatgaagcaaatgtggtaaaatgctaactttggggaaatctggatgaagaaattacagattttttttttttttatagacagggtaacactctgtcacccaggctagagtgcagtggcacgatcatggctcactgcagcttctacctccctgggctcagatgaccctctcacctcagcctcccaagtagctgggactataggcgcacagcaccacacctggctaatttttgcatttttttttcccccaggctcgtctcaagcaatccacccacctcggcctcccaaagtgctgggattacaggtgtgagccactgcacctggccagaaattctttaaactatttttgcaagtctggaattatgtcaaaattaaaagctcaaaataataaaagacaatattcttatatttctttggtgaaggtaactatgttatggctgagagggtggctgaggtctgaggatccagcctacataagtctcctccatagagggcatccaagcgctccgtagggggaaggataaagaaaacacccagagttatgacagctgtgtaaggggaaacgccagcaccgagtactgaatcttcagtaaataagaaggaggcgggctgggtgtggtggctcacgcctgtaatcccagcactttgggaggctaaagtgggctgatcacttgaggtcaagagttcgagactagcctggccaacatggggaaaccctgtctctactaaaaatacaaaaattagtcgagtgtggtggcacacgcctgtaatcccagctacttgggaggctagaacaggagaattgcttgaacccaggaggtgaaggttgcagtgagctgagattgcaccactgcaccccagcttgagggacagagtgagattccgtcttaaagagaaaaaaaaaagaattagcacatttgtttgcctcaagaagatacaactagtcttgtacagtagtcacatgtatccaccaggatatattccaaggccccagtggatgctgaaaactacatagtaccttacatgtatatatatatgtatatacatatatacacatatacgtatatgtatacatacatgtatatatgcatgtatgtatatacatatatgcatatatacatacatgtatatatacatgtatgtatatacatatatgtatatgtatgtatacacgcatacatgtatgtatacacgcatatatgtatgtatatacatatatgtatgtatacacgcatacatgtatgtatatacatatatgtatgtatacacgcatacatgtgtgtatacatatatatgcatgtatgcatgtgtgtatatatacatatatgtgtatatatacgcatatacatgtatgtgtatatatgcatgtgtatatatacatgtacggtactatgcagtatatatacacatatatgtatatatgtatacatatatgtataaatgtatatatgtgtatatatataaaaggtatatatgtatatatgtgtgtatatataaaatgcatgaatttcttttttcttactgtagatcttaacaacttctgcatagaatttttttttattaagtggagagttagttacttacttaaaagaaatgtttcttggctgggtgtggtggctcacacctgtaatcccagcactttgagaggccgaggcaggaagattcacttgaggtgaggagttggagaccatcctggccaacgtggtaaaaaccggtctctactaaaagtacaaaaatgagctgggcgtggtgttgggtgtctgtagtcccagctactcaggtggctgaggcaggagaattgcttgaacccacaaggcagaggttgcagtgagctgagatcacaccactgcaccacagcctgggcaacagagcaagactctgtctcaaaaaaaaaaaaaaaagaaagaaaaagaaaaagaaaagaaatgtttcttttcttattaagttctttaaatgaaaagcttttcttttcacttttattttattgaaacattataacactatctttgaagaagttagtgttatcattccattctgatgaaaccaattaacttatccaagcatatgtatactgtacacagagaagccaacgtcaaaacccctatttttatctttttagattcagcagatacatgtgcaggttttttatgagtatattgcatgatgctgaggcttgcattaatgatctagtcaccaaataggtagattttcaagccttgctcccctccttacccaatgtttagcgctctcacttataagtgagaacatgtggtatttggttttcttttcttttttttttttttttttgagatggagtttcactcttgttgcccaggctggagtacaatggcaccatctcggctcactgcaaccttcaccttccaggttcaagcaattctcctgcctcagcctcccgagtagttgggactacaggcatgtgccaccacacccggctaattttgaatttttagtagagacagggtttctgcatgttggtcaggctggtctcgaactcccgacctaaggtgatccacctgcctcagcctcccaaagtgctgggatgacaggcgtgagccaccgtgtctggccagtatttggttttctgtttctgtgttaactcgcttaggataatggcctctagctgcatccatgttgctgcaaaggacataatcttgtgatttttcaaggctgtatagcgttctgtggtgtatacatatcacattgtctttatccagtccacctctgatgggacctgggtggattccatgtcttcactattgtgaatcctgctgcaatgaacatacaagtgcatgtgtctttttggtagaatgatttattttcctttggctatatacccagcgatgggattgctgggctgaatggtaactctgtttgtagttctctgaaatatctccaaaccaaactgctttccacagtggctgaactaatttacacccaccaacagtgtataagtgtcccctttgctccacaatctcaccagcatctgttaatttctggcttttcagtaatggccattctgactggtgtgagatggtattgttgagggataatttaggaatcagagagaccgaggggttgaggaggatttattattattattattatttaggtgcaccggccccagtcagattaacatccaaaaagactgaggctcgaacagagagtccggttaccttttaagcattttgtggggttgggggagatctgtgcagggggaagcatattacagaagcaagaaacaaaggcagttattcaattgagacatgcatcacattattccttacttttcaagaaaaatatgttttacgacttgaggttatcctgtctagtgatcttgcagccgcacggcaagagaaacagggtcttcacaatgcctgggaaagggagagataaggctcactagccacagacagaaaaacaggcagttcatgtttaaaggactccacctctttctcttcctcggggggaactgggttttcttaaatacaactgagtttttgtttacacattctgtaatttcttttaattcctgttccagtatctcactgtgaaactccctatgtttttatacgattctcagggggtttcctctgggcatgattgggcacaacttcccacagtcagctctgggtacgacctccacattgcagaattgagaagttgacccagaaatgcattttgggctgagcagacaattgtcagagttgctggctagaccacagatgtgtcagagggaccacggcctttctgtaagctcatggtcagaggcggaggggagttgtgaacgttctgatgaaagcagtcaacgtgaaagcgctctggtgatgggcgctggtgctcacccaccacttcctgtgtatctatctccctggcccgcccggctcagtccccactgctcagcactaggccggcagaatctgagcgatgtcttccacactccctgccctgctctgcgtcggtgagttctggcgtggaaggggaatgggatcacggtgtgcctgggaggcaacaggtctcattactcccgtcttccagggctgtgtctgagtcagaggatcagcgcccagcagcgtgagtccttccttcaaagcccagggtcactcttccgggttcaggccaagctccttccacccaagcacggctggggagaggggacagggtgctggcttcccaggagagcttggggccagcagctgggtggagcctaaggttggggggagggggctccgctggaactccagcctctgattcccttccagagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacaggtaactgtccggttctctaactggagagtgatctcagtctgcatccgggatgcagcatcatctatgaactcttccaagccccactcagacactgcttgtctcggtaggaggctggaaggaggggtgatccccatcacaatccttgcctacaaggggttgtctgcagaccgtgtctctacgtcctaggagcagatgtgtcctcagtcagtttctccatgacacagattctgagatagatatttgtatgcaggggtatgactgaggaatgtcctcaaaaacaatgcctgtgggctaggcgcagtggcttacactttgcttccctcacccatcacaggtggtgggttttttttttttttatctgttttgagacggagtttcgctcttgtcacccaggctggagtgcagtggtgcaatctccagtcactgcaacctccacctcctgggttcaagtgattctccagcctcagcttcccaagtagctgggatcacaggcacccaccactacgccacattttgtatttttagtagagatggggtttcaccatgttggccagggtggtgtcgaactcctgacctcagatgatccgcccgcctcaccctcccaaagtgctgggattacaggtgtgagccatcacacccagccaggtggtggttttctaaaaaaaaaaaaaaaaattagctttttttttttttaacaatatggttgtttattattattatcaagtattatacatagttacatatacatacataattgtatgtgctatacaattaggtttgtttataccagcaacaccaaaaacacatgagcaatactttgtgctaggaaggctatgatgtcatcaggcaataggaatttttcagtttcattataatcttatgggaccaccatcatatatgtggtacattgttggccaaaatgtcattatgcagctcacaacagtatttcatgtccattcaaatatcttcttttgtgaaatgtctatttaaatcttttgcctatttttaaattgggttgcttatattttgattgattaggaaaagttatttctatattctgtgtcatatacttgtgttgaaatatatatattttttgtctgtgccttttcatttgctcagggtctttggaccttgtttggaggttctggcaggggaacacagctactcatttattcttttttttttaatttttttagtatttattgatcattcttgggtgtttctcggagagggggatttggcagggtcataggacaatagtggagagaaggtcagcagataaacatgtgaacaaaggtctctggctttcctaggcagaggtccctgcggccttccgcagtgtttgtgtccctgggtacttgagattagggagtggtgatgactcttaaggagcatgctgccttcaagcatctgtttaacaaagcacatcttgcaccgcccttaatccatttaaccctgagtggacatagcacatgtttcagagagcacggggttgggggtaaggtcatagattaacagcatcccaaggcagaagaatttgtcttagtacagaacaaaatggagtctcctatgtctacttctttctacacagacacagtaacaatctgatctctctttcttttccccacatttcccctttttctattcgacaaaaccgccatcgtcatcatggcccattctcaatgagctgttgggtacacctcccagacggggtggcggccgggcagaggggctcctcacttcccagacggggcggccgggcagaggcgcccccccacctcccagacggggcagtggccgggcgggggctgccccccaacctcccggacggggcggctggccggggctttttttttttttttttgagacagtctcgctgcagtgcagtggtacaatctcagctcactgcaacctctgcctcagcctcaattctcctgcctcagcctcccaagtagttgagattacaggcatgtgccaccacacccggctaatttttgcatttttagtagagacggggtttcaccatgttgaccaggctggtctcaaactcctgacccaggaggtcgagtcttcagtaagcaaagatagtgccacggcgctccagcctgggaaacagagcaagaccctgtatcatttttaaaaatggttttagacggtaaatcttctattgtgtgtatttgaccaaaataataattaaaaaaaaaaaaaaagctggctgccaggcatggtggcaggcccctgtagtcccagctacttgggagggtgaggcaggagaaacgcttgaacccgggaggcagaggttgcagtgagccaagatcgtgtcactgcactccagcctgggcgacagagagagactccatctctaaagaaagaaaaaaaaaaatagctggctgctcatcactgagtttctggtgtggtggccccaccttctctcatagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggtgaggaaatgctcaattccccacacccttcgccgccatgtcctacctggagccctgagggatccccagagagtgatggggagggtgtccaagggacgtccacttcctgggtgcctggttggtcatgtgaggaagaacaccagaagcaggaaggaggagggagcagagaaaggaatggtaaggcgggtggatcacaaggtcaggagttcgagaccagcctggccaagacggtgaaaccccgtctctactaaaaatacagaaattagccagacgcagtggcggacacctgtagtcccagctactcaggaggctgaggcaggagaatcgcttgaacccgggaggcgggggttgtagtgaaccgagatcataccaccgcactgcaacctgggcgacagagcaagactccatctcaaaaaaaaaaaaaaaaaaaaaaagaatggcaagaccggaggaaaccaaaaacccttacttttttttctttatctccttttccaggcgacattgagaacaccagccttgcacctgaagaccccacctttcctggtgagtaactggtccttctaagctcagacgagcgatcagagcctcccagtgacactaaaaacgtggcattcattcaaaatattcatcgaggccaggcgtggtggctcacgcctgtaatcccagcactttgggaggccgagatggtgcatcatttgaggtcaggagtttgagaccagcctggccaacatggcgaaaccctgtctctactaaaaatacaaaacttaggctgggcatcatggctcacacctgtaatcccaacacttcgggaggccaaggtggttggatcacaaggtcaggaattcgagaccagcctgaccaacatggtgaaaccccatctctactaaaaatacaaaaattagccgggcctggtggtgctcgcctgtaatcccagctactcaggaggctgaggcaggagaattgttgaacctgggatgcagaggttgcagtgagctgagatcgcgccactgcattccactccactgcacgacacagcgagactccatctcacagaaaaacaaaaacaaaactattatatatatatattcatcaagtgcatagtatacacagtgaactacactgtaacagtcagccaggcagatatcttgactctgcagcacttagattctagcaggaggagacacaccatcggtcaacgtcaggatagcacacaggagggaatgatgctatggaaggaaaagacaaagtagaacagacttacagtgattgaaatggcagctagcaatattaaataggtttgtccagatggacctcacagagaaagaaggcatctgagcaaatgcgttcagacttgagttaatcatgtggctgtcaggagaaaggaggctctggagagaatgaaatggcatctgcctgtgccctggggcaggaagataactggggtaatacaataataactatgaggccaggagggttgaaaatgatgtttggaagatgacggtgggatgggcctggggcgcacggctaggattacaggagtgaggcccggcgcggtggctcacgcctgtaatcccagcactttgggaaaccgaggcaggtgggtcatgaggtcaggagatcaagaccatcctggctaacacggtgaaaccctgtctctactaaaaaaaaatacaaaaattatccgggcgtggtggcgggcgcctgtagtcccagctacacaagaggctgaggcaggagaatggcgtgaacccgggagacggagcttgcagtgagctgagatcgcgccactgcactccagcctgagcgacagagtgagactccgtctcaaaaaaaaaaaaaaagaaaaagaaaaagaaaaaaaaatagtgagactttgaatttcactatgtgtgtatgtgtgaggagaaagaggtaatgatgacttaatgaggaaaatgaggcttaaatagaagacgggctgggccgggtggctcccgcatgtaatcccagcactttggaaggcaggggcggctggatcacttgaggtcaggagttcaagaccagcctggccaacacagtgaaaccccatctctactaaaaatacaaacatgagttgggtgtggtggcgcacgccagtaattacagctactcggggctgaagcaagaggattgcttgaactcgggaggcggaggttgcagtgagctgagatcacaccactgtactccagcctcagaggcctgtcatcccagccctttgggaggccgaagcaggcaggtcatctgaggttgggagttcaagaccagcctggccaacatggcaaaaccccgtttctactaaaaatatgaaaaaaattacctgggtatgtggtgtgtgcctgtagtcccagctactccagaggctggaacacagtgagactctatctcaaaaaaaaaaaaaatagaagacatgactggtgcaaagacacatgctcacaagtgctagaatggaattcctcgtcaggttcgtccatctgtggacccttccactttacctgctggatgaagctcctgggacccgcagggtgaggtgggaccttgtaaagctgcagaacgtcatggggtagacccaagggaaggagtgctggggtggaggaggtcaaaaccatcctcttttcttcacttcccttatcatcagcagacacttggggcacctaccttttaaccacagagacgggactccagaaaggtaagtagacagctggggccataggctctgaaggaaggggctgggcatagagtagacctaggaagggaatctaaatgggaacaagagggtgtccttggccaggcgcagtagctcacacctgtaatctcagccctttgggaggccgaggcgggcagatcatctgaggtcgggagttcaagaccagtctggccaacatggcgaaatcccatctctactaaaaatacaaaaaaattagccaggcgtggtggcgtgtgcctgtagtcccagctacttgggaggctgagacaggagaatagcttgaacccaggaagtggaggttgcagtgagccgagatcgtgccattgcactccagcctgggcgacaagactgaggctctgtctcaaaaaaaaaaaaaaaaaaaaaaaaaaaaaaaaaaaaaaagagggtgtccttacatccctgtcagcgatcaccctgttctcctgcctacagaccatgccctctgggatcacactgcccagaatctccttcggatgggcctggcctttctagtcctggtggctctagtgtggttcctggttgaagactggctcagcaggaagaggactagagagcgagccagcagagcttccacttgggaaggcaggagaaggctgaacacacagactctttgaagaatgaccatgagacacagtggccatgggtggatctgaaagctggtgttgagcctgggcggcgtgagctctgtgttggacccacggaggagggagtcactgcagggaaagagggacactggcattccatttgtcagagcatcccggacgatgcagagggtgggagaactacatgctaaatttctttttttttttttttgagacagagttttctcttgttgcccaggctggagtgcaatggcgcgatcttggctcactgcaacctctagctctccatccctcgggttcaagtgattctcctgcctcagcctcctgagtagctgggattacaggcatgtgccaccaccccagctaattttgtatttttagtggagacggggtttctccctgttggctggtctcgaactcctgacctcaagtgatctccccgccttggcctcccaaagggctgggattacaggcataagccgctgcgcccagccactgaatttcttctgtagacaaatcctatggtctcttctaggctctaactatttttgtaccacttactgcaaaccatacttttaaccactctggtcttttctgaaaagatctctccttctttaacaggatggccatggaaatatttttttcctactttggtctttttttctttcctttctctgcaggaagccattcaaaatagttaataaccaatatagaataggtctgtatcaaatggttcaggaggcattgtggcaacaaccagttgtagagaagcagctttataagtgaatcctgccaggcacggtggctcacacctgtaatcccaacactttgggaggctgaggcgggcagatcacctgaggtcaggagttcgagaccagcctggccaacatgatgaaaccccatctctactaaaaatacaaaaactcggccaggcacggtggctcatgcctgtaatcccagcactttgggaggccaaggtgggaggatcacctgaggtcaggagttcgagagcagcctggccaacatggtgaaaccacatctctactaaaaatataaaaattagccaggtatggtggcgtgtgcttgtaatcccagctactcaggaggctgaggcaggagaatagcttgaacccgggaggcggaggctgcagggagccaagatcgcaccactgcactccagcctacgtgacagagcaagattctgtctcaaaaaaaaaaagaaaaaaaaaaaataagtgactcctggctgcatcccaaccataccccaattccttctaaccacagaattattccatcttctcttccttttttttttttttctttttttttgtttgttttgttgggacagaatttcactttttttttttttaatgtaagttttagggtacatgtgcacaacgtgcaggttagttacatatgtatacatgtgccatgttggtgtgctgcacccactaactcgtcatttaacattaggtatatctcctaatgctatcccttcccccgagtttcacttttgtcacccaggctggaatgcagtggtgcaatcttggctcactgccacctccacctccagggttcaaatgattctcctgcctcagcctcctgaatagctgggattataggcatgcaccaccacgcccggctaatttttgtatttttagtagaaatggggtttcacaatgttggccagactggtcttgaactcctgacctcaggtgatccaccagcctcggcctcccaaagtgctggaattacaggtgtgagtcaccgtacccggccaccatctttgcttctttatccacaccttgccttgttcttcagggctctgcagagatatcatttcctccaagagtttccacaactccgacttcacaaagatagcacttttttttttttttttgagacagtctcactctgtagcccaagctggcgtgcagtggcacaatctcagctcactgcaaccttcgcctctggggctcaagcgattctccttcctcagcctcccaagtagctgggactagaggcgcgcgccaccacacccggttaattttttttgcatctttagtagaggtagggtttcatcatgttgccctgggtggtctcaaactcctgagttcaggtgatccccccgccttggcctctcaaagtgctaggattacaggcgtgagccactgcgcccagccaagacaacactttcctcatcccaaagcacctgttaattccctgtaacagcacttgaaccctgattcggcatgcatgtccattttcctgcctctaccgtgaactcgtgtgaattgatctatgtcagatttagtggctgcattcacagctcccgcaactataacggggttctcgggaaatatatatcaaatgagtgaatgtatatacggggctgtggcacagcctgcaacttgagacttctcactaggggtcttgaaatgctgtctggacaccaccatcgctttcctccctgagaacttctacttatcaacccatttatatactcatcgcatgggtcctcacgccctcccattattctggtgcctcatgccggtcaaatttattctctaaatctgatttttccattaaatagcagcctggccaacacggtaaaaccccatctctactaaaaaatacaaaatattagccaggcgcagtggcttgcacccgtaatctcagctactcgggaggctgaggcagcagaatcacttgaacccgggaggcagaggttgtggtaagccgagattgcaccactgcactccagcctggtaacagagcgagactccctctcaaaataaataaactgctgactcgcgtattttttctttaccccaactcattccttacatgtaggcacctgtaatcctagctactcagaaggctgaggcaggagaatcgcttgaacctgggaggcggaggttgcggtgagccaaaatcgtgccactgcactccagcctgggcgacagagcgagactccatctcaaaaaaaaaaaaaaaaaaaccacataggctcagtcttttcagtatctgctttactggttcagtaaaagccaggaaacacaactttgtggtaatctgaatgttattgaactgtattttgttcactttattgtaaatactagtgaacagtgaataaatggttgtatattcctaataagaaaaaaaaaaaaaaagacccaaagtacagcgagctgatgccgatctcatttcgcagaggtccgcctgctctcccctctccaagagtgtaatcctatgcttaataaacttatgccgctttgctatgtgtgtgtatcacacccaattctttgttcgaaacaccaagggcctggaacttcacagctttggctggtaacggggagcaggggtaaagacatttaaaagctgcttgtgttaaccataatcgccatcccatatatcagacccccagaactaactcatcttataactgaatattgtgctttttttttttttttttttttgagacgaagtcctgctctgtcacccaggctggagtgcagtggcgcgatcttgactctgcaacctccgcctcccgggttcaagcgattctcctgcctcagcctcccgagtagctgggactacaagtgcgtgccaccacgcccggctaatttttgtatttttagtagagacggggtttctccatgttggtctcaaactcctggtctcaggtgatccacccgccttggcctcccaaagtgctgggattacagacgtgagccaccacacccagctacttgtgctttttgaccaacatcttcctctcctaccacccccagcccctgataacctccacctactctcacttctaggagatcaactgttctatttttttttttttttttttttttttgagtctcgctctgcacacccaggctggagtgcagtgctgcaatctcggatcactgcaacctccgctttccgggttcaagcgattctcctgcctcagcctccagagtcgctgggattactgagccaccgcgcccagccagaagacccacgctccctaagacataacccacactggtggcctttgttctgacttctcacctgtgctccccacccgctagaaactggcttctctccccacacttcctctgaagctgtctgtgtgaccaacactaatgagcttccttcctggaacatgcagtgacccttttcagcccttctcattattgctcccccacagttgtatttgacacgttgaccacttcctcctcgaaggactcacttctctggctttctcggacacttcttgctactcgttttctgacggttacagtaccaacaggtttgcaggcacctccaccaccagagccaatcccagctactcgggaggctgaggcaggagaatcgttcaaacccgggaggcagaggttgcagtgagtcgagattgcgccactgcactccagcctgagtgacagactgtgactcctcaaaaaaaaacaaaaacaaaaacaaaaaaactacagtcttgctctgtcgcccaggatggaatgcagtggtgccatcttggctcactgcaacctctgcctgctggggtctagcgattctcctgcctcagccccccaaggagctgggactacaggcatgtgccgccacgcctggctaatttttgtatttttagtggagatgggggttttaccatgttagccaggttggtcttgaactcccgacctcatgtgatccgcccaccttggcctcccaaagtgcgaggattacaggcccccgcacccagcctaggatcctgcacctctctagcctagcagttctctgctgggtgattttgctctccactccaggggacatttggcaatgcccatggtaatttttaattgtcatgacttggggaggggttctactggcatctggtaggtagggtccaggggtgctgctcagcttcctacaatgcccagggcagccccagatggcagcagcaccaaggctgagaaacactggctcatgcagaaagcaaccaccttacacccttcagtgcagggacaaaggcagggttacgagtccacggaaactctccagtctcagcctacgtaagacgtggctatttttctttcttattgtttttattcatttatttttcttgagacagagtcttgctctgtcgcccaggctggactgcagtggcgcgatctctgctcactgcaagctccgcctcccgggatcacaccattctcctgggactacaggcgcccgccacctagcccggctaattttttgtatttttagtagagacggggtttcaccatgttagccaggatggtctcgatctgacctcgtgatcctcccgcctcggcctctcaaagtgctgggattacaggtgtaagccaccgcacccggccttattcatttattttttgagatagagtctgagccctttattttatttatttagagaccaagtctcgctctgttacccaggctggagtgcagtgtcgtggcctcagctcactgcaacaacctccgcctcccgggttcaagcgattctcccaccttggcctcccaaagtgctggcattacagacacccactaccatgcctggctaattttttgtacttttagtaagtaaagacagggtttcaccatcttggtcaggatggtctcgaactcctggcctcaagtgatcggcccgcctgggtctcccaaagtgatgagattacaggcgtgagcgaccacactggcctaatgtgtagttttttatctgtggcctcccttctgccctcccccttctgagactctgaagcccattacatcactctgcctttgtgtaccaacagcttagctcccactgagaacatacagagccaggcacggtggcggtggctcacgcctgtaatcccatcactttgggggtgctgaggcaggtgtatcgcctgaggccaggagttcaagaccagtctggccaacatggtgaaaccccatctctactaaaaatagaaaaatacatagctgggtgtggtggcacgtgcctataatcccagctactagggaggctgaggttggagaatcgcttgaacccaggaggcggaggttgcggtgagccaagatcacaccattgcactctagcctgggcaacaagagcaaaactgtcttaaaaaaaaaaaaaaaaagtgagaacatatggattctactcctgttagaataatggcctccagctccatccaaattgctggaaatgacattatttcattccttctaatggctgaatagtattccatggtacatagacaccacgttttctttatccactgtagggaccagccccacagggtcggtgggtctctccctgtgtgcggcgacgagagagtgtagaaataaagacacaagacaaagagacaagagaaaaggcagctgggcccgggggaccactaccaccaatgcgcggagaccggtagtggccccgaatgtctggctgcgctgttatttattggatacaaggcagaaggggcagggtaaagagtgtgagtcacctccaatgataggtaaggtcacgtgggtcacgtgtccactggacagggggcccttccctgcctggcagccgaggcagagagggagaggagacagagagaaagacagcttacgccattatttctgcatatcagggactattagtactttccctaatttactactgctatctagaaggcagagccaggtgtacaggatggaacatgaaggcggactaggagcgtgaccaccgaagcacagcatcacagggagacggttaggcctccggataactgcgggcgagcctgactgatgtcaggccctccacaagaggtggaggagcagagtcttctctaaactcccccggggaaagggagacccccccccccacccgctgcccctttcccggtctgctaagtagcgggtgttgttaattgacaccttttgctaccgctggaccatgatccgcttggtgacgggtgtcttcccagacgctggcgtcaccgctagaccaaggagccctctggtggccctgtccgggcataacagaaggctcgcactcttgtcttctggtcacacctcactatgtcccctcagctcctatctctgtatggcctggtttttcctaggctatgattatagagtgaggattattataatattggaataaaaagtaattgctaccggctaatgattaatgatactcatatataatcatatctaagatctatatctggtataacaattcttgttttatattttattatactggaacagctcgtgtcctctgtctcttgcctcggtgcctgggtgccttgccgcccacaatccactcattattcaatgggcacttcggttggttccacatctttgcaattgtgaatggctgagccagccattcttaactgggggtgattttgtccccatgggggtatctggccacatcccgagaggttttttggttgtcacgagttgcagtgggggcaggctcaggctcatccaagtccaggggtgctgctatacatcacgtgatacacaggacagtccttgctacggactgaattggtcccaccaaacgtcatgtacaagccctaccccagatgtgactctatttggacacagggcttttcagaggtaattaaggctggtcaggcgccgtaatcacagcactttaggagttctgtgtttattactggtaagtgggtaagagcccagtgtggcagctcacgcgtgtaatcccagcactttgggaagcgaaggcaaggggataactggaggccagcagttcaagacaagcctggtcaatacagcaagactccatctctataaaatattttaaaattagccaagcatgtttggcatgcacctgtaatcccagctcaggaggctcaggtgggaggattccttgagtttaaggctgcagtgagctaagatcgcaccattgcactccaacccggctgtgggcaacacagcaccaccaccatcttggctgggcacggtggctcacgcctgtcatgccagcactttgggaggccgaggcgggtggctcacctgaggtcaggagtttgagactagcctggccaacatggtgaaatcacgccactgcactccagcctaggcaaccaagtgagactctgtccgccccaccaccccaccaaaaaaaagactactatcttaaacaaaatcaaaatttttaagtagataaaatatttaggggaaaaaaacttcaattaaatatgcagcagagtccgacccagatgttttcactcccagcctctacctactatctttgtgtctttatttttagcaaattctacacgggaacttcatgtgcatgtagaaccctaaatgttgactcagccctacctctcatcacctgaccacttcctttattcacgctgtctctaccacccttcccatcggtgtgagctgtatcccgctaaacactgttaccacccacagcctgcattactaccagctgactgtagccttaaacaccacagtgatctcgagcatttgagaagacttatcttgacaagggctcacgaaagacagcaatgctcaacagcaagataaatgagggccttcatgggatcattcagtgctgaagccactcaacctccaggtttgggttagtaaaaagaactttgtgtcgacgcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcagtcgac

SEQ ID NO: 2 - DNA, 90 bp, Homo sapiens
  misc_feature (1)..(90): MBL46-8kb recombination primer (See Example I)
tcagaagcctacaggaccatcacagtctaggtaactctcacagtggaaggtaagcccgtccccgtcgactgaagacgaaagggcctcgtc

SEQ ID NO: 3 - DNA, 92 bp, Homo sapiens
  misc_feature (1)..(92): MBR46+8.6KB recombination primer (See Example I)
atgggatcattcagtgctgaagccactcaacctccaggtttgggttagtaaaaagaactttgtgtcgacgcgctagcggagtgtatactggc

SEQ ID NO: 4 - DNA, 22 bp, Homo sapiens
  misc_feature (1)..(22): ScreenFwdpACYC177 primer  (See Example IV)
aagtgccacctgacgtctaaga

SEQ ID NO: 5 - DNA, 22 bp, Homo sapiens
  misc_feature (1)..(22): ScreenRvsNKp46-8kb primer (See Example IV)
ccatctgtgcgggaccccactg

SEQ ID NO: 6 - DNA, 25 bp, Homo sapiens
  misc_feature (1)..(25): MBhNKp46Ex5 primer from exon 5 of human NKp46 (See Example V)
tgcaaggctggtgttctcaatgtcg

SEQ ID NO: 7 - DNA, 25 bp, Homo sapiens
  misc_feature (1)..(25): MBhNKp46Ex4 primer from exon 4 of the human NKp46 sequence (See Example V)
acccaccctctcggttcatcctgga

SEQ ID NO: 8 - DNA, 1554 bp, Homo sapiens
  misc_feature (1)..(1554): Accession no X97229
gcaccatgtccatgtcacccacggtcatcatcctggcatgtcttgggttcttcttggaccagagtgtgtgggcacacgtgggtggtcaggacaagcccttctgctctgcctggcccagcgctgtggtgcctcaaggaggacacgtgactcttcggtgtcactatcgtcgtgggtttaacatcttcacgctgtacaagaaagatggggtccctgtccctgagctctacaacagaatattctggaacagtttcctcattagccctgtgaccccagcacacgcagggacctacagatgtcgaggttttcacccgcactcccccactgagtggtcggcacccagcaaccccctggtgatcatggtcacaggtctatatgagaaaccttcgcttacagcccggccgggccccacggttcgcgcaggagagaacgtgaccttgtcctgcagctcccagagctcctttgacatctaccatctatccagggaaggggaagcccatgaacttaggctccctgcagtgcccagcatcaatggaacattccaggccgacttccctctgggtcctgccacccacggagagacctacagatgcttcggctctttccatggatctccctacgagtggtcagacccgagtgacccactgcctgtttctgtcacaggaaacccttctagtagttggccttcacccactgaaccaagcttcaaaactggtatcgccagacacctgcatgctgtgattaggtactcagtggccatcatcctctttaccatccttcccttctttctccttcatcgctggtgctccaaaaaaaaaaatgctgctgtaatgaaccaagagcctgcgggacacagaacagtgaacagggaggactctgatgaacaagaccctcaggaggtgacatacgcacagttggatcactgcattttcacacagagaaaaatcactggcccttctcagaggagcaagagaccctcaacagataccagcgtgtgtatagaacttccaaatgctgagcccagagcgttgtctcctgcccatgagcaccacagtcaggccttgatgggatcttctagggagacaacagccctgtctcaaacccagcttgccagctctaatgtaccagcagctggaatctgaaggcgtgagtctccatcttagagcatcactcttcctcacaccacaaatctggtgcctgtctcttgcttaccaatgtctaaggtccccactgcctgctgcagagaaaacacactcctttgcttatcccacaattctctatttcacttgacccctggcccacctctccaacctaactggcttacttcctagtctacttgaggctgcaatcacactgaggaactcacaattccaaacatacaagaggctctctcttaacacggcacttagacacgtgctgttccaccttccctcgtgctgttccacctttcctcagactatttttcagccttctggcatcagcaaaccttataaaatttttttgatttcagtgtagttctctcctcttcaaataaacatgtctgccttcattcttaaa

SEQ ID NO: 9 - DNA, 1128 bp, Homo sapiens
  misc_feature: Accession no AF217485, KIR3DS1 gene
atgtcgctcatggtcatcagcatggcgtgtgttgggttcttcttgctgcagggggcctggacacatgagggtggtcaggacaagcccttgctgtctgcctggcccagcgctgtggtgcctcgaggaggacatgtgactcttctgtgtcgctctcgtcttgggtttaccatcttcagtctgtacaaagaagatggggtgcctgtccctgagctctacaacaaaatattctggaagagcatcctcatgggccctgtgacccctgcacacgcagggacctacagatgtcggggttcacacccacgctcccccattgagtggtcagcacccagcaaccccctggtgatcgtggtcacaggtctatttgggaaaccttcactctcagcccagccgggccccacggttcgcacaggagagaacgtgaccttgtcctgcagctccaggagctcatttgacatgtaccatctatccagggaggggagggcccatgaacctaggctccctgcagtgcccagcgtcaatggaacattccaggctgactttcctctgggccctgccacccacggagggacctacacatgcttcggctctctccatgactcaccctatgagtggtcagacccgagtgacccactgcttgtttctgtcacaggaaactcttcaagtagttcatcttcacccactgaaccaagctccaaaactggtatccgcagacacctgcacattctgattgggacctcagtggctatcatcctcttcatcatcctcttcttctttctccttcattgctgctgctccaacaaaaagaatgctgctgtaatggaccaagagcctgccggggacagaacagtgaacagggaggactctgatgatcaagaccctcaggaggtgacatatgcacagttggatcactgcgttttcacacagacaaaaatcacttccccttctcagaggcccaagacacctccaacagataccaccatgtacatggaacttccaaatgctaagccaagatcattgtctcctgcccataagcaccacagtcaggccttgaggggatcttctagggagacaacagccctgtctcaaaaccgggttgctagctcccatgtaccagcagctggaatctga

SEQ ID NO: 10 - DNA, 1572 bp, Homo sapiens
  misc_feature: Accession no NM_014219, KIR2DL2 mRNA
gcacagacagcaccatgtcgctcatggtcgtcagcatggcgtgtgttgggttcttcttgctgcagggggcctggccacatgagggagtccacagaaaaccttccctcctggcccacccaggtcgcctggtgaaatcagaagagacagtcatcctgcaatgttggtcagatgtcaggtttgagcacttccttctgcacagagaagggaagtttaaggacactttgcacctcattggagagcaccatgatggggtctccaaagccaacttctccatcggtcccatgatgcaagaccttgcagggacctacagatgctacggttctgttactcactccccctatcagttgtcagctcccagtgaccctctggacatcgtcatcacaggtctatatgagaaaccttctctctcagcccagccgggccccacggttctggcaggagagagcgtgaccttgtcctgcagctcccggagctcctatgacatgtaccatctatccagggagggggaggcccatgaatgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcataggaaacccttcaaatagttggccttcacccactgaaccaagctctaaaaccggtaacccccgacacctgcacattctgattgggacctcagtggtcatcatcctcttcatcctcctcttctttctccttcatcgctggtgctccaacaaaaaaaatgctgcggtaatggaccaagagtctgcagggaacagaacagcgaatagcgaggactctgatgaacaagaccctcaggaggtgacatacacacagttgaatcactgcgttttcacacagagaaaaatcactcgcccttctcagaggcccaagacacccccaacagatatcatcgtgtacgcggaacttccaaatgctgagtccagatccaaagttgtctcctgcccatgagcaccacagtcaggccttgagggcgtcttctagggagacaacagccctgtctcaaaaccgggttgccagctcccatgtaccagcagctggaatctgaaggcatgagtctgcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccaaacatacaagaggctccctcttaacgcagcacttagacacgtgttgttccaccttccctcatgctgttccacctcccctcagactagctttcagtcttctgtcagcagtaaaacttatatattttttaaaataacttcaatgtagttttccatccttcaaataaacatgtctgcccccatg

SEQ ID NO: 11 - DNA, 952 bp, Homo sapiens
  misc_feature: Accession no X89892, Eb6 ActI mRNA
ctgtctgcaccggcagcaccatgtcgctcacggtcgtcagcatggcgtgtgttgggttcttcttgctgcagggggcctggccacatgagggagtccacagaaaaccttccctcctggcccacccaggtcgcctggtgaaatcagaagagacagtcatcctgcaatgttggtcagatgtcatgtttgaacacttccttctgcacagagaggggatgtttaacgacactttgcgcctcattggagaacaccatgatggggtctccaaggccaacttctccatcagtcgcatgaggcaagacctggcagggacctacagatgctacggttctgttactcactccccctatcagttgtcagctcccagtgaccctctggacatcgtgatcataggtctatatgagaaaccttctctctcagcccagccgggccccacggttctggcaggagagaatgtgaccttgtcctgcagctcccggagctcctatgacatgtaccatctatccagggaaggggaggcccatgaacgtaggctccctgcagggaccaaggtcaacggaacattccaggccaactttcctctgggccctgccacccatggagggacctacagatgcttcggctctttccgtgactctccatacgagtggtcaaagtcaagtgacccactgcttgtttctgtcacaggaaacccttcaaatagttggccttcacccactgaaccaagctccgaaaccggtaaccccagacacctacatgttctgattgggacctcagtggtcaaaatccctttcaccatcctcctcttctttctccttcatcgctggtgctccgacaaaaaaaatgctgctgtaatggaccaagagcctgcagggaacagaacagtgaacagcgaggattctgatgaacaagaccatcaggaggtgtcatacgcataattggatcactgtgtttt

SEQ ID NO: 12 - DNA, 1129 bp, Homo sapiens
  misc_feature: Accession no L76667NKAT5-delta-Ig1/Ig2 mRNA
cagacagcaccatgtcgctcatggtcgtcagcatggcgtgtgttgggttcttcttgctgcagggggcctggccacatgagggagtccacagaaaaccttccctcctggcccacccaggtcccctggtgaaatcagaagagacagtcatcctgcaatgttggtcagatgtcaggtttgagcacttccttctgcacagagaggggaagtataaggacactttgcacctcattggagagcaccatgatggggtctccaaggccaacttctccatcggtcccatgatgcaagaccttgcagggacctacagatgcttcggctctttccgtgactctccctatgagtggtcaaactcgagtgacccactgcttgtttctgtcacaggaaacccttcaaatagttggccttcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataattggatcactgtgttttcacacagagagaaatcactcgcccttctgagaggcccaagacacccccaacagataccagcatgtacatagaacttccaaatgctgagcccagatccaaagttgtcttctgtccacgagcaccacagtcaggccttgaggggatcttctagggagacaacagccctgtctcaaaaccgggttgccagctcccatgtaccagcagctggaatctgaaggcatcagtcttcatcttagggcatcgctcttcctcacaccacgaatctgaacatgcctctctcttgcttacaaatgtctaaggtccccactgcctgctggagagaaaacacactcctttgcttagcccacaattctccatttcacttgacccctgcccacctctccaacctaactagcttacttcctagtctacctgaggctgcaatcacactgaggaactcacaattccaaacatacaagaggctccctcttaacacagcac

SEQ ID NO: 13 - DNA, 1113 bp, Homo sapiens
  misc_feature: Accession no L76670, nkat7 mRNA
ccggcagcaccatgtcgctcatggtcatcagcatggcatgtgttgggttcttctggctgcagggggcctggccacatgagggattccgcagaaaaccttccctcctggcccacccaggtcgcctggtgaaatcagaagagacagtcatcctgcaatgttggtcagatgtcatgtttgagcacttccttctgcacagagaggggacgtttaacgacactttgcgcctcattggagagcacattgatggggtctccaaggccaacttctccatcggtcgcatgaggcaagacctggcagggacctacagatgctacggttctgttcctcactccccctatcagttttcagctcccagtgaccctctggacatcgtgatcacaggtctatatgagaaaccttctctctcagcccagccgggccccacggttctggcaggagagagcgtgaccttgtcctgcagctcctggagctcctatgacatgtaccatctatccacggagggggaggcccatgaacgtaggttctctgcagggcccaaggtcaacggaacattccaggccgactttcctctgggccctgccacccaaggaggaacctacagatgcttcggctctttccatgactctccctacgagtggtcaaagtcaagtgacccactgcttgtttctgtcacaggaaacccttcaaatagttggccttcacccactgaaccaagctccaaaaccggtaaccccagacacctacacgttctgattgggacctcagtggtcaaactccctttcaccatcctcctcttctttctccttcatcgctggtgctccgacaaaaaaaatgcatctgtaatggaccaagggcctgcggggaacagaacagtgaacagggaggattctgacgaacaggaccatcaggaggtgtcatacgcataattggatcactgtgttttcacacagagaaaaatcactcccccttctcagaggcccaagacacccccaacagatagcagcatgtacatagaacttccaaatgctgagtccagatccaaagctgtcttctgtccacgagcaccacagtcaggccttgaggggatcttctagggagacaacagccctgtct

SEQ ID NO: 14 - DNA, 1088 bp, Homo sapiens
  misc_feature (1)..(1088): Accession no AF022048, KIR2DL3 mRNA
atgtcgctcatggtcgtcagcatggcgtgtgttgggttcttcttgctgcagggggcctggccacatgagggagtccacagaaaaccttccctccgggcccacccaggtcccctggtgaaatcagaagagacagtcatcctgcaatgttggtcagatgtcaggtttgagcacttccttctgcacagagaagggaagtttaaggacactttgcgcctcattggagagcaccatgatggggtctccaaggccaacttctccatcggtcccatgatgcaagaccttgcagggacctacagatgctacggttctgttactcactccccctatcagttgtcagctcccagtgaccctctggacatcgtcatcacaggtctatatgagaaaccttctctctcagcccagccgggccccacggttctggcaggagagagcgtgaccttgtcctgcagctcccggagctcctatgacatgtaccatctatccagggagggggaggcccatgaacgtaggttctctgcagggcccaaggtcaacggaacattccaggccgactttcctctgggccctgccacccacggagggacctacagatgcttcggctctttccgtgactctccatacgagtggtcaaactcgagtgacccactgcttgtttctgtcacaggaaacccttcaaatagttggccttcacccactgaaccaagctccgaaaccggtaaccccagacacctgcatgttctgattgggacctcagtggtcatcatcctcttcatcctcctcctcttctttctccttcatcgctggtgctgcaacaaaaaaaatgctgttgtaatggaccaagagcctgcagggaacagaacagtgaacagggaggactctgatgaacaagaccctcaggaggtgacatatgcacagttgaatcactgcgttttcacacagagaaaaatcactcacccttctcagaggcccaagacacccccaacagatatcatcgtgtacacggaacttccaaatgctgagccctgatccaaagttgtctcctgcccatgagcaccacagtcaggccttgaggggatcttctagggaga

SEQ ID NO: 15 - DNA, 1134 bp, Homo sapiens
  misc_feature: Accession no L76671, nkat8 mRNA
ccggcagcaccatgtcgctcatggtcatcatcatggcgtgtgttgggttcttcttgctgcagggggcctggccacaggagggagtccacagaaaaccttccttcctggccctcccaggtcacctggtgaaatcagaagagacagtcatcctgcaatgttggtcggatgtcatgtttgagcacttccttctgcacagagaggggaagtttaacaacactttgcacctcattggagagcaccatgatggggtttccaaggccaacttctccattggtcccatgatgcctgtccttgcaggaacctacagatgctacggttctgttcctcactccccctatcagttgtcagctcccagtgaccctctggacatggtgatcataggtctatatgagaaaccttctctctcagcccagccgggccccacggttcaggcaggagagaatgtgaccttgtcctgcagctcccggagctcctatgacatgtaccatctatccagggagggggaggcccatgaacgtaggctccctgcagtgcgcagcatcaacggaacattccaggccgactttcctctgggccctgccacccacggagggacctacagatgcttcggctctttccgtgacgctccctacgagtggtcaaactcgagtgatccactgcttgtttccgtcacaggaaacccttcaaatagttggccttcacccactgaaccaagctccaaaaccggtaaccccagacacctacatgttctgattgggacctcagtggtcaaaatccctttcaccatcctcctcttctttctccttcatcgctggtgctccgacaaaaaaaatgctgctgtaatggaccaagagcctgcagggaacagaacagtgaacagcgaggattctgatgaacaagaccatcaggaggtgtcatacgcataattggatcactgtgttttcacacagagaaaaatcactcgcccttctgagaggcccaagacacccccaacagataccagcatgtacatagaacttccaaatgctgagcccagatccaaagttgtcttctgtccacgagcaccacagtcaggccttgaggggatcttctagggagacaacagccctgtctcaaaaccgggttgccagctcc

SEQ ID NO: 16 - DNA, 1457 bp, Homo sapiens
  misc_feature: Accession L7662, nkat9 mRNA
ccggcagcaccatgttgctcatggtcatcagcatggcgtgtgttgcgttcttcttgctgcagggggcctggccacatgagggattccgcagaaaaccttccctcctggcccacccaggtcccctggtgaaatcagaagagacagtcatcctgcaatgttggtcagatgtcatgtttgagcacttccttctgcacagagaggggacgtttaaccacactttgcgcctcattggagagcacattgatggggtctccaagggcaacttctccatcggtcgcatgacacaagacctggcagggacctacagatgctacggttctgttactcactccccctatcagttgtcagcgcccagtgaccctctggacatcgtgatcacaggtctatatgagaaaccttctctcccagcccagccgggccccacggttctggcaggagagagcgtgaccttgtcctgcagctcccggagctcctatgacatgtaccatctatccagggaaggggaggcccatgaacgtaggctccctgcagggcccaaggtcaacagaacattccaggccgactctcctctggaccctgccacccacggaggggcctacagatgcttcggctctttccgtgactctccatacgagtggtcaaagtcaagtgacccactgcttgtttctgtcacaggaaactcttcaaatagttggccttcacccactgaaccaagctccgaaaccggtaaccccagacacctacacgttctgattgggacctcagtggtcaaactccctttcaccatcctcctcttctttctccttcatcgctggtgctccaacaaaaaaaatgcatctgtaatggaccaagggcctgcggggaacagaacagtgaacagggaggattctgatgaacaggaccatcaggaggtgtcatacgcataattggatcactgtgttttcacacagagaaaaatcactcccccttctcagaggcccaagacacccccaacagataccagcatgtacatagaacttccaaatgctgagtccagatccaaagctgtcttctgtccacgagcaccacagtcaggccttgaggggatcttctagggagacaacagccctgtctcaaaaccgggttgccagctcccatgtacctgcggctggaatctgaaggcatcagtcttcatcttaggggatcgctcttcctcaaaccacgaatctgaacatgcctctctcctgcttacaaatgtctaaggtccccactgcctgctggagagaaaacacactcctttgcttagcccacaattctccatttcacttgacccctgcccacctctccaacctaactggcttacttcctggtctacttgaggctgcaatcacactgaggaactcacaattccaaacatacaagaggctccctcttaacacagcacttagacacgtgctgttccaccttccctcatgctg

SEQ ID NO: 17 - DNA, 300 bp, Homo sapiens
  misc_feature (1)..(300): Accession no AF204908, KIR15
tcaggtgaagggagctgtgacaaggaagagcctccgtaaggaaaatgcctcttctccttccaggtctatatgagaaaccttctctctcagcccagccgggccccacggttctggcaggagagagcgtgaccttgtcctgcagctcccggagctcctatgacatgtaccatctatccacggagggggaggcccatgaacgtaggttctctgcagggaccaaggtcaacggaacattccaggctgactttcctctgggccctgccacccacggaggaacctacagatgcttcggctctttcc

SEQ ID NO: 18 - DNA, 1857 bp, Homo sapiens
  misc_feature (1)..(1857): Accession no L41269, nkat3 mRNA
tgtctgcaccggcagcaccatgtcgctcatggtcgtcagcatggcgtgtgttgggttgttcttggtccagagggccggtccacacatgggtggtcaggacaaacccttcctgtctgcctggcccagcgctgtggtgcctcgaggaggacacgtgactcttcggtgtcactatcgtcataggtttaacaatttcatgctatacaaagaagacagaatccacattcccatcttccatggcagaatattccaggagagcttcaacatgagccctgtgaccacagcacatgcagggaactacacatgtcggggttcacacccacactcccccactgggtggtcggcacccagcaaccccgtggtgatcatggtcacaggaaaccacagaaaaccttccctcctggcccacccaggtcccctggtgaaatcaggagagagagtcatcctgcaatgttggtcagatatcatgtttgagcacttctttctgcacaaagaggggatctctaaggacccctcacgcctcgttggacagatccatgatggggtctccaaggccaatttctccatcggtcccatgatgcttgcccttgcagggacctacagatgctacggttctgttactcacaccccctatcagttgtcagctcccagtgatcccctggacatcgtggtcacaggtccatatgagaaaccttctctctcagcccagccgggccccaaggttcaggcaggagagagcgtgaccttgtcctgtagctcccggagctcctatgacatgtaccatctatccagggaggggggagcccatgaacgtaggctccctgcagtgcgcaaggtcaacagaacattccaggcagatttccctctgggccctgccacccacggagggacctacagatgcttcggctctttccgtcactctccctacgagtggtcagacccgagtgacccactgcttgtttctgtcacaggaaacccttcaagtagttggccttcacccacagaaccaagctccaaatctggtaaccccagacacctgcacattctgattgggacctcagtggtcatcatcctcttcatcctcctcctcttctttctccttcatctctggtgctccaacaaaaaaaatgctgctgtaatggaccaagagcctgcagggaacagaacagccaacagcgaggactctgatgaacaagaccctgaggaggtgacatacgcacagttggatcactgcgttttcacacagagaaaaatcactcgcccttctcagaggcccaagacaccccctacagataccatcttgtacacggaacttccaaatgctaagcccagatccaaagttgtctcctgcccatgagcaccacagtcaggccttgaggacgtcttctagggagacaacagccctgtctcaaaaccgagttgccagctcccatgtaccagcagctggaatctgaaggcgtgagtcttcatcttagggcatcgctcctcctcacgccacaaatctggtgcctctctcttgcttacaaatgtctaggtccccactgcctgctggaaagaaaacacactcctttgcttagcccacagttctccatttcacttgacccctgcccacctctccaacctaactggcttacttcctagtctacttgaggctgcaatcacactgaggaactcacaattccaaacatacaagaggctccctcttgacgtggcacttacccacgtgctgttccaccttccctcatgctgtttcacctttcttcggactattttccagccttctgtcagcagtgaaacttataaaattttttgtgatttcaatgtagctgtctcctcttcaaataaacatgtctgccctca

SEQ ID NO: 19 - DNA, 1863 bp, Homo sapiens
  misc_feature (1)..(1863): Accession no L41270, nkat4 mRNA
tgtctgcaccggcagcaccatgtcgctcacggtcgtcagcatggcgtgcgttgggttcttcttgctgcagggggcctggccactcatgggtggtcaggacaaacccttcctgtctgcccggcccagcactgtggtgcctcgaggaggacacgtggctcttcagtgtcactatcgtcgtgggtttaacaatttcatgctgtacaaagaagacagaagccacgttcccatcttccacggcagaatattccaggagagcttcatcatgggccctgtgaccccagcacatgcagggacctacagatgtcggggttcacgcccacactccctcactgggtggtcggcacccagcaaccccctggtgatcatggtcacaggaaaccacagaaaaccttccctcctggcccacccagggcccctgctgaaatcaggagagacagtcatcctgcaatgttggtcagatgtcatgtttgagcacttctttctgcacagagaggggatctctgaggacccctcacgcctcgttggacagatccatgatggggtctccaaggccaacttctccatcggtcccttgatgcctgtccttgcaggaacctacagatgttatggttctgttcctcactccccctatcagttgtcagctcccagtgaccccctggacatcgtgatcacaggtctatatgagaaaccttctctctcagcccagccgggccccacggttcaggcaggagagaacgtgaccttgtcctgtagctcctggagctcctatgacatctaccatctgtccagggaaggggaggcccatgaacgtaggctccgtgcagtgcccaaggtcaacagaacattccaggcagactttcctctgggccctgccacccacggagggacctacagatgcttcggctctttccgtgccctgccctgcgtgtggtcaaactcaagtgacccactgcttgtttctgtcacaggaaacccttcaagtagttggccttcacccacagaaccaagctccaaatctggtatctgcagacacctgcatgttctgattgggacctcagtggtcatcttcctcttcatcctcctcctcttctttctcctttatcgctggtgctccaacaaaaagaatgctgctgtaatggaccaagagcctgcgggggacagaacagtgaataggcaggactctgatgaacaagaccctcaggaggtgacgtacgcacagttggatcactgcgttttcatacagagaaaaatcagtcgcccttctcagaggcccaagacacccctaacagataccagcgtgtacacggaacttccaaatgctgagcccagatccaaagttgtctcctgcccacgagcaccacagtcaggtcttgagggggttttctagggagacaacagccctgtctcaaaaccaggttgccagatccaatgaaccagcagctggaatctgaaggcatcagtctgcatcttaggggatcgctcttcctcacaccacgaatctgaacatgcctctctcttgcttacaaatgcctaaggtcgccactgcctgctgcagagaaaacacactcctttgcttagcccacaagtatctatttcacttgacccctgcccacctctccaacctaactggcttacttcctagtcctacttgaggctgcaatcacactgaggaactcacaattccaaacatacaagaggctccctcttaacacggcacttacacacttgctgttccaccttccctcatgctgttccacctcccctcagactatctttcagccttctgtcatcagtaaaatttataaattttttttataacttcagtgtagctctctcctcttcaaataaacatgtctgccctca

SEQ ID NO: 20 - DNA, 1695 bp, Homo sapiens
  misc_feature (1)..(1695): Accession no AF352324, KIR3DL7 mRNA
gtctgcaccggcagcaccatgtcgctcatggtcgtcagcatggcgtgtgttgggttcttcttgctggaggggccctggccacatgtgggtggtcaggacaagcccttcctctctgcctggcccggcactgtggtgtctgaaggacaacatgtgactcttcagtgtcgctctcatcttgggtttaacgaattcagtctgtccaaagaagacgggatgcctgtccctgagctctacaacagaatattccggaacagctttctcatgggccctgtgaccccagcacatgcagggacctacagatgttgcagttcacacccacactcccccactgggtggtcggcacccagcaaccctgtggtgatcatggtcacgggagtccacagaaaaccttccctcctggcccacccaggtcccctggtgaaatcgggagagacggtcatcctgcaatgttggtcagatgtcaggtttgagcgcttccttctgcacagagaggggatcactgaggaccccttgcgcctcgttggacagctccacgatgcgggttcccaggtcaactattccatgggtcccatgacacctgcccttgcagggacctacagatgctttggttctgtcactcacttaccctatgagttgtcggctcccagtgaccctctggacatcgtggtcgtaggtctatatgggaaaccttctctctcagcccagccgggccccacggttcaggcaggagagaatgtgaccttgtcctgcagctcccggagcttgtttgacatttaccatctatccagggaggcagaggccggtgaacttaggctcactgcggtgctgagggtcaatggaacattccaggccaacttccctctgggccctgtgacccacggagggaactacagatgcttcggctctttccgtgccctgccccacgcgtggtcagacccgagtgacccactgcccgtttctgtcacaggtaactccagatacctgcacgctctgattgggacctcagtggtcatcatcccctttgctatcctcctcttctttctccttcatcgctggtgtgccaacaaaaagaatgctgttgtaatggaccaagagcctgcagggaacagaacagtgaacagggaggactctgatgaacaagaccctcaggaggtgacatacgcacagttgaatcactgcgttttcacacagagaaaaatcactcgcccttctcagaggcccaagacacccccaacagataccagcgtgtaacacggaacttccaaatgctgagcgcagatccaaagttgtcttctgtccaccagcaccacagtcaggccttgatgggatcttctagggagacaatagccctgtctcaaaaccgggttgccagctcccatgtaccagcagctggaatctgaaggcgtgagtctgcatcttagggcatcgctcttcctcacaccacgagtctgaacatgcctctctcttgcttacaaatgtctaaggtccccactgcctgctggagagaaaacacactcctttgcttagcccacaattctccatttcacttgacccctgcccacctctccaacctaactggcttacttcctagtctacttgaggctgcgatcacactgaggaactcacaattccaaacatataagaggctccctcttaacacggcacttagatacatgctattccacctttcctcag

SEQ ID NO: 21 - DNA, 1740 bp, Homo sapiens
  misc_feature (1)..(1740): Accession no L76661, nkat10 mRNA
ccggcagcaccatgtcgctcatggtcgtcagcatggcgtgtgttgggttgttcttggtccagagggccggtccacacatgggtggtcaggacaagcccttcctgtctgcctggcccagcgctgtggtgcctcgcggaggacacgtgactcttcggtgtcactatcgtcataggtttaacaatttcatgctatacaaagaagacagaatccacgttcccatcttccatggcagaatattccaggagggcttcaacatgagccctgtgaccacagcacatgcagggaactacacatgtcggggttcacacccacactcccccactgggtggtcggcacccagcaaccccatggtgatcatggtcacaggaaaccacagaaaaccttccctcctggcccacccaggtcccctggtgaaatcaggagagagagtcatcctgcaatgttggtcagatatcatgtttgagcacttctttctgcacaaagagtggatctctaaggacccctcacgcctcgttggacagatccatgatggggtctccaaggccaatttctccatcggttccatgatgcgtgcccttgcagggacctacagatgctacggttctgttactcacaccccctatcagttgtcagctcccagtgatcccctggacatcgtggtcacaggtctatatgagaaaccttctctctcagcccagccgggccccaaggttcaggcaggagagagcgtgaccttgtcctgtagctcccggagctcctatgacatgtaccatctatccagggaggggggagcccatgaacgtaggctccctgcagtgcgcaaggtcaacagaacattccaggcagatttccctctgggccctgccacccacggagggacctacagatgcttcggctctttccgtcactctccctacgagtggtcagacccgagtgacccactgcttgtttctgtcacaggaaacccttcaagtagttggccttcacccacagaaccaagctccaaatctggtaacctcagacacctgcacattctgattgggacctcagtggtcaaaatccctttcaccatcctcctcttctttctccttcatcgctggtgctccaacaaaaaaaatgctgctgtaatggaccaagagcctgcagggaacagaagtgaacagcgaggattctgatgaacaagaccatcaggaggtgacatacgcataattggaacactgtgttttcacacagagaaaaatcactcgcccttctcagaggcccaagacacccccaacagataccagcatgtacatagaacttccaaatgctgagcccagatccaaagttgtcttctgtccacgagcaccacagtcaggccttgaggggatcttctagggagacaacagccctgtctcaaaactgggttgccagttcccatgtaccagcagctggaatctgaaggcatcagtcttcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccacacatacaagaggctccgtcttaacgcagcacttagacacgtgctgttccaccttccctcatgctg

SEQ ID NO: 22 - DNA, 1332 bp, Homo sapiens
  misc_feature (1)..(1332): Accession no AF204919, KIR48
ggggttgatactcctcaaaccagcaccagaagccagcctatggaagctggcaccatggagaaggcacaggcatggcaagagtggctcccagtccccaccaggaacagggtgtgtggacactggtgcctgccttactgatcagttcataccttctgccaaggattccaattcgtccaaaagagattgaaccagtctgctaagagcctggacgtgcagcctatcctggttcctcttccacccccacatagaagcaggaaagacattagttcgaaatagatacaacagcccaagagatgaggctgagcccagcgtcaagggaatcaggagctactagagacagagggacagagaagagggagggagacagatggaaggacctgtaccaggagttatgggcacagaaaagaacatgaagacacagagaggaaggagagagataagacaccagcgaggggaagcctcactcattctaggtgccatggatgggatgataaagagagatgccttctaaagtcacaacttctcttcctaggagtccacagaaaaccttccctcctggcccacccaggtcccctggtgaaatcagaagagacagtcatcctgcaatgttggtcagatgtcatgtttgagcacttccttctgcacagagaggggaagtttaatgacactttgcgcctcactggagagctccatgatggggtctccaaggccaacttctccatcggtcgcatgacgcaagaccttgcagggacctacagatgctacggttctgttcctcattccccctatcagttgtcagctcccagtgaccctctggacatcgtgattacaggtgagagtgtctggacattattctcattgtcactgggacacagagtgaatgatccacgacttggaggcccaggtggttataaggaagatgagcttggtattcttatggagagagactaacttggtgaggtctgtaccaacagagacagagaaacaggagacacaagtacagaccaggtgtcataacagaggacagacacaggggccatacagggagttagaaaagacagaaagagttaaaggagacacagacagacatgtgccagagagaggtgtccttccatgctgactttgctcagagacctggcacaggttagaagtttcatttctgttttacttccacaaagtgttctctaccagaagaacccaaggacacccatatttctggcctgagttgggccctgtggcctcaggccttctggcacctacagatgccgtgtttattctgacacctctgccttccatgcaatggagagtaatcgtcccaggatatcatggccc

SEQ ID NO: 23 - protein, 233 aa, Homo sapiens
  MISC_FEATURE (1)..(233): Accession no AAB17133, NKG2A
MDNQGVIYSDLNLPPNPKRQQRKPKGNKSSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 24 - protein, 231 aa, Homo sapiens
  MISC_FEATURE (1)..(231): Accession no CAA04922, NKG2C
MSKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNSSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 25 - protein, 216 aa, Homo sapiens
  MISC_FEATURE (1)..(216): Accession no CAA04925, NKG2D
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 26 - protein, 240 aa, Homo sapiens
  MISC_FEATURE (1)..(240): Accession no CAA04923, NKG2E
MNKQRGTFSEVSLAQDPKPQQRKPKGNKSSISGTEQEIFQVELNLQNASLNHQGIDKIYDCQGLLPPPEKLTAEVLGIICIVLMATVLKTIVLIPFLEQNNSSPNTRTQKARPCGHCPEEWITYSNSCYYIGKERRTWEESLQACASKNSSSLLSIDNEEEMKFLASILPSSWIGVFRNSSHHPWVTINGLAFKHEIKDSDHAERNCAMLHVRGLISDQCGSSRIIRRGFIMLTRLVLNS

SEQ ID NO: 27 - protein, 158 aa, Homo sapiens [sequence omitted]

SEQ ID NO: 28 - protein, 201 aa, Homo sapiens
  MISC_FEATURE (1)..(201): Accession no NP_667341, NKp30
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 29 - protein, 276 aa, Homo sapiens
  MISC_FEATURE (1)..(276): Accession no CAB39168, NKp44
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTPWTPRDLVSSQTQTQSCVPPTAGARQAPESPSTIPVPSQPQNSTLRPGPAAPIALVPVFCGLLVAKSLVLSALLVWWGDIWWKTVMELRSLDTQKATCHLQQVTDLPWTSVSSPVEREILYHTVARTKISDDDDEHTL

SEQ ID NO: 30 - protein, 304 aa, Homo sapiens
  MISC_FEATURE (1)..(304): Accession no NP_004820, NKp46
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 31 - protein, 348 aa, Homo sapiens
  MISC_FEATURE (1)..(348): Accession no NP_055033, KIR2DL1
MSLLFVSMACVGFFLLQGAWPHEGVHRKPSLLAHPGPLVKSEETVILQCWSDVMFEHFLLHREGMFNDTLRLIGEHHDGVSKANFSISRMTQDLAGTYRCYGSVTHSPYQVSAPSDPLDIVIIGLYEKPSLSAQPGPTVLAGENVTLSCSSRSSYDMYHLSREGEAHERRLPAGPKVNGTFQADFPLGPATHGGTYRCFGSFHDSPYEWSKSSDPLLVSVTGNPSNSWPSPTEPSSKTGNPRHLHILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 32 - protein, 348 aa, Homo sapiens
  MISC_FEATURE (1)..(348): Accession no NP_055034, KIR2DL2
MSLMVVSMACVGFFLLQGAWPHEGVHRKPSLLAHPGRLVKSEETVILQCWSDVRFEHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHECRFSAGPKVNGTFQADFPLGPATHGGTYRCFGSFRDSPYEWSNSSDPLLVSVIGNPSNSWPSPTEPSSKTGNPRHLHILIGTSVVIILFILLFFLLHRWCSNKKNAAVMDQESAGNRTANSEDSDEQDPQEVTYTQLNHCVFTQRKITRPSQRPKTPPTDIIVYAELPNAESRSKVVSCP

SEQ ID NO: 33 - protein, 341 aa, Homo sapiens
  MISC_FEATURE (1)..(341): Accession no NP_056952, KIR2DL3
MSLMVVSMVCVGFFLLQGAWPHEGVHRKPSLLAHPGPLVKSEETVILQCWSDVRFQHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSETGNPRHLHVLIGTSVVIILFILLLFFLLHRWCCNKKNAVVMDQEPAGNRTVNREDSDEQDPQEVTYAQLNHCVFTQRKITRPSQRPKTPPTDIIVYTELPNAEP

SEQ ID NO: 34 - protein, 455 aa, Homo sapiens
  MISC_FEATURE (1)..(455): Accession no NP_006728, KIR3DL2
MSLTVVSMACVGFFLLQGAWPLMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHRDGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFRALPCVWSNSSDPLLVSVTGNPSSSWPSPTEPSSKSGICRHLHVLIGTSVVIFLFILLLFFLLYRWCSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 35 - protein, 489 aa, Homo sapiens
  MISC_FEATURE (1)..(489): Accession no NP_006854, LILRA1
MTPIVTVLICLRLSLGPRTHVQAGTLPKPTLWAEPGSVITQGSPVTLWCQGILETQEYRLYREKKTAPWITRIPQEIVKKGQFPIPSITWEHTGRYRCFYGSHTAGWSEPSDPLELVVTGAYIKPTLSALPSPVVTSGGNVTLHCVSQVAFGSFILCKEGEDEHPQCLNSQPRTHGWSRAIFSVGPVSPSRRWSYRCYAYDSNSPHVWSLPSDLLELLVLGVSKKPSLSVQPGPIVAPGESLTLQCVSDVSYDRFVLYKEGERDFLQLPGPQPQAGLSQANFTLGPVSRSYGGQYRCSGAYNLSSEWSAPSDPLDILIAGQFRGRPFISVHPGPTVASGENVTLLCQSWGPFHTFLLTKAGAADAPLRLRSIHEYPKYQAEFPMSPVTSAHSGTYRCYGSLSSNPYLLSHPSDSLELMVSGAAETLSPPQNKSDSKAGAANTLSPSQNKTASHPQDYTVENLIRMGIAGLVLVVLGILLFEAQHSQRSL

SEQ ID NO: 36 - protein, 466 aa, Homo sapiens
  MISC_FEATURE (1)..(466): Accession no AAH27916, LILRA2
MTPILTVLICLGLSLGPRTHVQAGHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTLGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSASLGQHPQDYTVENLIRMGVAGLVLVVLGILLFEAQHSQRSLQDAAGR

SEQ ID NO: 37 - protein, 420 aa, Homo sapiens
  MISC_FEATURE (1)..(420): Accession no AAM18036, LILRA3
MTPILTVLICLGLSLDPRTHVQAGPLPKPTLWAEPGSVITQGSPVTLRCQGSLETQEYHLYREKKTALWITRIPQELVKKGQFPILSITWEHAGRYCCIYGSHTAGRSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDAGYDRFVLYKEWGRDFLQRPGRQPQAGLSQANFTLGPVSRSYGGQYTCSGAHNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVS

SEQ ID NO: 38 - protein, 420 aa, Homo sapiens
  MISC_FEATURE (1)..(420): Accession no AAM18037. LILRA3
MTPILTVLICLGLSLDPRTHVQAGPLPKPTLWAEPGSVITQGSPVTLRCQGSLETQEYHLYREKKTALWITRIPQELVKKGQFPILSITWEHAGRYCCIYGSHTAGRSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDTGYDRFVLYKEWGRDFLQRPGRQPQAGLSQANFTLGPVSRSYGGQYTCSGAYNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVS

SEQ ID NO: 39 - protein, 191 aa, Homo sapiens
  MISC_FEATURE (1)..(191): Accession no AAF22159, LLT1
MHDSNNVEKDITPSELPANPGCLHSKEHSIKATLIWRLFFLIMFLTIIVCGMVAALSAIRANCHQEPSVCLQAACPESWIGFQRKCFYFSDDTKNWTSSQRFCDSQDADLAQVESFQELNFLLRYKGPSDHWIGLSREQGQPWKWINGTEWTRQFPILGAGECAYLNDKGASSARHYTERKWICSKSDIHV

SEQ ID NO: 40 - protein, 499 aa, Homo sapiens
  MISC_FEATURE (1)..(499): Accession no NP_036408, LILRA4
MTLILTSLLFFGLSLGPRTRVQAENLPKPILWAEPGPVITWHNPVTIWCQGTLEAQGYRLDKEGNSMSRHILKTLESENKVKLSIPSMMWEHAGRYHCYYQSPAGWSEPSDPLELVVTAYSRPTLSALPSPVVTSGVNVTLRCASRLGLGRFTLIEEGDHRLSWTLNSHQHNHGKFQALFPMGPLTFSNRGTFRCYGYENNTPYVWSEPSDPLQLLVSGVSRKPSLLTLQGPVVTPGENLTLQCGSDVGYIRYTLYKEGADGLPQRPGRQPQAGLSQANFTLSPVSRSYGGQYRCYGAHNVSSEWSAPSDPLDILIAGQISDRPSLSVQPGPTVTSGEKVTLLCQSWDPMFTFLLTKEGAAHPPLRLRSMYGAHKYQAEFPMSPVTSAHAGTYRCYGSRSSNPYLLSHPSEPLELVVSGATETLNPAQKKSDSKTAPHLQDYTVENLIRMGVAGLVLLFLGILLFEAQHSQRSPPRCSQEANSRKDNAPFRVVEPWEQI

SEQ ID NO: 41 - protein, 368 aa, Homo sapiens
  MISC_FEATURE (1)..(368): Accession no NP_077293, LILRA5
MVSILTVLLCLRLSLGQKAQALAGTLPKPSLWAEPGSVITWESPMTLWCQGTLDTQGYYLTKEGNPMTWYQQSPPEPRNKTNFFIPSMREHHAGRYHCHYLSPAGWSERSEPLELVVTGVSRKPSLLTLQGPVVAPGENLTLQCGSDVGYDKFTLYKEGGHDLVQGSGRQPQAGLSQANFTLGPVRVSHGGQYRCYGAHNLSSEWSAPSDPLSILIAGQIRGRPSLSVQPGPTVASGENVTLLCQSREQLDTFLLTKEGAAHHPLRLRSEHQAQQHQAEFPMSPVTSAHAGTYRCYSSRRFFPYLLSHPSDPLELVVSGAAETLSPSQNKTDSKTASPASHPQDYTVENLIRVAVAGLVLVVLGILLL

SEQ ID NO: 42 - protein, 464 aa, Homo sapiens
  MISC_FEATURE (1)..(464): Accession no AAD02204, ILT8
MTPILTVLICLGLSLGPRTHVQAGPFPKPTLWAEPGSVISWGSPVTIWCQGSLEAQEYRLDKEGSPEPWDRNNPLEPKNKARFSIPSITEHHAGRYRCHYYSSAGWSEPSDALELVMTGAYSKPTLSALPSPVVASGGNMTLQCGSQKGYHQFVLMKEGEHQLPRTLDSQQLHSGGFQALFPVGPVNPSHRWRFTCYYYYMNTPRVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSFPSEPLELMVSASHAKDYTVENLIRMGMAGLVLVFLGILLFEAQHSQRNPQDAARR

SEQ ID NO: 43 - protein, 368 aa, Homo sapiens [sequence omitted]

SEQ ID NO: 44 - protein, 299 aa, Homo sapiens
  MISC_FEATURE (1)..(299): Accession no AAK52451, ILT11
MAPWSHPSAQLQPVGGDAVSPALMVLLCLGLSLGPRTHVQAGNLSKATLWAEPGSVISRGNSVTIRCQGTLEAQEYRLVKEGSPEPWDTQNPLEPKNKARFSIPSMTEHHAGRYRCYYYSPAGWSEPSDPLELVVTGFYNKPTLSALPSPVVTSGENVTLQCGSRLRFDRFILTEEGDHKLSWTLDSQLTPSGQFQALFPVGPVTPSHRWMLRCYGSRRHILQVWSEPSDLLEIPVSGAADNLSPSQNKSDSGTASHLQDYAVENLIRMGMAGLILVVLGILIFQDWHSQRSPQAAAGR

SEQ ID NO: 45 - protein, 243 aa, Homo sapiens
  MISC_FEATURE (1)..(243): Accession no  AAF73849, ILT11
ISRGNSVTIRCQGTLEAQEYRLVKEGSPEPWDTQNPLEPKNKAGFSIPSMTEHHAGRYRCYYYSPAGWSEPSDPLELVVTGFYNKPTLSALPSPVVTSGENVTLQCGSRLRFDRFILTEEGDHKLSWTLDSQLTPSGQFQALFPVGPVTPSHRWMLRCYGSRRRILQVWSEPSDLLEIPVSGAADNLSPSQNKSDSGTASHLQDYAVENLIRMGMAGLILVVLGILIFQDWHSQRSPQAAAGR

SEQ ID NO: 46 - protein, 234 aa, Homo sapiens
  MISC_FEATURE (1)..(234): Accession no NP_061113, TREM1
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 47 - protein, 230 aa, Homo sapiens
  MISC_FEATURE (1)..(230): Accession no NP_061838, TREM2
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 48 - protein, 200 aa, Homo sapiens
  MISC_FEATURE (1)..(200): Accession no NP_937796, TREM4
MAWGGVHTCCFHLCCCCSWPQGAVPEELHKHPGQTLLLQCQYSPKRGPYQPKSWCQQTSPSRCTLLVTSSKPWTAVQKSHYTIWDKPNAGFFNITMIQLTQNDSGFYWCGIYNASENIITVLRNISLVVSPAPTTSPMWTLPWLPTSTVLITSPEGTSGHPSINGSETRKSRAPACLGSGGPRFLVLVLCGLLLAKGLML

SEQ ID NO: 49 - protein, 254 aa, Homo sapiens
  MISC_FEATURE (1)..(254): Accession no AAH17865, FCGR3A
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 50 - protein, 1593 aa, Homo sapiens
  MISC_FEATURE (1)..(1593): Accession no NM_014218, KIR2DL1
CGCGTGGGCGTGCCTGTCTGCTCCGGCAGCACCATGTCGCTCTTGTTCGTCAGCATGGCGTGTGTTGGGTTCTTCTTGCTGCAGGGGGCCTGGCCACATGAGGGAGTCCACAGAAAACCTTCCCTCCTGGCCCACCCAGGTCCCCTGGTGAAATCAGAAGAGACAGTCATCCTGCAATGTTGGTCAGATGTCATGTTTGAACACTTCCTTCTGCACAGAGAGGGGATGTTTAACGACACTTTGCGCCTCATTGGAGAACACCATGATGGGGTCTCCAAGGCCAACTTCTCCATCAGTCGCATGACGCAAGACCTGGCAGGGACCTACAGATGCTACGGTTCTGTTACTCACTCCCCCTATCAGGTGTCAGCTCCCAGTGACCCTCTGGACATCGTGATCATAGGTCTATATGAGAAACCTTCTCTCTCAGCCCAGCCGGGCCCCACGGTTCTGGCAGGAGAGAATGTGACCTTGTCCTGCAGCTCCCGGAGCTCCTATGACATGTACCATCTATCCAGGGAAGGGGAGGCCCATGAACGTAGGCTCCCTGCAGGGCCCAAGGTCAACGGAACATTCCAGGCTGACTTTCCTCTGGGCCCTGCCACCCACGGAGGGACCTACAGATGCTTCGGCTCTTTCCATGACTCTCCATACGAGTGGTCAAAGTCAAGTGACCCACTGCTTGTTTCTGTCACAGGAAACCCTTCAAATAGTTGGCCTTCACCCACTGAACCAAGCTCCAAAACCGGTAACCCCCGACACCTGCACATTCTGATTGGGACCTCAGTGGTCATCATCCTCTTCATCCTCCTCTTCTTTCTCCTTCATCGCTGGTGCTCCAACAAAAAAAATGCTGCGGTAATGGACCAAGAGTCTGCAGGAAACAGAACAGCGAATAGCGAGGACTCTGATGAACAAGACCCTCAGGAGGTGACATACACACAGTTGAATCACTGCGTTTTCACACAGAGAAAAATCACTCGCCCTTCTCAGAGGCCCAAGACACCCCCAACAGATATCATCGTGTACACGGAACTTCCAAATGCTGAGTCCAGATCCAAAGTTGTCTCCTGCCCATGAGCACCACAGTCAGGCCTTGAGGGCGTCTTCTAGGGAGACAACAGCCCTGTCTCAAAACCGGGTTGCCAGCTCCCATGTACCAGCAGCTGGAATCTGAAGGCATGAGTCTGCATCTTAGGGCATCGATCTTCCTCACACCACAAATCTGAATGTGCCTCTCACTTGCTTACAAATGTCTAAGGTCCCCACTGCCTGCTGGAGAAAAAACACACTCCTTTGCTTAGCCCACAGTTCTCCATTTCACTTGACCCCTGCCCACCTCTCCAACCTAACTGGCTTACTTCCTAGTCTACTTGAGGCTGCAATCACACTGAGGAACTCACAATTCCAAACATACAAGAGGCTCCCTCTTAACGCAGCACTTAGACACGTGTTGTTCCACCTTCCCTCATGCTGTTCCACCTCCCCTCAGACTAGCTTTCAGTCTTCTGTCAGCAGTAAAACTTATATATTTTTTAAAATAACTTCAATGTAGTTTTCCATCCTTCAAATAAACATGTCTGCCCCCATGGT

SEQ ID NO: 51 - DNA, 918 bp, Homo sapiens
  misc_feature (1)..(918): Acccession no AF097358, MAFA mRNA
cacgagcttagctgaagatgactgacagtgttatttattccatgttagagttgcctacggcaacccaagcccagaatgactatggaccacagcaaaaatcttcctcttccaggccttcttgttcttgccttgtggcaatagctttggggcttctgactgcagttcttctgagtgtgctgctataccagtggatcctgtgccagggctccaactactccacttgtgccagctgtcctagctgcccagaccgctggatgaaatatggtaaccattgttattatttctcagtggaggaaaaggactggaattctagtctggaattctgcctagccagagactcacacctccttgtgataacggacaatcaggaaatgagcctgctccaagttttcctcagtgaggccttttgctggattggtctgaggaacaattctggctggaggtgggaagatggatcacctctaaacttctcaaggatttcttctaatagctttgtgcagacatgcggtgccatcaacaaaaatggtcttcaagcctcaagctgtgaagttcctttacactgggtgtgtaagaaggtcagactttgatagatgaccactctgtcctgaccctcagatctgtcatgtatccctaaaaggagggagctggccactggctgttgggaaagccatgagtatatagttagcaaatactgaactttctcagatatggcattagatgcaagacaacctcctagggattgatgcctaactgatggattctctttgagactatttagatattatgtgagcaatttaaagaccagatctaagcaaattttgaaatagatgtttgttttttgtatttctcagtatggaaactaatgctgccactctcatccccgtcccaaccatactctgtcaaaaatatacctttttc

SEQ ID NO: 52 - protein, 189 aa, Homo sapiens [sequence omitted]

SEQ ID NO: 53 - DNA, 1135 bp, Homo sapiens
  misc_feature (1)..(1135): Accession no NM_004829, NKp46 mRNA
aggccggcagaatctgagcgatgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcggatgggcctggcctttctagtcctggtggctctagtgtggttcctggttgaagactggctcagcaggaagaggactagagagcgagccagcagagcttccacttgggaaggcaggagaaggctgaacacacagactctttgaagaatgaccatgagacacagtggccatgggtggatctgaaagctggtgttgagcctgggcggcgtgagctctgtgttggacccacggaggagggagtcactgcagggaaagagggacactggcattccatttgtcagagcatcccggacgatgcagagggtgggagaactacatgctaaatttctttttttttttttttg

SEQ ID NO: 54 - DNA, 23439 bp, Homo sapiens
  misc_feature (8130)..(8132): Start codon ATG
aggggacgggcttaccttccactgtgagagttacctagactgtgatggtcctgtaggcttctgaggcgatcgggatcgggcagtgtcagtcttcagctgctaagccgagaagatctgggaaggagtcagagagccttgggccagagttctagctgctctgggagtggctgccaggtgagttgaacagtccgattttcagtggggtcccgcacagatgggatgcggcttaggaggaatcccaggctgtggacattccttggcccagtggccagatttccagtacttgtagcaagctcctgggggaagaggttctggaggaacccctggcagctgcggttcaggcgtttggagttctcgtgtgctggagatgtggctggggtttgtctcatctggatactggagtggaggcaaggaattgcaactcagaaatatgttgctatttggctgcctctactctattactgtacaccttgaaggcgaggttaattaagtcttgttgtggggtttgagggacagaatttaatttttggagctttatttaatgttgggagcagatttggtaataaaatgtatattgagaataagacggccttttgacttagggtctagggctgtaaagcgtctcagggttgctgccaaatgagccatgaactgggctgtgtttttaaatttgatgaaaaagagcctaaacactatctgatttgggagaggtcagataaagaaaaaggagcattaaccttgactatgcctttagcttcagccacctttttaagaggaaattgctgggcagttgggggagggctagtcatggaatggaactgtaagctggaccgggtgtgaggaggggaggtgataaaaggattatagggtggaggagcggaggctgaggaagaattgggacccagctcggcctggcgaggaggggagatgtcagatgggtctgtagaaaaggaagattagaaagactcagcgatgcttggggttgggactgacgggacaggcgggagggaaagaaggaagatttgggacgagttgcactgggcatagagactagggagggaccgatgtgtaaaagaatgcctggatgtcaggcacctcagaccatttgcccattttacaacaagaattatttagatcttgtaggatggaaaaattgaaagtgccgttttctggctatttggaaccactgtcaagtttgtattggggtcaagcagcattgcagaagaaaataaggcatttaggttttaggtcaggtgtgagttgaagaggttttaggtttttaagaacacaggctaagggagaagaaggaggaatggagggtggaaggttgcccatactgaaggaggcaagcacagagaaaagagagagtagagacatggagggaaggggttcaggggttcttaccttccagaaaagcgggaaaggggtcagggcacagaagtaagggattggggtgcagagacaagaggtcggggtgtggaaataagggatcggggtgcagagataagacgtcagggcacagaaataagggatcgggggattcttgccccctagaaaagcggtacttgccactaagggtgaaggagaaggggttggggggttcttgcccccccagaaaagcagagaaggggtagagacacagagaaggagttgggggttcttgcccccccagaaaagcagtacttgccactaagggtgaaggaccaaggcaggcatccccatgtggtcagacacctctgaaacgtgggtgaataatcagagaggtgtccctgcgtgattaaacaccaagggaaggctgccttcccgagtccatgaccggcgctggagttttgggtccacgaataaagcgcgtctcctgtctctaccagaaaaggaaaggaactgaaattaagagaagggagagattgaagagtggaaaggagaaagtggttgagggatagtgagagaggttggagaagagagtaaaaagaggctgcttactggatttaaaattggtgagatgttccttgggctggttggtctgaggacgagaggtcgtaggtggatctttctcatggagcaaagagcaggaggacaggggattgatctcctaaggaagatcccctgattcgagttatggcaccaaatttcactcacgtccgtgtgaagagaccaccaaacaggatttgtgtgagcaacaaggctgtttatttcacctgggtgcaggcgggctgagtccagaaagagagtcagcaaagggagataggagtgcggccgttttataggatttgggtaggtaaaggaaaattacagtcaaaagggggttgttctctggcgggcaggagtggggttcacaaggtgctcagtaggggagcttttgagccgggatgagccaggagaaggaatttcataagataatgtcatcacttaaggcaagaacaggccattttcatttctttcgtggtggaatgtcatcagttaaggcaggaaccggccatctggatgtgtacatacaggccacagggggatatgatggcttagcttgggctcagaggcctgacagtctggatcacctgacctggtgatccgcacacctcggcctcccaaagtgctgggattacaggcatgacccactgcacctggccttagaaaacttcttaaatattaaaatgtatgttatgtgtattttgccacaatttttgaaaagtaccttctggtgtttagagacagaagatgagtggttgcctagggccgggagagtgaggggatcgtggtgatgggcagctggtcggcatggggttctgaagggcagtgatgacaacattctaaaattagactgtgttgacggttgcaccaactccgtgaataccacaaaatttaaaccattgaattatgcacttttaatgggtaattgtatggcatgtaaattatatctcaataaagttatatttttaaataccaaaaaaaggccgggtgcggtggctcacgcctgtaatcccagcactttgggaggccgagaagggcggatcacgaggtcaggagatggagaccatcctggctaacatggtgaaaccccatctctactttgaaaaaaaaaaaaaaaaaaaaaagattacccggacgtggtggtgggcacctgtagtcctagctactcaggaggctgaggcaggagaatggcataaactcgggaggcagagcttgcagtgagtcgagattgcgccactcaggaggctgaggcaagagaatggcataaaccccggaggcagagcttgcagcgagccgagattgcgccactgcactccagcatgggtgacagagcaagagtccatctcaaaaaaaaaaaaaaaaagattagtaatatcctctgtgtcacttaccacttaagtgattgaatcacgacttgaaattcatcatctcaaacatggcttagagtctgtagaggggggacagtcccaggaatgctggtgtgggcttaaggctgaattaaatagatccagatggctcacacctgtaatcccaataccttgggaggccgaggcaggtgggaggctgaggcaggcggatcactggagctcctggagcgaagaaaggatgctagtggaaaaactggtgaaatcagaataaagtctatagttttattttttaaaggaggctgggcgtggtggctcatgcctctaatcccagcactttgggaggctgaggcaggtggatcagttgagttcaggagttcgaaaccagcctggccaacttgacgaaaccccatctctactagaaatacaaaaattagctgggcgtggttgtgggtgcctctaatcccagctactcaggaagctgaggcaggagaattgcttgaacccaggaggcggaggttgcagtgagctgagatcacaccattgcactccagcctgggctacagagcaagattccatctccaaataagagagacatgacaattaaataaattgtgtaatcttggattaaatcctaaaccaaatatatgtcactggtaaaacaagtggtgaaatttgaataaagtggatagatcagacaatagtgtcatatcagtgctatttcttgaccttgaacattaataacagaatgtccttggttttgggaaatataacctgaagtgattagaggtttagggcatcatatgcaaattagacacactttcttcggggagagagggagagggagagaggctgaatgatgaagcaaatgtggtaaaatgctaactttggggaaatctggatgaagaaattacagattttttttttttttatagacagggtaacactctgtcacccaggctagagtgcagtggcacgatcatggctcactgcagcttctacctccctgggctcagatgaccctctcacctcagcctcccaagtagctgggactataggcgcacagcaccacacctggctaatttttgcatttttttttcccccaggctcgtctcaagcaatccacccacctcggcctcccaaagtgctgggattacaggtgtgagccactgcacctggccagaaattctttaaactatttttgcaagtctggaattatgtcaaaattaaaagctcaaaataataaaagacaatattcttatatttctttggtgaaggtaactatgttatggctgagagggtggctgaggtctgaggatccagcctacataagtctcctccatagagggcatccaagcgctccgtagggggaaggataaagaaaacacccagagttatgacagctgtgtaaggggaaacgccagcaccgagtactgaatcttcagtaaataagaaggaggcgggctgggtgtggtggctcacgcctgtaatcccagcactttgggaggctaaagtgggctgatcacttgaggtcaagagttcgagactagcctggccaacatggggaaaccctgtctctactaaaaatacaaaaattagtcgagtgtggtggcacacgcctgtaatcccagctacttgggaggctagaacaggagaattgcttgaacccaggaggtgaaggttgcagtgagctgagattgcaccactgcaccccagcttgagggacagagtgagattccgtcttaaagagaaaaaaaaaagaattagcacatttgtttgcctcaagaagatacaactagtcttgtacagtagtcacatgtatccaccaggatatattccaaggccccagtggatgctgaaaactacatagtaccttacatgtatatatatatgtatatacatatatacacatatacgtatatgtatacatacatgtatatatgcatgtatgtatatacatatatgcatatatacatacatgtatatatacatgtatgtatatacatatatgtatatgtatgtatacacgcatacatgtatgtatacacgcatatatgtatgtatatacatatatgtatgtatacacgcatacatgtatgtatatacatatatgtatgtatacacgcatacatgtgtgtatacatatatatgcatgtatgcatgtgtgtatatatacatatatgtgtatatatacgcatatacatgtatgtgtatatatgcatgtgtatatatacatgtacggtactatgcagtatatatacacatatatgtatatatgtatacatatatgtataaatgtatatatgtgtatatatataaaaggtatatatgtatatatgtgtgtatatataaaatgcatgaatttcttttttcttactgtagatcttaacaacttctgcatagaatttttttttattaagtggagagttagttacttacttaaaagaaatgtttcttggctgggtgtggtggctcacacctgtaatcccagcactttgagaggccgaggcaggaagattcacttgaggtgaggagttggagaccatcctggccaacgtggtaaaaaccggtctctactaaaagtacaaaaatgagctgggcgtggtgttgggtgtctgtagtcccagctactcaggtggctgaggcaggagaattgcttgaacccacaaggcagaggttgcagtgagctgagatcacaccactgcaccacagcctgggcaacagagcaagactctgtctcaaaaaaaaaaaaaaaagaaagaaaaagaaaaagaaaagaaatgtttcttttcttattaagttctttaaatgaaaagcttttcttttcacttttattttattgaaacattataacactatctttgaagaagttagtgttatcattccattctgatgaaaccaattaacttatccaagcatatgtatactgtacacagagaagccaacgtcaaaacccctatttttatctttttagattcagcagatacatgtgcaggttttttatgagtatattgcatgatgctgaggcttgcattaatgatctagtcaccaaataggtagattttcaagccttgctcccctccttacccaatgtttagcgctctcacttataagtgagaacatgtggtatttggttttcttttcttttttttttttttttttgagatggagtttcactcttgttgcccaggctggagtacaatggcaccatctcggctcactgcaaccttcaccttccaggttcaagcaattctcctgcctcagcctcccgagtagttgggactacaggcatgtgccaccacacccggctaattttgaatttttagtagagacagggtttctgcatgttggtcaggctggtctcgaactcccgacctaaggtgatccacctgcctcagcctcccaaagtgctgggatgacaggcgtgagccaccgtgtctggccagtatttggttttctgtttctgtgttaactcgcttaggataatggcctctagctgcatccatgttgctgcaaaggacataatcttgtgatttttcaaggctgtatagcgttctgtggtgtatacatatcacattgtctttatccagtccacctctgatgggacctgggtggattccatgtcttcactattgtgaatcctgctgcaatgaacatacaagtgcatgtgtctttttggtagaatgatttattttcctttggctatatacccagcgatgggattgctgggctgaatggtaactctgtttgtagttctctgaaatatctccaaaccaaactgctttccacagtggctgaactaatttacacccaccaacagtgtataagtgtcccctttgctccacaatctcaccagcatctgttaatttctggcttttcagtaatggccattctgactggtgtgagatggtattgttgagggataatttaggaatcagagagaccgaggggttgaggaggatttattattattattattatttaggtgcaccggccccagtcagattaacatccaaaaagactgaggctcgaacagagagtccggttaccttttaagcattttgtggggttgggggagatctgtgcagggggaagcatattacagaagcaagaaacaaaggcagttattcaattgagacatgcatcacattattccttacttttcaagaaaaatatgttttacgacttgaggttatcctgtctagtgatcttgcagccgcacggcaagagaaacagggtcttcacaatgcctgggaaagggagagataaggctcactagccacagacagaaaaacaggcagttcatgtttaaaggactccacctctttctcttcctcggggggaactgggttttcttaaatacaactgagtttttgtttacacattctgtaatttcttttaattcctgttccagtatctcactgtgaaactccctatgtttttatacgattctcagggggtttcctctgggcatgattgggcacaacttcccacagtcagctctgggtacgacctccacattgcagaattgagaagttgacccagaaatgcattttgggctgagcagacaattgtcagagttgctggctagaccacagatgtgtcagagggaccacggcctttctgtaagctcatggtcagaggcggaggggagttgtgaacgttctgatgaaagcagtcaacgtgaaagcgctctggtgatgggcgctggtgctcacccaccacttcctgtgtatctatctccctggcccgcccggctcagtccccactgctcagcactaggccggcagaatctgagcgatgtcttccacactccctgccctgctctgcgtcggtgagttctggcgtggaaggggaatgggatcacggtgtgcctgggaggcaacaggtctcattactcccgtcttccagggctgtgtctgagtcagaggatcagcgcccagcagcgtgagtccttccttcaaagcccagggtcactcttccgggttcaggccaagctccttccacccaagcacggctggggagaggggacagggtgctggcttcccaggagagcttggggccagcagctgggtggagcctaaggttggggggagggggctccgctggaactccagcctctgattcccttccagagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacaggtaactgtccggttctctaactggagagtgatctcagtctgcatccgggatgcagcatcatctatgaactcttccaagccccactcagacactgcttgtctcggtaggaggctggaaggaggggtgatccccatcacaatccttgcctacaaggggttgtctgcagaccgtgtctctacgtcctaggagcagatgtgtcctcagtcagtttctccatgacacagattctgagatagatatttgtatgcaggggtatgactgaggaatgtcctcaaaaacaatgcctgtgggctaggcgcagtggcttacactttgcttccctcacccatcacaggtggtgggttttttttttttttatctgttttgagacggagtttcgctcttgtcacccaggctggagtgcagtggtgcaatctccagtcactgcaacctccacctcctgggttcaagtgattctccagcctcagcttcccaagtagctgggatcacaggcacccaccactacgccacattttgtatttttagtagagatggggtttcaccatgttggccagggtggtgtcgaactcctgacctcagatgatccgcccgcctcaccctcccaaagtgctgggattacaggtgtgagccatcacacccagccaggtggtggttttctaaaaaaaaaaaaaaaaattagctttttttttttttaacaatatggttgtttattattattatcaagtattatacatagttacatatacatacataattgtatgtgctatacaattaggtttgtttataccagcaacaccaaaaacacatgagcaatactttgtgctaggaaggctatgatgtcatcaggcaataggaatttttcagtttcattataatcttatgggaccaccatcatatatgtggtacattgttggccaaaatgtcattatgcagctcacaacagtatttcatgtccattcaaatatcttcttttgtgaaatgtctatttaaatcttttgcctatttttaaattgggttgcttatattttgattgattaggaaaagttatttctatattctgtgtcatatacttgtgttgaaatatatatattttttgtctgtgccttttcatttgctcagggtctttggaccttgtttggaggttctggcaggggaacacagctactcatttattcttttttttttaatttttttagtatttattgatcattcttgggtgtttctcggagagggggatttggcagggtcataggacaatagtggagagaaggtcagcagataaacatgtgaacaaaggtctctggctttcctaggcagaggtccctgcggccttccgcagtgtttgtgtccctgggtacttgagattagggagtggtgatgactcttaaggagcatgctgccttcaagcatctgtttaacaaagcacatcttgcaccgcccttaatccatttaaccctgagtggacatagcacatgtttcagagagcacggggttgggggtaaggtcatagattaacagcatcccaaggcagaagaatttgtcttagtacagaacaaaatggagtctcctatgtctacttctttctacacagacacagtaacaatctgatctctctttcttttccccacatttcccctttttctattcgacaaaaccgccatcgtcatcatggcccattctcaatgagctgttgggtacacctcccagacggggtggcggccgggcagaggggctcctcacttcccagacggggcggccgggcagaggcgcccccccacctcccagacggggcagtggccgggcgggggctgccccccaacctcccggacggggcggctggccggggctttttttttttttttttgagacagtctcgctgcagtgcagtggtacaatctcagctcactgcaacctctgcctcagcctcaattctcctgcctcagcctcccaagtagttgagattacaggcatgtgccaccacacccggctaatttttgcatttttagtagagacggggtttcaccatgttgaccaggctggtctcaaactcctgacccaggaggtcgagtcttcagtaagcaaagatagtgccacggcgctccagcctgggaaacagagcaagaccctgtatcatttttaaaaatggttttagacggtaaatcttctattgtgtgtatttgaccaaaataataattaaaaaaaaaaaaaaagctggctgccaggcatggtggcaggcccctgtagtcccagctacttgggagggtgaggcaggagaaacgcttgaacccgggaggcagaggttgcagtgagccaagatcgtgtcactgcactccagcctgggcgacagagagagactccatctctaaagaaagaaaaaaaaaaatagctggctgctcatcactgagtttctggtgtggtggccccaccttctctcatagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggtgaggaaatgctcaattccccacacccttcgccgccatgtcctacctggagccctgagggatccccagagagtgatggggagggtgtccaagggacgtccacttcctgggtgcctggttggtcatgtgaggaagaacaccagaagcaggaaggaggagggagcagagaaaggaatggtaaggcgggtggatcacaaggtcaggagttcgagaccagcctggccaagacggtgaaaccccgtctctactaaaaatacagaaattagccagacgcagtggcggacacctgtagtcccagctactcaggaggctgaggcaggagaatcgcttgaacccgggaggcgggggttgtagtgaaccgagatcataccaccgcactgcaacctgggcgacagagcaagactccatctcaaaaaaaaaaaaaaaaaaaaaaagaatggcaagaccggaggaaaccaaaaacccttacttttttttctttatctccttttccaggcgacattgagaacaccagccttgcacctgaagaccccacctttcctggtgagtaactggtccttctaagctcagacgagcgatcagagcctcccagtgacactaaaaacgtggcattcattcaaaatattcatcgaggccaggcgtggtggctcacgcctgtaatcccagcactttgggaggccgagatggtgcatcatttgaggtcaggagtttgagaccagcctggccaacatggcgaaaccctgtctctactaaaaatacaaaacttaggctgggcatcatggctcacacctgtaatcccaacacttcgggaggccaaggtggttggatcacaaggtcaggaattcgagaccagcctgaccaacatggtgaaaccccatctctactaaaaatacaaaaattagccgggcctggtggtgctcgcctgtaatcccagctactcaggaggctgaggcaggagaattgttgaacctgggatgcagaggttgcagtgagctgagatcgcgccactgcattccactccactgcacgacacagcgagactccatctcacagaaaaacaaaaacaaaactattatatatatatattcatcaagtgcatagtatacacagtgaactacactgtaacagtcagccaggcagatatcttgactctgcagcacttagattctagcaggaggagacacaccatcggtcaacgtcaggatagcacacaggagggaatgatgctatggaaggaaaagacaaagtagaacagacttacagtgattgaaatggcagctagcaatattaaataggtttgtccagatggacctcacagagaaagaaggcatctgagcaaatgcgttcagacttgagttaatcatgtggctgtcaggagaaaggaggctctggagagaatgaaatggcatctgcctgtgccctggggcaggaagataactggggtaatacaataataactatgaggccaggagggttgaaaatgatgtttggaagatgacggtgggatgggcctggggcgcacggctaggattacaggagtgaggcccggcgcggtggctcacgcctgtaatcccagcactttgggaaaccgaggcaggtgggtcatgaggtcaggagatcaagaccatcctggctaacacggtgaaaccctgtctctactaaaaaaaaatacaaaaattatccgggcgtggtggcgggcgcctgtagtcccagctacacaagaggctgaggcaggagaatggcgtgaacccgggagacggagcttgcagtgagctgagatcgcgccactgcactccagcctgagcgacagagtgagactccgtctcaaaaaaaaaaaaaaagaaaaagaaaaagaaaaaaaaatagtgagactttgaatttcactatgtgtgtatgtgtgaggagaaagaggtaatgatgacttaatgaggaaaatgaggcttaaatagaagacgggctgggccgggtggctcccgcatgtaatcccagcactttggaaggcaggggcggctggatcacttgaggtcaggagttcaagaccagcctggccaacacagtgaaaccccatctctactaaaaatacaaacatgagttgggtgtggtggcgcacgccagtaattacagctactcggggctgaagcaagaggattgcttgaactcgggaggcggaggttgcagtgagctgagatcacaccactgtactccagcctcagaggcctgtcatcccagccctttgggaggccgaagcaggcaggtcatctgaggttgggagttcaagaccagcctggccaacatggcaaaaccccgtttctactaaaaatatgaaaaaaattacctgggtatgtggtgtgtgcctgtagtcccagctactccagaggctggaacacagtgagactctatctcaaaaaaaaaaaaaatagaagacatgactggtgcaaagacacatgctcacaagtgctagaatggaattcctcgtcaggttcgtccatctgtggacccttccactttacctgctggatgaagctcctgggacccgcagggtgaggtgggaccttgtaaagctgcagaacgtcatggggtagacccaagggaaggagtgctggggtggaggaggtcaaaaccatcctcttttcttcacttcccttatcatcagcagacacttggggcacctaccttttaaccacagagacgggactccagaaaggtaagtagacagctggggccataggctctgaaggaaggggctgggcatagagtagacctaggaagggaatctaaatgggaacaagagggtgtccttggccaggcgcagtagctcacacctgtaatctcagccctttgggaggccgaggcgggcagatcatctgaggtcgggagttcaagaccagtctggccaacatggcgaaatcccatctctactaaaaatacaaaaaaattagccaggcgtggtggcgtgtgcctgtagtcccagctacttgggaggctgagacaggagaatagcttgaacccaggaagtggaggttgcagtgagccgagatcgtgccattgcactccagcctgggcgacaagactgaggctctgtctcaaaaaaaaaaaaaaaaaaaaaaaaaaaaaaaaaaaaaaagagggtgtccttacatccctgtcagcgatcaccctgttctcctgcctacagaccatgccctctgggatcacactgcccagaatctccttcggatgggcctggcctttctagtcctggtggctctagtgtggttcctggttgaagactggctcagcaggaagaggactagagagcgagccagcagagcttccacttgggaaggcaggagaaggctgaacacacagactctttgaagaatgaccatgagacacagtggccatgggtggatctgaaagctggtgttgagcctgggcggcgtgagctctgtgttggacccacggaggagggagtcactgcagggaaagagggacactggcattccatttgtcagagcatcccggacgatgcagagggtgggagaactacatgctaaatttctttttttttttttttgagacagagttttctcttgttgcccaggctggagtgcaatggcgcgatcttggctcactgcaacctctagctctccatccctcgggttcaagtgattctcctgcctcagcctcctgagtagctgggattacaggcatgtgccaccaccccagctaattttgtatttttagtggagacggggtttctccctgttggctggtctcgaactcctgacctcaagtgatctccccgccttggcctcccaaagggctgggattacaggcataagccgctgcgcccagccactgaatttcttctgtagacaaatcctatggtctcttctaggctctaactatttttgtaccacttactgcaaaccatacttttaaccactctggtcttttctgaaaagatctctccttctttaacaggatggccatggaaatatttttttcctactttggtctttttttctttcctttctctgcaggaagccattcaaaatagttaataaccaatatagaataggtctgtatcaaatggttcaggaggcattgtggcaacaaccagttgtagagaagcagctttataagtgaatcctgccaggcacggtggctcacacctgtaatcccaacactttgggaggctgaggcgggcagatcacctgaggtcaggagttcgagaccagcctggccaacatgatgaaaccccatctctactaaaaatacaaaaactcggccaggcacggtggctcatgcctgtaatcccagcactttgggaggccaaggtgggaggatcacctgaggtcaggagttcgagagcagcctggccaacatggtgaaaccacatctctactaaaaatataaaaattagccaggtatggtggcgtgtgcttgtaatcccagctactcaggaggctgaggcaggagaatagcttgaacccgggaggcggaggctgcagggagccaagatcgcaccactgcactccagcctacgtgacagagcaagattctgtctcaaaaaaaaaaagaaaaaaaaaaaataagtgactcctggctgcatcccaaccataccccaattccttctaaccacagaattattccatcttctcttccttttttttttttttctttttttttgtttgttttgttgggacagaatttcactttttttttttttaatgtaagttttagggtacatgtgcacaacgtgcaggttagttacatatgtatacatgtgccatgttggtgtgctgcacccactaactcgtcatttaacattaggtatatctcctaatgctatcccttcccccgagtttcacttttgtcacccaggctggaatgcagtggtgcaatcttggctcactgccacctccacctccagggttcaaatgattctcctgcctcagcctcctgaatagctgggattataggcatgcaccaccacgcccggctaatttttgtatttttagtagaaatggggtttcacaatgttggccagactggtcttgaactcctgacctcaggtgatccaccagcctcggcctcccaaagtgctggaattacaggtgtgagtcaccgtacccggccaccatctttgcttctttatccacaccttgccttgttcttcagggctctgcagagatatcatttcctccaagagtttccacaactccgacttcacaaagatagcacttttttttttttttttgagacagtctcactctgtagcccaagctggcgtgcagtggcacaatctcagctcactgcaaccttcgcctctggggctcaagcgattctccttcctcagcctcccaagtagctgggactagaggcgcgcgccaccacacccggttaattttttttgcatctttagtagaggtagggtttcatcatgttgccctgggtggtctcaaactcctgagttcaggtgatccccccgccttggcctctcaaagtgctaggattacaggcgtgagccactgcgcccagccaagacaacactttcctcatcccaaagcacctgttaattccctgtaacagcacttgaaccctgattcggcatgcatgtccattttcctgcctctaccgtgaactcgtgtgaattgatctatgtcagatttagtggctgcattcacagctcccgcaactataacggggttctcgggaaatatatatcaaatgagtgaatgtatatacggggctgtggcacagcctgcaacttgagacttctcactaggggtcttgaaatgctgtctggacaccaccatcgctttcctccctgagaacttctacttatcaacccatttatatactcatcgcatgggtcctcacgccctcccattattctggtgcctcatgccggtcaaatttattctctaaatctgatttttccattaaatagcagcctggccaacacggtaaaaccccatctctactaaaaaatacaaaatattagccaggcgcagtggcttgcacccgtaatctcagctactcgggaggctgaggcagcagaatcacttgaacccgggaggcagaggttgtggtaagccgagattgcaccactgcactccagcctggtaacagagcgagactccctctcaaaataaataaactgctgactcgcgtattttttctttaccccaactcattccttacatgtaggcacctgtaatcctagctactcagaaggctgaggcaggagaatcgcttgaacctgggaggcggaggttgcggtgagccaaaatcgtgccactgcactccagcctgggcgacagagcgagactccatctcaaaaaaaaaaaaaaaaaaaccacataggctcagtcttttcagtatctgctttactggttcagtaaaagccaggaaacacaactttgtggtaatctgaatgttattgaactgtattttgttcactttattgtaaatactagtgaacagtgaataaatggttgtatattcctaataagaaaaaaaaaaaaaaagacccaaagtacagcgagctgatgccgatctcatttcgcagaggtccgcctgctctcccctctccaagagtgtaatcctatgcttaataaacttatgccgctttgctatgtgtgtgtatcacacccaattctttgttcgaaacaccaagggcctggaacttcacagctttggctggtaacggggagcaggggtaaagacatttaaaagctgcttgtgttaaccataatcgccatcccatatatcagacccccagaactaactcatcttataactgaatattgtgctttttttttttttttttttttgagacgaagtcctgctctgtcacccaggctggagtgcagtggcgcgatcttgactctgcaacctccgcctcccgggttcaagcgattctcctgcctcagcctcccgagtagctgggactacaagtgcgtgccaccacgcccggctaatttttgtatttttagtagagacggggtttctccatgttggtctcaaactcctggtctcaggtgatccacccgccttggcctcccaaagtgctgggattacagacgtgagccaccacacccagctacttgtgctttttgaccaacatcttcctctcctaccacccccagcccctgataacctccacctactctcacttctaggagatcaactgttctatttttttttttttttttttttttttgagtctcgctctgcacacccaggctggagtgcagtgctgcaatctcggatcactgcaacctccgctttccgggttcaagcgattctcctgcctcagcctccagagtcgctgggattactgagccaccgcgcccagccagaagacccacgctccctaagacataacccacactggtggcctttgttctgacttctcacctgtgctccccacccgctagaaactggcttctctccccacacttcctctgaagctgtctgtgtgaccaacactaatgagcttccttcctggaacatgcagtgacccttttcagcccttctcattattgctcccccacagttgtatttgacacgttgaccacttcctcctcgaaggactcacttctctggctttctcggacacttcttgctactcgttttctgacggttacagtaccaacaggtttgcaggcacctccaccaccagagccaatcccagctactcgggaggctgaggcaggagaatcgttcaaacccgggaggcagaggttgcagtgagtcgagattgcgccactgcactccagcctgagtgacagactgtgactcctcaaaaaaaaacaaaaacaaaaacaaaaaaactacagtcttgctctgtcgcccaggatggaatgcagtggtgccatcttggctcactgcaacctctgcctgctggggtctagcgattctcctgcctcagccccccaaggagctgggactacaggcatgtgccgccacgcctggctaatttttgtatttttagtggagatgggggttttaccatgttagccaggttggtcttgaactcccgacctcatgtgatccgcccaccttggcctcccaaagtgcgaggattacaggcccccgcacccagcctaggatcctgcacctctctagcctagcagttctctgctgggtgattttgctctccactccaggggacatttggcaatgcccatggtaatttttaattgtcatgacttggggaggggttctactggcatctggtaggtagggtccaggggtgctgctcagcttcctacaatgcccagggcagccccagatggcagcagcaccaaggctgagaaacactggctcatgcagaaagcaaccaccttacacccttcagtgcagggacaaaggcagggttacgagtccacggaaactctccagtctcagcctacgtaagacgtggctatttttctttcttattgtttttattcatttatttttcttgagacagagtcttgctctgtcgcccaggctggactgcagtggcgcgatctctgctcactgcaagctccgcctcccgggatcacaccattctcctgggactacaggcgcccgccacctagcccggctaattttttgtatttttagtagagacggggtttcaccatgttagccaggatggtctcgatctgacctcgtgatcctcccgcctcggcctctcaaagtgctgggattacaggtgtaagccaccgcacccggccttattcatttattttttgagatagagtctgagccctttattttatttatttagagaccaagtctcgctctgttacccaggctggagtgcagtgtcgtggcctcagctcactgcaacaacctccgcctcccgggttcaagcgattctcccaccttggcctcccaaagtgctggcattacagacacccactaccatgcctggctaattttttgtacttttagtaagtaaagacagggtttcaccatcttggtcaggatggtctcgaactcctggcctcaagtgatcggcccgcctgggtctcccaaagtgatgagattacaggcgtgagcgaccacactggcctaatgtgtagttttttatctgtggcctcccttctgccctcccccttctgagactctgaagcccattacatcactctgcctttgtgtaccaacagcttagctcccactgagaacatacagagccaggcacggtggcggtggctcacgcctgtaatcccatcactttgggggtgctgaggcaggtgtatcgcctgaggccaggagttcaagaccagtctggccaacatggtgaaaccccatctctactaaaaatagaaaaatacatagctgggtgtggtggcacgtgcctataatcccagctactagggaggctgaggttggagaatcgcttgaacccaggaggcggaggttgcggtgagccaagatcacaccattgcactctagcctgggcaacaagagcaaaactgtcttaaaaaaaaaaaaaaaaagtgagaacatatggattctactcctgttagaataatggcctccagctccatccaaattgctggaaatgacattatttcattccttctaatggctgaatagtattccatggtacatagacaccacgttttctttatccactgtagggaccagccccacagggtcggtgggtctctccctgtgtgcggcgacgagagagtgtagaaataaagacacaagacaaagagacaagagaaaaggcagctgggcccgggggaccactaccaccaatgcgcggagaccggtagtggccccgaatgtctggctgcgctgttatttattggatacaaggcagaaggggcagggtaaagagtgtgagtcacctccaatgataggtaaggtcacgtgggtcacgtgtccactggacagggggcccttccctgcctggcagccgaggcagagagggagaggagacagagagaaagacagcttacgccattatttctgcatatcagggactattagtactttccctaatttactactgctatctagaaggcagagccaggtgtacaggatggaacatgaaggcggactaggagcgtgaccaccgaagcacagcatcacagggagacggttaggcctccggataactgcgggcgagcctgactgatgtcaggccctccacaagaggtggaggagcagagtcttctctaaactcccccggggaaagggagacccccccccccacccgctgcccctttcccggtctgctaagtagcgggtgttgttaattgacaccttttgctaccgctggaccatgatccgcttggtgacgggtgtcttcccagacgctggcgtcaccgctagaccaaggagccctctggtggccctgtccgggcataacagaaggctcgcactcttgtcttctggtcacacctcactatgtcccctcagctcctatctctgtatggcctggtttttcctaggctatgattatagagtgaggattattataatattggaataaaaagtaattgctaccggctaatgattaatgatactcatatataatcatatctaagatctatatctggtataacaattcttgttttatattttattatactggaacagctcgtgtcctctgtctcttgcctcggtgcctgggtgccttgccgcccacaatccactcattattcaatgggcacttcggttggttccacatctttgcaattgtgaatggctgagccagccattcttaactgggggtgattttgtccccatgggggtatctggccacatcccgagaggttttttggttgtcacgagttgcagtgggggcaggctcaggctcatccaagtccaggggtgctgctatacatcacgtgatacacaggacagtccttgctacggactgaattggtcccaccaaacgtcatgtacaagccctaccccagatgtgactctatttggacacagggcttttcagaggtaattaaggctggtcaggcgccgtaatcacagcactttaggagttctgtgtttattactggtaagtgggtaagagcccagtgtggcagctcacgcgtgtaatcccagcactttgggaagcgaaggcaaggggataactggaggccagcagttcaagacaagcctggtcaatacagcaagactccatctctataaaatattttaaaattagccaagcatgtttggcatgcacctgtaatcccagctcaggaggctcaggtgggaggattccttgagtttaaggctgcagtgagctaagatcgcaccattgcactccaacccggctgtgggcaacacagcaccaccaccatcttggctgggcacggtggctcacgcctgtcatgccagcactttgggaggccgaggcgggtggctcacctgaggtcaggagtttgagactagcctggccaacatggtgaaatcacgccactgcactccagcctaggcaaccaagtgagactctgtccgccccaccaccccaccaaaaaaaagactactatcttaaacaaaatcaaaatttttaagtagataaaatatttaggggaaaaaaacttcaattaaatatgcagcagagtccgacccagatgttttcactcccagcctctacctactatctttgtgtctttatttttagcaaattctacacgggaacttcatgtgcatgtagaaccctaaatgttgactcagccctacctctcatcacctgaccacttcctttattcacgctgtctctaccacccttcccatcggtgtgagctgtatcccgctaaacactgttaccacccacagcctgcattactaccagctgactgtagccttaaacaccacagtgatctcgagcatttgagaagacttatcttgacaagggctcacgaaagacagcaatgctcaacagcaagataaatgagggccttcatgggatcattcagtgctgaagccactcaacctccaggtttgggttagtaaaaagaactttgt